SEQUENCE LISTING (38 sequences)

SEQ ID NO: 1 - protein, 50 aa, Synthetic construct
DLISNINVIVLELKGSETTFMCEYADETATIVEFLNRWITFCQSIISTLT

SEQ ID NO: 2 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSP

SEQ ID NO: 3 - protein, 225 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 4 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 5 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 20 aa, Synthetic construct
GGGGSGGGGSGGGGSGGGGS

SEQ ID NO: 7 - protein, 551 aa, Synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 8 - protein, 551 aa, Synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 9 - protein, 551 aa, Synthetic construct
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 10 - protein, 153 aa, Homo sapiens [sequence omitted]

SEQ ID NO: 11 - protein, 575 aa, Synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 12 - protein, 542 aa, Synthetic construct
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 13 - protein, 542 aa, Synthetic construct; identical to SEQ ID NO: 12

SEQ ID NO: 14 - protein, 144 aa, Homo sp.
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 15 - protein, 560 aa, Synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 16 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 560 aa, Synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 18 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 19 - protein, 306 aa, Synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 20 - protein, 547 aa, Synthetic construct
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 21 - protein, 129 aa, Synthetic construct
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 22 - protein, 788 aa, Synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 23 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 24 - protein, 558 aa, Synthetic construct
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVVGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 25 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 26 - protein, 302 aa, Synthetic construct
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 27 - protein, 133 aa, Synthetic construct
APTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 153 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 29 - protein, 552 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - DNA, 1659 bp, Synthetic construct
  source 1..1659: mol_type = other DNA
atgtatagaatgcaactgctctcctgcatagcattaagcctcgccttagtcactaatagcgctccgacatcaagtagtacgaagaaaacacaactgcaacttgagaatcttctactggatctccagatgatccttaacggtatcaacaactataagaatcccaagttaacacaaatgttgacctttaaattctacatgcccaagaaggccacagaattgaagcatctccaatgtcttgaggaagagctgaaaccccttgaggaagtgttaaatctggcacagtctaagaactttcatctccgccccagagatttaatatctaatatcaatgtgatcgttctggaactgaagggttcagaaaccactttcatgtgcgagtatgctgatgagacagccacaattgtagaattcctcaacaggtggataacattctgccagtccatcattagtacattaactggtggcggtggatcagggggaggcggatcaggtggcggaggatccggtggaggaggttccgataagacccatacttgtccaccgtgtccagctccagaattgttgggtggacctagcgtgtttctgttcccacccaagcccaaagatacacttatgatcagtcggactcctgaggtaacgtgcgtggtagtggacgtcagccatgaggacccagaggtcaaatttaactggtatgttgatggagttgaagtgcataacgccaaaactaaaccaagggaagaacagtacaactctacctatagagtggtttccgtactcacagtactgcaccaagactggctgaacggaaaagaatataaatgtaaggtgagcaataaagcattgcccgccccgatcgagaaaacaatttcaaaagctaaaggccaaccgagagaaccccaagtttatacccttccaccctctcgggatgagttgaccaagaaccaagtgtcattaacttgtctggtgaagggattttatccctctgacattgccgtggaatgggaatccaacggtcagccggaaaacaattataaaactactccacccgtcttagactctgacgggagtttcttcctgtacagcaagcttacagtagacaagtctcggtggcaacaaggaaacgtcttcagctgtagcgtcatgcatgaagcattacataatcactatacacaaaagtctctgtcactgtcccctggcggtggaggaggcagcggcggtggtggttctggaggaggtggaagtggtggcggcgggagtgcaccaactagcagttccaccaaaaagactcagttacagctcgaaaacctgctgcttgacctgcagatgattctgaatggaataaataattacaaaaaccctaagctcacacagatgcttacgttcaagttttatatgccaaagaaggctacggagctcaaacacctgcaatgcttggaagaggaactcaagccattggaagaggttctgaacctggctcaatccaaaaatttccaccttaggcctagagacctcatcagcaacataaacgttatagtactagagctcaaagggagcgagacgacctttatgtgcgagtacgccgatgaaactgctaccatcgtggagtttttgaatagatggatcaccttttgtcaaagcattatatccacacttacctga

SEQ ID NO: 31 - protein, 398 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - DNA, 1197 bp, Synthetic construct
  source 1..1197: mol_type = other DNA
atgtatcgcatgcaactcttgtcttgtattgccctctctctcgccctcgtcacgaactctgctccaacaagtagttcaaccaagaaaacccaattacaattggaaaacttgctgctggacttacagatgatacttaacggcatcaataactacaagaacccaaagctcacacaaatgcttacatttaaattctatatgccgaagaaagcaacagagctcaagcatctccaatgcctcgaagaagagttaaagcctctcgaggaagttctcaatcttgcccaatccaagaactttcatcttagaccgcgagatctgatatctaatataaatgtgattgtccttgaactcaaagggtcagagactactttcatgtgtgagtatgccgatgagacggctaccatcgtggagttcctcaataggtggatcacgttttgccaaagcataatttccaccttgaccggcggcggtggatccggtggaggagggagtggtggcggcggaagcggcggtggtggctccgataaaacacacacatgtcccccttgtcctgcaccagaactcctgggcggcccttcagtgttcctgtttcccccaaaacccaaagacacattgatgatctcccgcactcctgaggtgacgtgtgttgtcgtggatgtttcccatgaagacccggaagttaaatttaactggtacgtggatggcgtagaagtccataatgccaagacaaagccacgggaagagcagtacaactctacctatcgagtggtctcagtgttgaccgttctgcaccaagattggctgaatgggaaggagtataagtgtaaggtttcaaacaaagctctgccggcacccattgaaaagacaatctctaaggcaaaaggccaaccaagagagccacaagtctatacgctgccccctagccgggatgagctcactaagaaccaggtatctctcacatgtctcgtcaaagggttctatccatctgatatcgcagtggaatgggaatcaaatggacagccagagaacaattataaaaccactccacccgtccttgattccgacgggtccttcttcttatattccaagttaactgtggataaatctcgttggcagcaaggcaatgtctttagttgctccgtaatgcatgaagcgctccataaccattacacacagaagtcactgtcactcagcccttaa

SEQ ID NO: 33 - protein, 398 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSP

SEQ ID NO: 34 - DNA, 1197 bp, Synthetic construct
  source 1..1197: mol_type = other DNA
atgtaccgaatgcaattgcttagctgtattgccttgagtctcgcactcgtgactaactcagcccccacctcatccagcactaagaaaacgcagctgcaactcgagaacttactgcttgatctccagatgattctcaacggcatcaacaattacaagaatccaaagttgacacaaatgctgacctttaaattttatatgccaaagaaggccaccgagctgaagcatttgcagtgccttgaagaggaacttaaaccactggaagaagttttgaacctggcccaaagcaagaacttccacttgagacctcgggatctgataagcaacattaacgttattgtcctggagctcaaaggatcagaaacaaccttcatgtgtgaatacgcagatgagactgcaactattgtagagtttctgaaccgctggattaccttctgccagagcattatttccacactcacaggcggaggaggatcaggcggcggcggtagcggcggaggaggaagcggaggaggcggcagtgataagacacatacatgtccaccttgtccagccccagagcttttgggcggcccgtccgtgtttctgtttccacctaagcctaaagatacattaatgatcagccggactcctgaagttacttgtgttgtggttgatgtcagtcatgaggatccagaggttaagttcaactggtacgtggatggtgtggaagttcacaatgcgaaaacgaaaccacgcgaagaacagtataatagtacataccgtgtagtgagcgtattgactgtgctccatcaagactggcttaatggcaaagaatataagtgtaaggtgagtaacaaagcgctccccgcacctattgagaagacgattagcaaagccaagggtcaaccacgcgaaccccaagtctatacactgcccccttcccgtgatgagttaaccaagaatcaagtcaatcttacctgtcttgtaaaaggtttctacccgtctgatatcgccgtcgaatgggagtctaatggccaaccggaaaacaactacaagaccacaccgcccgtcctcgactcagatgggagtttcttcctgaatagcactttgacagtggataaaagtcgatggcaacaggggaatgtgttctcctgtagcgtaatgcatgaggctctgcataaccattacacacagaaatcactcagtctcagcccttaa

SEQ ID NO: 35 - protein, 551 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 36 - DNA, 1656 bp, Synthetic construct
  source 1..1656: mol_type = other DNA
atgtatagaatgcaactgctctcctgcatagcattaagcctcgccttagtcactaatagcgctccgacatcaagtagtacgaagaaaacacaactgcaacttgagaatcttctactggatctccagatgatccttaacggtatcaacaactataagaatcccaagttaacacaaatgttgacctttaaattctacatgcccaagaaggccacagaattgaagcatctccaatgtcttgaggaagagctgaaaccccttgaggaagtgttaaatctggcacagtctaagaactttcatctccgccccagagatttaatatctaatatcaatgtgatcgttctggaactgaagggttcagaaaccactttcatgtgcgagtatgctgatgagacagccacaattgtagaattcctcaacaggtggataacattctgccagtccatcattagtacattaactggtggcggtggatcagggggaggcggatcaggtggcggaggatccggtggaggaggttccgataagacccatacttgtccaccgtgtccagctccagaattgttgggtggacctagcgtgtttctgttcccacccaagcccaaagatacacttatgatcagtcggactcctgaggtaacgtgcgtggtagtggacgtcagccatgaggacccagaggtcaaatttaactggtatgttgatggagttgaagtgcataacgccaaaactaaaccaagggaagaacagtacaactctacctatagagtggtttccgtactcacagtactgcaccaagactggctgaacggaaaagaatataaatgtaaggtgagcaataaagcattgcccgccccgatcgagaaaacaatttcaaaagctaaaggccaaccgagagaaccccaagtttatacccttccaccctctcgggatgagttgaccaagaaccaagtgtcattaacttgtctggtgaagggattttatccctctgacattgccgtggaatgggaatccaacggtcagccggaaaacaattataaaactactccacccgtcttagactctgacgggagtttcttcctgtacagcaagcttacagtagacaagtctcggtggcaacaaggaaacgtcttcagctgtagcgtcatgcatgaagcattacataatcactatacacaaaagtctctgtcactgtcccctggtggaggaggcagcggcggtggtggttctggaggaggtggaagtggtggcggcgggagtgcaccaactagcagttccaccaaaaagactcagttacagctcgaacatctgctgcttgcactgcagatgattctgaatggaataaataattacaaaaaccctaagctcacacgtatgcttacgttcaagttttatatgccaaagaaggctacggagctcaaacacctgcaatgcttggaagaggaactcaagccattggaagaggttctgaacctggctcaatccaaaaatttccaccttaggcctagagacctcatcagcaacataaacaagatagtactagagctcaaagggagcgagacgacctttgtttgcgagtacgccgatgaaactgctaccatcgtggagtttttgaatagatggatcaccttttgtcaaagcattatatccacacttacctga

SEQ ID NO: 37 - protein, 551 aa, Synthetic construct
MYRMQLLSCIALSLALVTNSAPTSSSTKKTQLQLENLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGGGGSGGGGSGGGGSGGGGSAPTSSSTKKTQLQLEHLLLALQMILNGINNYKNPKLTRMLTFKFYMPKKATELKHLQCLEEELKPLEEVLNLAQSKNFHLRPRDLISNINKIVLELKGSETTFVCEYADETATIVEFLNRWITFCQSIISTLT

SEQ ID NO: 38 - DNA, 1656 bp, Synthetic construct
  source 1..1656: mol_type = other DNA
atgtatagaatgcaactgctctcctgcatagcattaagcctcgccttagtcactaatagcgctccgacatcaagtagtacgaagaaaacacaactgcaacttgagaatcttctactggatctccagatgatccttaacggtatcaacaactataagaatcccaagttaacacaaatgttgacctttaaattctacatgcccaagaaggccacagaattgaagcatctccaatgtcttgaggaagagctgaaaccccttgaggaagtgttaaatctggcacagtctaagaactttcatctccgccccagagatttaatatctaatatcaatgtgatcgttctggaactgaagggttcagaaaccactttcatgtgcgagtatgctgatgagacagccacaattgtagaattcctcaacaggtggataacattctgccagtccatcattagtacattaactggtggcggtggatcagggggaggcggatcaggtggcggaggatccggtggaggaggttccgataagacccatacttgtccaccgtgtccagctccagaattgttgggtggacctagcgtgtttctgttcccacccaagcccaaagatacacttatgatcagtcggactcctgaggtaacgtgcgtggtagtggacgtcagccatgaggacccagaggtcaaatttaactggtatgttgatggagttgaagtgcataacgccaaaactaaaccaagggaagaacagtacaactctacctatagagtggtttccgtactcacagtactgcaccaagactggctgaacggaaaagaatataaatgtaaggtgagcaataaagcattgcccgccccgatcgagaaaacaatttcaaaagctaaaggccaaccgagagaaccccaagtttatacccttccaccctctcgggatgagttgaccaagaaccaagtgaatttaacttgtctggtgaagggattttatccctctgacattgccgtggaatgggaatccaacggtcagccggaaaacaattataaaactactccacccgtcttagactctgacgggagtttcttcctgaatagcacccttacagtagacaagtctcggtggcaacaaggaaacgtcttcagctgtagcgtcatgcatgaagcattacataatcactatacacaaaagtctctgtcactgtcccctggtggaggaggcagcggcggtggtggttctggaggaggtggaagtggtggcggcgggagtgcaccaactagcagttccaccaaaaagactcagttacagctcgaacatctgctgcttgcactgcagatgattctgaatggaataaataattacaaaaaccctaagctcacacgtatgcttacgttcaagttttatatgccaaagaaggctacggagctcaaacacctgcaatgcttggaagaggaactcaagccattggaagaggttctgaacctggctcaatccaaaaatttccaccttaggcctagagacctcatcagcaacataaacaagatagtactagagctcaaagggagcgagacgacctttgtttgcgagtacgccgatgaaactgctaccatcgtggagtttttgaatagatggatcaccttttgtcaaagcattatatccacacttacctga

(19) United States
(12) Patent Application Publication
(10) Pub. No.: US 2026/0201007 A1
(43) Pub. Date: Jul. 16, 2026

(54) MULTISPECIFIC ENGINEERED BIOMOLECULES AND USES THEREOF

(71) Applicant: The Regents of the University of California, Oakland, CA (US)

(72) Inventors: Aaron S. MEYER, Los Angeles, CA (US); Brian ORCUTT-JAHNS, Los Angeles, CA (US); Peter EMMEL, Los Angeles, CA (US)

(73) Assignee: The Regents of the University of California, Oakland, CA (US)

(21) Appl. No.: 19/133,902

(22) PCT Filed: Nov. 30, 2023
(86) PCT No.: PCT/US23/81752

§ 371 Date: May 29, 2025

Related U.S. Application Data
(60) Provisional application No. 63/428,921, filed on Nov. 30, 2022.

(51) Int. Cl. C07K 14/55; A61K 31/7105; A61K 38/00; A61P 35/00; C07K 14/525; C07K 14/54; C07K 14/555; C12N 15/11; C12N 15/63

(52) U.S. Cl. CPC C07K 14/55; A61K 31/7105; A61K 38/00; A61P 35/00; C07K 14/525; C07K 14/5406; C07K 14/5418; C07K 14/5425; C07K 14/5443; C07K 14/555; C12N 15/11; C12N 15/63; C07K 2317/21; C07K 2317/31; C07K 2317/35; C07K 2317/52; C07K 2319/02; C07K 2319/30

(57) ABSTRACT

The present disclosure provides a multispecific multivalent biomolecule comprising two or more covalently linked cytokines or variants thereof, wherein at least one cytokine is signaling-competent such as the native cytokine or a signaling-competent variant thereof, and at least one of the same or different cytokine is signaling-deficient compared to its native cytokine. The multispecific multivalent biomolecule presented herein can be used to activate or suppress immune responses in a subject.

### CROSS-REFERENCE TO RELATED APPLICATIONS

This application claims priority to U.S. Provisional patent application Ser. No. 63/428,921, filed Nov. 30, 2022, which is incorporated herein by reference in its entirety.

### GOVERNMENT SUPPORT

This invention was made with government support under AI148119 awarded by the National Institutes of Health. The government has certain rights in the invention.

### FIELD

The present disclosure is related in general to the field of cytokine signaling. In one embodiment, the present disclosure provides strategies for altering cytokine signaling through changes in binding affinity in combination with valency using heterospecific fusions.

### BACKGROUND

Cytokines are small proteins involved in cell signaling, including autocrine, paracrine, endocrine signaling as well as immunomodulating agents. They include chemokines, interferons, interleukins, lymphokines, and tumor necrosis factors. Cytokines are produced by a broad range of cells, including immune cells such as macrophages, B lymphocytes, T lymphocytes and mast cells, as well as endothelial cells, fibroblasts, and various stromal cells; a given cytokine may be produced by more than one type of cell. Cytokines act through cell surface receptors and are especially important in the immune system; they modulate the balance between humoral and cell-based immune responses, and they regulate the maturation, growth, and responsiveness of particular cell populations. Some cytokines enhance or inhibit the action of other cytokines in complex ways. These proteins are important in health and disease, specifically in host immune responses to infection, inflammation, trauma, sepsis, cancer, and reproduction. Among cytokines, interleukins, which are expressed by and secreted by white blood cells and other body cells, modulate immune function, and are involved in the development and differentiation of T and B lymphocytes and hematopoietic cells. There are over 50 interleukins and related proteins encoded in the human genome.

Originally identified as the third subunit of the high-affinity IL-2 receptor complex, the common γ-chain (γ_{c}) also acts as a non-redundant receptor subunit for a series of other cytokines, collectively known as γ_{c} family cytokines. Cytokines belonging to the common cytokine receptor gamma chain family include IL-2, IL-4, IL-7, IL-9, IL-15, and IL-21. Members of this family signal through receptor complexes that contain the common gamma chain subunit. This subunit associates with different cytokine-specific receptor subunits to form unique heterodimeric receptors for IL-4, IL-7, IL-9, and IL-21, or associates with both IL-2/IL-2Rβ and IL-2Rα or IL-15Rα to form heterotrimeric receptors for IL-2 or IL-15, respectively. Common gamma chain family cytokines generally activate three major signaling pathways that promote cellular survival and proliferation: the PI3K-Akt pathway, the RAS-MAPK pathway, and the JAK-STAT pathway.

Consistent with the involvement of γ_{c} in diverse cytokine receptor complexes, the chain is expressed constitutively by multiple hematopoietic cell types, including macrophages and T, B and NK cells. Unlike most other cytokine receptors, γ_{c} is thought to be constitutively expressed and functions only after the assembly into receptor complexes.

Common gamma chain family cytokines serve as critical regulators of the development, survival, proliferation, differentiation and/or function of multiple immune cell types. These cytokines can have both unique and overlapping effects on different cell types, depending primarily on the expression patterns of the cytokines and their unique receptor subunits. Inactivating mutations in the common gamma chain family cytokines, their receptors, or a subset of intracellular signaling molecules involved in these pathways can lead to severe immune system defects. The most common form of severe combined immunodeficiency, X-linked SCID, is caused by mutations in the common cytokine receptor gamma chain subunit.

Many therapies, such as engineered antibodies, cytokines, and chimeric antigen receptors, aim to specifically expand, reduce, or alter the function of specific cells within the body. These strategies derive their specificity from the unique surface profiles of a target population. Specificity must be designed to overcome challenges from cell heterogeneity, subtle distinctions from off-target populations, and uniqueness that is only found through combinations of markers. These challenges make quantitative strategies for engineering cell specificity critical.

Cytokines that bind to the common γ-chain (γ_{c}) receptor, such as interleukin (IL)-2, 4, 7, 9, 15, and 21, are a critical hub in modulating both innate and adaptive immune responses. The cytokine family operates through a common theme of binding private receptors for each ligand before engaging the common γ_{c} receptor to induce signaling. A prominent phenotypic outcome of γ_{c} receptor signaling is lymphoproliferation, and so the cytokines are often observed to be an endogenous or exogenous mechanism for altering the balance of immune cell types. This phenotype is observed most extremely from loss-of-function or reduced activity mutations in γ_{c} which subvert T and NK cell maturation. Disruptive mutations in private receptors can lead to more selective reductions in cell types such as regulatory T cells (T_{reg}s) with IL-2Rα or T cells with IL-7Rα. Conversely, activating mutations in these receptors, such as IL-7Rα, promote cancers such as B and T cell leukemias.

The importance of these cytokines to immune homeostasis and challenges in altering their signaling toward specific therapeutic goals have inspired a variety of engineered forms. The most common approach has been to alter the receptor affinities of IL-2 to weaken its interaction with IL-2Rα, IL-2Rβ, or both receptors. IL-2Rα confers T_{reg}s with greater sensitivity toward IL-2, and so IL-2Rα affinity tunes the relative amount of signaling toward regulatory versus effector populations, while IL-2Rβ modulates the overall signaling potency. In most cases, the wild-type cytokine or mutein is fused to an IgG antibody to take advantage of FcRn-mediated recycling for extended half-life. Fc fusion has taken many forms, including orienting the cytokine in an N-terminal or C-terminal orientation, including one or two cytokines per IgG, and including or excluding Fc effector functions. The potential design space for these molecules quickly becomes experimentally intractable without consistent design principles.

Thus, there is a need to further evaluate the signaling effects of specific engineered cytokine alterations, e.g., affinity-altering mutations and Fc-fusion formats, in the design of strategies to alter signaling toward specific therapeutic goals.

### SUMMARY

In one aspect, the present disclosure provides a multispecific multivalent biomolecule comprising two or more covalently linked cytokines or variants thereof, wherein the biomolecule comprises at least a first cytokine or variant thereof, and at least a second cytokine or variant thereof, wherein:
a. the first cytokine or variant thereof is a signaling-competent cytokine or a signaling-competent variant thereof; and
b. the second cytokine or variant thereof is a same or different cytokine from the first cytokine, and is signaling-deficient compared to that of a native second cytokine.

In some embodiments, the signaling-deficient cytokine has increased receptor affinity, decreased receptor affinity, increased receptor signaling, decreased receptor signaling, or any combination thereof. In some embodiments, decreased receptor signaling is substantially no receptor signaling.

In some embodiments, the first cytokine or variant thereof and the second cytokine of variant thereof are the same cytokine. In some embodiments, the first cytokine or variant thereof and the second cytokine or variant thereof are different cytokines. In some embodiments, the multispecific multivalent biomolecule comprises at least 2, at least 3, at least 4, at least 5, at least 6, at least 7, or at least 8 cytokines or variants thereof. In some embodiments, the multivalent biomolecule comprises 2, 3, 4, 5, 6, 7 or 8 cytokines or variants thereof.

In some embodiments, the first cytokine or variant thereof and the second cytokine or variant thereof are covalently linked by being present on a fusion polypeptide. In some embodiments, the first cytokine or variant thereof and the second cytokine or variant thereof cytokines are covalently linked by cross-linking. In some embodiments, the cytokines are covalently linked by a first cytokine or variant thereof being present on a fusion polypeptide and cross-linked to another fusion polypeptide comprising the second cytokine or variant thereof. In some embodiments, the cytokines are covalently linked by cross-linking a fusion polypeptide comprising a first cytokine or variant thereof and the second cytokine or variant thereof to at least another cytokine.

In some embodiments, the cytokines or variants thereof are expressed as Fc fusion proteins of the cytokines or variants thereof with human IgG1 Fc. In some embodiments, the fusion protein comprises the cytokines or variants thereof fused to the N- or C-terminus of human IgG1 Fc. In some embodiments, the cytokines or variants thereof are fused to the N- or C-terminus of human IgG1 Fc through a (G₄S)₄ linker.

In some embodiments, at least the first cytokine or the second cytokine is a lymphokine, an interferon, an interleukin, a chemokine or tumor necrosis factor. In some embodiments, at least the first cytokine or the second cytokine is a common γ-chain receptor cytokine. In some embodiments, both the first cytokine and the second cytokine are a common γ-chain receptor cytokine. In some embodiments, the common γ-chain receptor cytokine is independently one or more of IL-2, IL-4, IL-7, IL-9, IL-15 or IL-21, or a variant thereof.

In one aspect, the present disclosure provides a multispecific multivalent biomolecule as described herein comprising two or more covalently linked cytokines or variants thereof, wherein the biomolecule comprises at least a first cytokine or variant thereof, and at least a second cytokine or variant thereof, wherein:
a. the first cytokine or variant thereof is a signaling-competent common γ-chain receptor cytokine or a signaling-competent variant thereof; and
b. the second cytokine or variant thereof is a same or different common γ-chain receptor cytokine from the first cytokine, and is signaling-deficient compared to that of a native second cytokine.

In some embodiments, at least one common γ-chain receptor cytokine, or variant thereof, comprises a signal sequence. In some embodiments, the signaling-deficient cytokine has at least one mutation. In some embodiments, the at least one mutation is an inactivating mutation.

In some embodiments, the multispecific multivalent biomolecule comprises a signaling-competent IL-2 and a signaling-deficient IL-2. In some embodiments, the signaling-competent IL-2 is native IL-2 or IL-2 having a R38Q and/or H16N mutation. In some embodiments, the signaling-deficient IL-2 has a V91K, D20A or M104V mutation, or any combination thereof. In some embodiments, the multispecific multivalent biomolecule comprises two signaling-competent IL-2 and two signaling-deficient IL-2 muteins.

In some embodiments, the multispecific multivalent biomolecule comprises a signaling-competent IL-4 and a signaling-deficient IL-2 mutein. In some embodiments, the signaling-competent IL-4 is native IL-4. In some embodiments, the signaling-deficient IL-2 has a V91K, D20A or M104V mutation, or any combination thereof. In some embodiments, the multispecific multivalent biomolecule comprises two signaling-competent IL-4 and two signaling-deficient IL-2 muteins.

In some embodiments, the multispecific multivalent biomolecule is SEQ ID NOs: 07, 08, 09, 11, 13, 15, 17, 19, 20, 22, 24, 26, 29, 31, 33, 35 or 37.

In some embodiments, the multispecific multivalent biomolecule comprises a signaling-competent IL-4 and a signaling-deficient IL-4. In some embodiments, the signaling-competent IL-4 is native IL-4 or IL-4 having R121D/Y124D mutation. In some embodiments, the signaling-deficient IL-4 has a R121D/Y124D mutation, or any combination thereof. In some embodiments, the multispecific multivalent biomolecule comprises two signaling-competent IL-4 and two signaling-deficient IL-4 muteins.

In some embodiments, the multispecific multivalent biomolecule comprises a signaling-competent IL-7 and a signaling-deficient IL-2 mutein. In some embodiments, the signaling-competent IL-7 is native IL-7. In some embodiments, the signaling-deficient IL-2 has a V91K, D20A or M104V mutation, or any combination thereof. In some embodiments, the multispecific multivalent biomolecule comprises two signaling-competent IL-7 and two signaling-deficient IL-2 muteins.

In some embodiments, the multispecific multivalent biomolecule comprises a signaling-competent IL-9 and a signaling-deficient IL-2 mutein. In some embodiments, the signaling-competent IL-9 is native IL-9. In some embodiments, the signaling-deficient IL-2 has a V91K, D20A or M104V mutation, or any combination thereof. In some embodiments, the multispecific multivalent biomolecule comprises two signaling-competent IL-9 and two signaling-deficient IL-2 muteins.

In some embodiments, the multispecific multivalent biomolecule comprises a signaling-competent IL-15 and a signaling-deficient IL-2 mutein. In some embodiments, the signaling-competent IL-15 is native IL-15. In some embodiments, the signaling-deficient IL-2 has a V91K, D20A or M104V mutation, or any combination thereof. In some embodiments, the multispecific multivalent biomolecule comprises two signaling-competent IL-15 and two signaling-deficient IL-2 muteins.

In some embodiments, the multispecific multivalent biomolecule comprises a signaling-competent IL-21 and a signaling-deficient IL-2 mutein. In some embodiments, the signaling-competent IL-21 is native IL-21. In some embodiments, the signaling-deficient IL-2 has a V91K, D20A or M104V mutation, or any combination thereof. In some embodiments, the multispecific multivalent biomolecule comprises two signaling-competent IL-21 and two signaling-deficient IL-2 muteins.

In some embodiments, the multispecific multivalent biomolecule comprises cross-linked SEQ ID NO:7, cross-linked SEQ ID NO:20, cross-linked SEQ ID NO:07 and SEQ ID NO: 20, cross-linked SEQ ID NO:22, cross-linked SEQ ID NO:24, cross-linked SEQ ID NO: 29, cross-linked SEQ ID NO:31, cross-linked SEQ ID NO:35, cross-linked SEQ ID NO:29 and SEQ ID NO:20, cross-linked SEQ ID NO:7 and SEQ ID NO:35, cross-linked SEQ ID NO: 31 and SEQ ID NO:7, cross-linked SEQ ID NO:31 and SEQ ID NO:20, cross-linked SEQ ID NO: 31 and SEQ ID NO:22, cross-linked SEQ ID NO:31 and SEQ ID NO:24, or cross-linked SEQ ID NO:31 and SEQ ID NO:29.

In some embodiments, the multispecific multivalent biomolecule consists of two cross-linked SEQ ID NO:7, two cross-linked SEQ ID NO:20, cross-linked SEQ ID NO:07 and SEQ ID NO: 20, two cross-linked SEQ ID NO:22, two cross-linked SEQ ID NO:24, two cross-linked SEQ ID NO:29, two cross-linked SEQ ID NO:31, two cross-linked SEQ ID NO:35, cross-linked SEQ ID NO:29 and SEQ ID NO:20, cross-linked SEQ ID NO:7 and SEQ ID NO:35, cross-linked SEQ ID NO:31 and SEQ ID NO:7, cross-linked SEQ ID NO:31 and SEQ ID NO: 20, cross-linked SEQ ID NO:31 and SEQ ID NO:22, cross-linked SEQ ID NO:31 and SEQ ID NO: 24, or cross-linked SEQ ID NO:31 and SEQ ID NO:29.

In some embodiments, the multispecific multivalent biomolecule has enhanced selectivity for driving T_{reg}-mediated immune suppression compared to a native cytokine.

In one aspect, a method is provided for modulating the immune system of a subject, comprising administering to a subject in need thereof any of the multispecific multivalent biomolecules disclosed herein or a nucleic acid encoding the multispecific multivalent biomolecules or cross-linkable components thereof. In some embodiments, the multispecific multivalent biomolecule is used for treating cancer. In some embodiments, the modulating is suppressing immune responses in the subject. In some embodiments, the multispecific multivalent biomolecule is used for treating an autoimmune disease or preventing transplant rejection. In some embodiments, the autoimmune disease is systemic lupus erythematosus.

In some embodiments of the method, the subject is administered a multispecific multivalent biomolecule or a component thereof, or the subject is administered a nucleic acid encoding a multispecific multivalent cytokine or component thereof. In some embodiments, the subject is administered cells exposed ex vivo or in vitro to a nucleic acid encoding a multispecific multivalent cytokine or component thereof.

In one aspect, a multispecific multivalent cytokine is provided of any one of SEQ ID NOs: 07, 08, 09, 11, 13, 15, 17, 19, 20, 22, 24, 26, 29, 31, 33, 35 or 37. In some embodiments, a multispecific multivalent cytokine is provided consisting of any one of SEQ ID NOs: 07, 08, 09, 11, 13, 15, 17, 19, 20, 22, 24, 26, 29, 31, 33, 35 or 37. In some embodiments, a multispecific multivalent cytokine comprises a cross-linked dimer of any one of SEQ ID NOs: 07, 20, 22, 24, 29, 31 or 35, or any homodimeric or heterodimeric combination thereof. In some embodiments, a multispecific multivalent cytokine consists of a cross-linked dimer of two of any of SEQ ID NOs: 07, 20, 22, 24, 29, 31 or 35, or any homodimeric or heterodimeric combination thereof.

In one aspect, a pharmaceutical composition is provided comprising any multispecific multivalent biomolecule or nucleic acid encoding any multispecific multivalent biomolecule or cross-linkable component thereof disclosed herein. In some embodiments, the pharmaceutical composition comprises SEQ ID NOs: 07, 08, 09, 11, 13, 15, 17, 19, 20, 22, 24, 26, 29, 31, 33, 35 or 37 or a homomeric heteromeric cross-linked dimer of any one of SEQ ID NOs: 07, 20, 22, 24, 29, 31, 35 or any combination thereof. In some embodiments, the pharmaceutical composition consists of SEQ ID NOs: 07, 08, 09, 11, 13, 15, 17, 19, 20, 22, 24, 26, 29, 31, 33, 35 or 37 or consists of a homomeric or heteromeric cross-linked dimer consisting of two of any of SEQ ID NOs: 07, 20, 22, 24, 29, 31, 35, or any combination thereof. In some embodiments, the pharmaceutical composition comprises a controlled release delivery composition or device.

In one aspect, a nucleic acid is provided encoding a multispecific multivalent cytokine as disclosed herein. In some embodiments, the nucleic acid encodes any one of SEQ ID NOs: 07, 08, 09, 11, 13, 15, 17, 19, 20, 22, 24, 26, 29, 31, 33, 35 or 37. In one embodiment, the nucleic acid is mRNA.

In one aspect, a vector or plasmid is provided comprising a nucleic acid encoding a multispecific multivalent cytokine as described herein. In some embodiments, the vector comprising a nucleic acid encoding any one of SEQ ID NOs: 07, 08, 09, 11, 13, 15, 17, 19, 20, 22, 24, 26, 29, 31, 33, 35 or 37.

In some embodiments, a pharmaceutical composition is provided comprising a nucleic acid, vector, plasmid or mRNA that encodes any of the cytokines disclosed herein. In some embodiments, the pharmaceutical composition comprises a nanoparticle such as a lipid nanoparticle.

These and other aspects of the disclosure will be appreciated from the ensuing descriptions of the figures and detailed description of the disclosure.

### BRIEF DESCRIPTION OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

FIGS. 1A-1O show that systematically profiling IL-2 muteins reveals determinants of response. (A) Schematic of affinity and structural mutants explored. (B) IL2Rα and IL2Rβ affinities of each IL-2 variant. For affinity assays, two technical replicates were conducted (N=2). (C) Heatmap of phosphorylated STAT5 measurements for each cell type, time point, ligand, and concentration. pSTAT5 measurements are normalized to the maximum pSTAT5 observed in response to WT IL-2 for each cell type. (D to O) STAT5 phosphorylation response curves for immune cells stimulated with select IL-2 muteins. Time points and cell types are indicated in subplot titles. For all signaling assays, PBMCs were collected from one donor, and three technical replicates were conducted (N=3).

FIGS. 2A-2N show that IL-2 muteins display structural- and affinity-dependent T_{reg} selectivity that cannot be overcome with cis-targeting strategies. (A) Schematic describing ratio of activation between target and off-target immune populations. (B and C) Ratio of T_{reg}-to-CD8+ T cell pSTAT5 dose-response curve at 4 hours (B), and the maximum ratio of signaling (pSTAT5) in T_{reg} cells to off-target cell type versus IL2Rα affinity (C). (D and E) As described in (B and C), respectively, of T_{reg} cells to NK cells (D and E). (F and G) NK^{bright} cells (F and G), and Tₕₑₗₚₑᵣ cells (H and I). The ratio was defined as the ratio of Hill curves fit to experimental data for target and off-target populations shown in FIG. 1. Lines of best fit were separately fit to monovalent (thin) and bivalent (thick). Signaling data in (B to I) was gathered from PBMCs harvested from a single donor, and three technical replicates were conducted (N=3). (J) Schematic depicting how useful markers for conferring selectivity are selected. (K to N) Top sorted Wasserstein distances (k, m) and Kullback-Leibler divergences (l,n) of surface markers (k,l) and RNA data (m,n) in T_{reg}s in the CITE-seq dataset (GSE164378).

FIGS. 3A-3H show that tensor-based decomposition reveals unique selectivity defined by fusion valency. (A) Principal components analysis scores (left) and loadings (right) of pSTAT5 signaling data. Signaling data was gathered from PBMCs harvested from a single donor, and three technical replicates were conducted (N=3). Principal component decomposition was performed on signaling data arranged in matrix form, where each dose and ligand combination is included as a row, and each cell and time combination is included as a column. (B) Schematic representation of non-negative canonical polyadic (CP) decomposition. Experimental pSTAT5 measurements are arranged in a tensor according to the duration of treatment, ligand used, cytokine concentration, and cell type. CP decomposition then helps to identify and visualize patterns across these dimensions. (C) Percent variance reconstructed (R2X) of the signaling dataset versus the number of components used during CP decomposition. (D) Component weights for each IL-2 mutant resulting from CP decomposition of the signaling dataset. (E) Component weights representing the effect of IL-2 concentration resulting from CP decomposition of the signaling dataset. (F) Component weights representing cell type specificity resulting from CP decomposition of the signaling dataset. (G) Component weights for the effect of treatment duration resulting from CP decomposition of the signaling dataset. (H) Sum of component 1 and 3 weights (off-target signaling) versus component 2 (T_{reg} signaling) weight for each monovalent and bivalent ligand. Thin line is included for visualization purposes only.

FIGS. 4A-4I show that responses are predicted by a simple multivalent binding model. (A) Schematic of the model. Initial association of multivalent ligands proceeds according to monovalent affinity, and subsequent binding events proceed with that affinity scaled by the Kt parameter. Model was fit to the signaling data gathered from PBMCs harvested from a single donor, and three technical replicates were conducted (N=3). Receptor counts used in model simulations for each cell population were measured in PBMCs gathered from a single donor, and four technical replicates were performed (N=4). (B and C) Model's accuracy subset by cell type (B) and ligand (C) for all monovalent and bivalent IL-2 muteins. (D and E) Model's accuracy subset by concentration (D) for all ligands and time (E) for all ligands, concentrations, and cell types. All accuracies (B to E) are calculated as a Pearson's correlation R2 score for experimental cytokine responses at 30 mins and 1 hour. (F and G) Model-predicted pSTAT for T_{reg}s (F) and NK cells (G) in response to mono- and bivalent IL-2 ligands with 10 nM IL2Rβ K_{D}. (H and I) Predicted number of active signaling complexes (proportional to predicted pSTAT5) formed on cells with 1000 IL2Rβ receptors and varying numbers of IL2Rα for ligands with affinities of 10 nM K_{D} for IL2Rβ and either 1 nM (f) or 10 nM (i) K_{D} for IL2Rα.

FIGS. 5A-5K show that multivalency enhances the selectivity of cytokine fusion proteins. (A and B) Predicted signaling response of T_{reg} cells in response to a ligand of optimal selectivity at different valencies (A), and optimal receptor-ligand dissociation constants for ligand optimized for selectivity (B). Response predictions were normalized to each population's response for the monovalent case. (C and D) As described in (A and B), respectively, of NK cells (C and D). (E and F) As described in (A and B), respectively, of Tₕₑₗₚₑᵣ cells (E and F). Selectivity for T_{reg} and NK cells were derived from IL-2 muteins, and selectivity for Tₕₑₗₚₑᵣs was calculated using IL-7 muteins. During affinity optimization (B, D, and F), mutein affinity for IL2Rα and IL2Rβ/γ_{c} was allowed to vary for IL-2 muteins, and affinity for IL7Rα was allowed to vary for IL-7 muteins. Affinities were allowed to vary across K_{D}s of 10 μM-1 μM while K*ₓ was fixed at its fitting optimum. All optimizations were performed using a concentration of 1 nM. Selectivity was calculated as the ratio of predicted pSTAT5 in target cells to the mean pSTAT5 predicted in off-target cells. Receptor counts used in model simulations for each cell population were measured in PBMCs gathered from a single donor, and four technical replicates were performed (N=4). (G) Schematic of multivalent IL-2 mutant design. (H to K) Ratio of STAT5 phosphorylation in T_{reg}s to Tₕₑₗₚₑᵣs (H), NK cells (I), NK^{bright} (J), and CD8⁺ (K) cells at varying dosages for R38Q/H16N in various valency formats. Dots are representative of mean of biological replicates. Responses in PBMCs from 5 donors were included, and two experimental replicates were conducted for each donor (N=5). Statistical significance was determined by comparing the ratios achieved by tetravalent R38Q/H16N to bivalent R38Q/H16N using a Student's t-test. Full ratio plots including experimental error are shown in figure S7.

FIGS. 6A-6L show that asymmetric IL-2 mutants display even greater T_{reg} selectivity. (A and B) Predicted enhancements to T_{reg} selectivity for IL-2 muteins including a separate targeting domain as calculated using CITE-seq surface marker data (A) or surface expressed RNA transcripts (B). Selectivity was calculated for T_{reg}s against all other PBMC cells as the increase in average T_{reg} to off-target cell binding against WT IL-2 at a simulated concentration of 0.1 nM. Model predicted signaling was predicted on a single-cell basis for cells within the CITE-seq dataset. (C) Schematic of asymmetric IL-2 mutant design. (D) Predicted normalized T_{reg} selectivity displayed by bivalent WT IL-2, IL2Rα biased IL-2 (IL2Rα affinity of R38Q/H16N), and Bitargeted IL-2 across IL2Rβ affinities. (E to G) Ratio of STAT5 phosphorylation in T_{reg}s to Tₕₑₗₚₑᵣ (E), NK (F), NK^{bright} (G) and CD8+ (H) cells at varying concentrations for R38Q/H16N in various valency formats. Dots are representative of mean of experimental replicates. Responses in PBMCs from 5 donors were included, and two experimental replicates were conducted for each donor. Statistical significance was determined by comparing the ratios achieved by bivalent bitargeted to bivalent R38Q/H16N using a Student's t-test. Full ratio plots including experimental error are shown in figure S8. (I to L) Tensor factorization of the signaling responses to R38Q/H16N and bitargeted variants. Experimental pSTAT5 measurements are arranged in a tensor according to the duration of treatment, ligand used, cytokine concentration, and cell type. The results of decomposition of this tensor are shown as percent variance reconstructed (R2X) versus the number of components used (I), component values representing cell type specificity (J), component values for each IL-2 mutant (K), and component values representing the effect of concentration (L).

FIGS. 7A-J show the receptor quantification and gating of PBMC-derived immune cell types. (A and B) Gating for fixed Tₕₑₗₚₑᵣ and T_{reg} cells from donor PBMCs during pSTAT5 quantification. (C and D) Fixed CD8+ T cell and NK cell gating. (E and F) Gating for live Tₕₑₗₚₑᵣ and T_{reg} cells during receptor quantification. (G) Live cell NK and NK^{bright} cell gating. (H) Live cell CD8+ cell gating. (I) Receptor quantification for each cell type described in (A to H). Experiments in (A to I) were performed using PBMCs from one donor in quadruplicate (N=4). (J) IL2Rα and IL2Rβ abundances on IL-2Rα high and low T_{reg} and Tₕₑₗₚₑᵣ populations. Cells were binned using three evenly logarithmically spaced separations between 5^{th} and 95^{th} percentile of IL-2Rα abundance.

FIGS. 8A-8D show that the concentration of optimum ligand selectivity is a function of IL2Rα affinity. (A to D) Location of the concentration at which the ratio of T_{reg} to NK (A), NK^{bright} (B) CD8⁺ (C), and Tₕₑₗₚₑᵣ (D) cell activity, assessed as pSTAT5 abundance, is maximized vs. IL2Rα affinity. Lines were fit to monovalent (thin) and bivalent (thick). Ratios were measured using cells from one donor, and signaling assays were conducted in triplicate (N=3). Affinity of IL-2 mutants was measured using biolayer interferometry, and experiments were conducted in duplicate (N=2).

FIGS. 9A-9D show that the linear and non-linear classification algorithms identify IL2Rα as most unique marker on T_{reg}s. (A and B) Largest marker coefficients determined by fitting a RIDGE-classifier to previously published CITE-seq surface marker data (A) and mRNA data (B) (GSE164378). Model was fit to identify T_{reg}s using a one-vs.-all approach. (C and D) Largest T_{reg} identification accuracies of Support Vector Classifier fit using IL2Rβ and one other marker using surface marker (C) and RNA (D) data. Accuracy is reported as Balanced Accuracy.

FIG. 10 shows the full panel of predicted versus experimental immune cell-type responses to monomeric and dimeric IL-2 muteins. Dots represent flow cytometry measurements and lines represent pSTAT response predicted by model. Experimental pSTAT measurements are shown for 0.5- and 1-hour timepoints (inset legend, top left). Predictions and experiments are shown for T_{reg}s, Tₕₑₗₚₑᵣs, NK, NK^{bright}, and CD8 cells. Each individual point is representative of one experimental replicate; experiments were conducted in triplicate (N=3).

FIG. 11 shows the full panel of predicted versus experimental IL-2Rα high, medium, and low T_{reg} and Tₕₑₗₚₑᵣ responses to monomeric and dimeric IL-2 muteins. Dots represent flow cytometry measurements and lines represent pSTAT response predicted by model. Experimental pSTAT measurements are shown for 0.5- and 1-hour timepoints. Predictions and experiments are shown for T_{reg}s, Tₕₑₗₚₑᵣs binned by their IL-2Rα abundances. Each individual point is representative of one experimental replicate; experiments were conducted in triplicate (N=3).

FIG. 12 shows a Western blot of multivalent IL-2 constructs. Western blot of monovalent, bivalent, tetravalent R38Q/H16N (lanes 1-3 and 6-8) and bivalent and tetravalent Bitargeted IL-2 (lanes 4-5 and 9-10). Samples in lanes 1-5 were run in reducing buffer, and lanes 6-10 were run in non-reducing buffer. Blot was stained with a primary anti-human-IL-2 antibody. Blots are representative of 2 experiments.

FIG. 13A-13I shows the full panel of predicted and experimental responses to R38Q/H16N multivalent mutants. (A to E) Responses of human T_{reg}s (A), Tₕₑₗₚₑᵣ S (B), NK (C), NK^{bright} (D), and CD8+ (E) cells, measured by STAT5 phosphorylation, in response to varying dosages of R38Q/H16N in various valency formats. Cells were stimulated with cytokine for 30 minutes and signaling was normalized to the largest signaling response for each cell type and donor. Points are representative of experimental results (N=5), error bars represent experimental standard deviation, and lines represent model-predicted responses. Shaded regions are indicative of the standard error of prediction when the scalar factor converting between signaling complexes and MFI was fit to multiple experiments. (F to I) Ratio of STAT5 phosphorylation in T_{reg}s to Tₕₑₗₚₑᵣ S (F), NK cells (G), NK^{bright} (H), and CD8+ (I) cells at varying dosages for R38Q/H16N in various valency formats. Dots are representative of mean of biological replicates. Responses in PBMCs from 5 donors were included, and two experimental replicates were conducted for each donor (N=5); error bars represent experimental standard deviation.

FIG. 14A-14I shows the full panel of predicted and experimental responses to bitargeted multivalent mutants. (A to E) Responses of human T_{reg}s (A), Tₕₑₗₚₑᵣ S (B), NK (C), NK^{bright} (D), and CD8+ (E) cells, measured by STAT5 phosphorylation, in response to varying dosages of Live/Dead IL-2 in various valency formats. Cells were stimulated with cytokine for 30 minutes and signaling was normalized to the largest signaling response for each cell type and donor. Points are representative of experimental results (N=5), error bars represent experimental standard deviation, and lines represent model-predicted responses. Shaded regions are indicative of the standard error of prediction when the scalar factor converting between signaling complexes and MFI was fit to multiple experiments. (F to I) Ratio of STAT5 phosphorylation in T_{reg}s to Tₕₑₗₚₑᵣ S (F), NK cells (G), NK^{bright} (H), and CD8+ (I) cells at varying dosages for bitargeted in various valency formats. Dots are representative of mean of biological replicates. Responses in PBMCs from 5 donors were included, and two experimental replicates were conducted for each donor, error bars represent experimental standard deviation.

FIG. 15A-15H shows the receptor quantification and gating of PBMC-derived immune cell types. (A to E) Gating for fixed ILC2s and T_{reg} cells from donor PBMCs. (F and G) Mean fluorescent intensity (MFI) for T_{reg}s and ILC2s across aggregated across 4 donors. (H) MFI of CD25 on T_{reg}s and ILC2s for each donor. In (A to H), one experimental replicate was performed for each of 4 donors.

### DETAILED DESCRIPTION

The present disclosure is directed generally to multispecific multivalent biomolecules that comprise two or more covalently linked cytokines or variants thereof, where at least one cytokine is signaling competent and one cytokine is signaling deficient. The signaling competent and signaling deficient cytokines may be the same cytokine (e.g., both IL-2, one signaling competent and one signaling deficient), or they may be two different cytokines (e.g., signaling-competent IL-7 and signaling deficient IL-2). In addition to at least one signaling-competent cytokine and one signaling-deficient cytokine in the biomolecule, additional cytokines (the same or different, signaling-competent or signaling deficient, or both) may be present. The signaling competent and signaling deficient cytokines may be provided as a fusion polypeptide, e.g., expressed as a single polypeptide chain. In other embodiments, each of the signaling competent cytokine and the signaling deficient cytokine may be cross-linked, such as by disulfide crosslinking of Fc sequences comprising each cytokine. Such preceding description generally described variations in the multispecific multivalent biomolecules disclosed herein and non-limiting examples will be described below.

In some aspects, the at least two cytokines comprising the multispecific multivalent biomolecules disclosed herein are the same or different chemokine, interferon, interleukin, lymphokine, or tumor necrosis factors. In some aspects, the at least two cytokines comprising the multispecific multivalent biomolecules disclosed herein are the same or different interleukins. In some aspects, the at least two cytokines comprising the multispecific multivalent biomolecules disclosed herein are the same or different common γ-chain (γ_{c}) receptor cytokines. While the description and examples provided herein are mainly on multispecific multivalent biomolecules comprising the same or different common γ-chain (γ_{c}) receptor cytokines, the disclosure is not so limiting and multispecific multivalent biomolecules comprising other cytokines are fully embraced herein.

The terms homodimeric or heterodimeric, or syntactical variants thereof, are used to refer to the multispecific multivalent biomolecules disclosed herein that comprise two of the same, or two different, respectively, dimerizable Fc-containing molecules cross-linked together. Non-limited examples of homodimeric biomolecules are two SEQ ID NO:7 molecules, two SEQ ID NO: 20 molecules, two SEQ ID NO: 22 molecules, two SEQ ID NO:24 molecules, two SEQ ID NO:29 molecules, two SEQ ID NO:31 molecules or two SEQ ID NO: 35 molecules forming a tetrameric biomolecule. In some embodiments, two different crosslinkable molecules form a heterodimeric tetramer. Non-limiting examples of heterodimeric biomolecules include a combination of SEQ ID NO:7 and SEQ ID NO:20, and a combination of SEQ ID NO:22 and SEQ ID NO:35. The term tetravalent refers to a multispecific multivalent biomolecule comprising two bivalent biomolecules. The term bivalent refers to a biomolecule having two cytokines with the same or different specificities, such as a native (wild type or WT) IL-2 and a signaling deficient IL-2. Such bivalent biomolecules may comprise a single chain polypeptide comprising both cytokines, or may comprise a cross-linked biomolecule formed by cross-linking two single-chain polypeptides, each with a cytokine and a means for cross-linking to the other, such as a dimerizable Fc region. The term multispecific refers to a biomolecule as disclosed herein with at least two different specificities, such as a native IL-2 and a signaling deficient IL-2.

The common γ-chain (γ_{c}) receptor cytokines, such as interleukin (IL)-2, 4, 7, 9, 15, and 21, are integral for modulating both innate and adaptive immune responses. The common γ-chain receptor cytokines are promising immune therapies due to their central role in coordinating the proliferation and activity of various immune cell populations. One of these cytokines, interleukin (IL)-2, has potential as a therapy in autoimmunity but is limited in effectiveness by its modest specificity toward regulatory T cells (T_{regs}). IL-2 muteins with altered receptor-ligand binding kinetics can improve the cell type selectivity of the signaling response. Furthermore, therapeutic ligands are often made dimeric as antibody Fc fusions to confer desirable pharmacokinetic benefits, with unexplored signaling consequences. The therapeutic potential and complexity of this cytokine family make computational models especially valuable for rational engineering. IL-2 is an approved, effective therapy for metastatic melanoma, and the antitumor effects of IL-2 and IL-15 have been explored in combination with other treatments.

To address the limitations of natural ligands, engineered proteins have been produced with potentially beneficial properties. For example, mutants skewed toward IL-2Rα over IL-2Rβ binding selectively expand T_{reg} populations over cytotoxic T cells and NK cells as compared to native IL-2. Nonetheless, understanding these cytokines' regulation is stymied by their complex binding and activation mechanism. Any intervention imparts effects across multiple distinct cell populations, with each population having a unique response defined by its receptor expression.

The present disclosure provides compounds and mechanisms for deriving cell type-selective cytokine responses. Heretofore, altered cytokine selectivity has almost entirely been derived through changes in affinity toward different receptors. For instance, T_{reg} have a higher abundance of IL2Rα. Retaining the high affinity for this receptor, while decreasing the affinity toward IL2Rβ, provides some selectivity toward T_{reg}. Multivalency can enhance selectivity. Unlike affinity changes, valency provides avidity effects, which allows one to selectively activate cells based on the quantitative abundance of a receptor, rather than based on a distinct pattern of receptor expression. Thus, one can make, for example, a tetravalent Fc fusion with four IL-2 monomer units that is more Tres selective than observed as a monomeric or bivalent Fc fusion. Such benefits of multivalency have been explored in, for example, PCT/US2022/35711. However, improvements can be made beyond the selectivity achieved through multivalency alone.

This disclosure provides for a combination of cytokine multivalency and varied cytokine selectivity therein, which has been found to enhance selectivity, increase potency, and enable the modulating of cytokine signaling that is therapeutically advantageous for addressing immunological functions useful for the treatment of numerous conditions and diseases. Such combination of multivalency and varied cytokine selectivity may be provided, by multispecific multivalent biomolecule compositions comprising at least one cytokine that is signaling competent, such as the native cytokine or a signaling competent mutein thereof, and a signaling deficient cytokine, which may be the same or different cytokine as the signaling competent cytokine, but have deficient signaling such as by mutation compared to its native cytokine. In some embodiments, the different property is an altered receptor specificity or binding property, such as increased receptor affinity, decreased receptor affinity, increased receptor signaling, decreased receptor signaling, or any combination thereof, including reduced affinity or lack of signaling. Such multispecific multivalent compositions have uses in regulation of the immune response.

As shown in the examples herein, high-throughput profiling data was used to inquire whether binding to other proteins on the T_{reg} surface would further enhance selectivity. A surprising result was that IL2Rα (CD25) was identified as a lead candidate. This is surprising because the IL-2 cytokine was allowed to vary in its affinity toward both IL2Rα and IL2Rβ, so there was presumably already IL2Rα binding. It was discovered that multivalent complexes, as a result of having multiple monomer sites, can benefit from having individual monomers of heterogeneous (e.g., multispecific) composition. Selectivity is always increased by preferring IL2Rα binding over that of IL2Rβ, but IL2Rα is not signaling competent and so a single IL-2 monomer that only binds IL2Rα would have no signaling effect. Consequently, breaking the symmetry of monomer composition leads to a great enhancement of selectivity. This additionally opens the possibility to make other, non-T_{reg}-selective cytokines T_{reg}-selective. For instance, IL-7 may be more potent in promoting T_{reg}-mediated immune suppression, but typically cannot be made to have T_{reg}-selective effects because the IL-7 receptors are not uniquely abundant on T_{reg}s. However, in one embodiment, a multispecific multivalent complex is provided having IL-7 with signaling “dead” IL-2 monomers that carry IL-7 to T_{reg} exclusively.

Thus, the present disclosure provides a multispecific multivalent biomolecule comprising two or more same or different covalently linked cytokines, wherein at least one cytokine is a signaling-competent cytokine or a signaling-competent variant thereof; and the second cytokine or variant thereof is a same or different cytokine from the first cytokine, and is signaling-deficient compared to that of a native second cytokine. As will be described herein, such multispecific multivalent biomolecule may be provided in any of a number of formats that provide the at least two cytokines and for various uses as described herein. For example, the signaling-competent cytokine and signaling-deficient cytokine may be provided on a single polypeptide chain, for example a fusion polypeptide. In other embodiments, the signaling-competent cytokine and signaling-deficient cytokine may be cross-linked, such as wherein each is present on a fusion polypeptide with a cross-linkable polypeptide such as a Fc hinge region. In other variations comprising more than two cytokines, two fusion polypeptides, each with a Fc portion and one or more cytokine as described herein may be cross-linked, so as to provide at least one signaling-competent cytokine and one signaling-deficient cytokine covalently linked. The components, expression, cross-linking, and other features described for the multispecific multivalent biomolecule, methods for making them, and methods for use are not intended to be limiting.

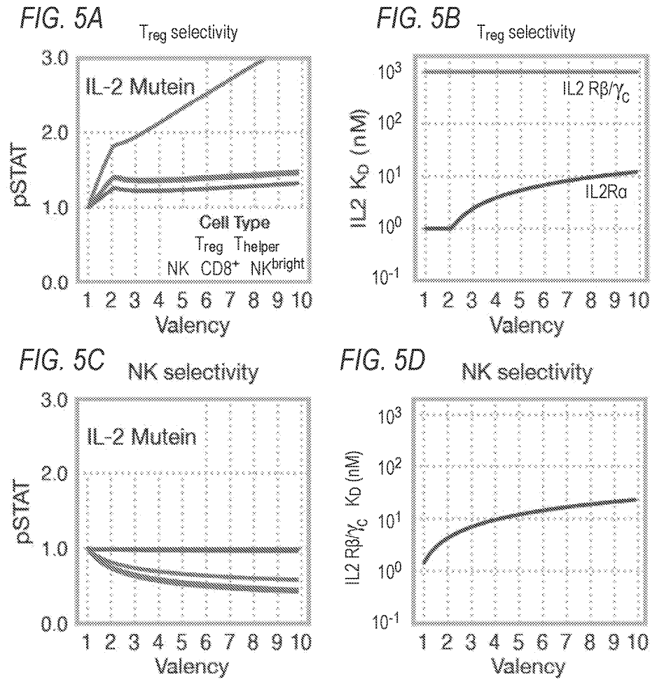

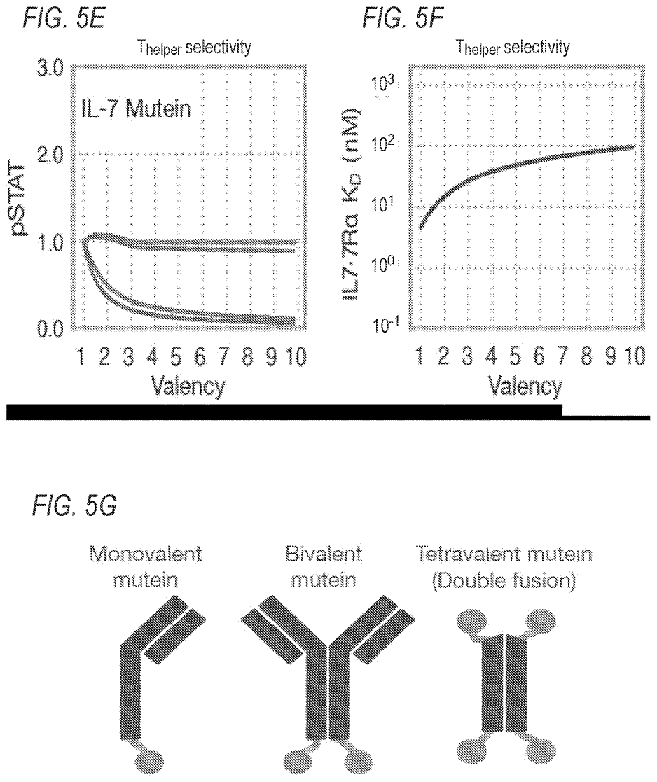

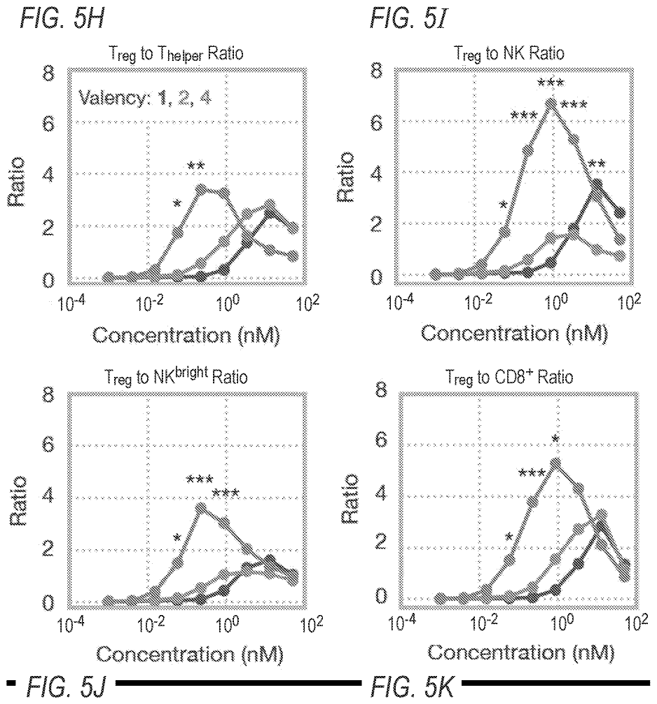

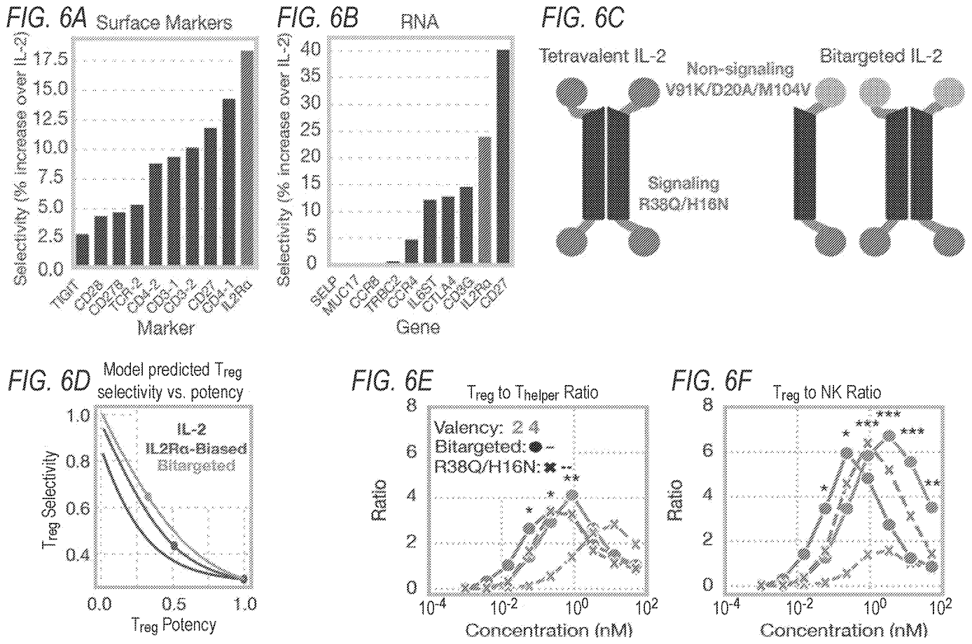

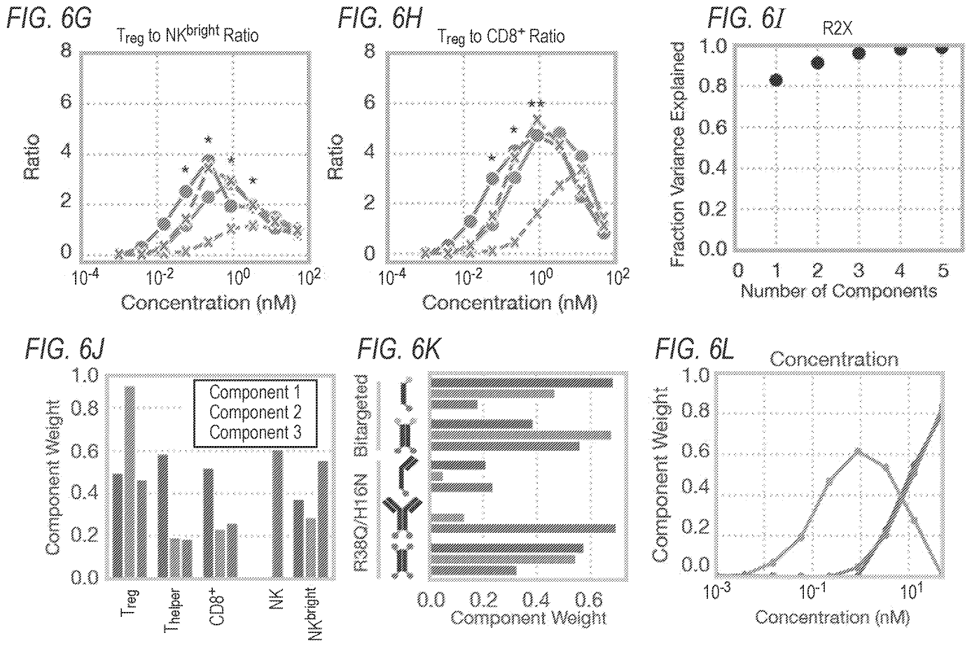

Thus, as shown herein, a systematic exploration is provided on how ligand properties determine signaling response and specificity across 13 engineered IL-2 variants. The study included clinically relevant muteins alongside variation in Fc fusion format. Dimensionality reduction in tensor form identified how ligand properties alter response, revealing that multivalent cytokines have unique specificity advantages. Using a multivalent binding model, this unique specificity was found to arise from surface binding avidity effects. Both the analysis using this model and experimental validation indicated that modulating the valency of cytokines may offer T_{reg} selectivity far beyond that achievable through affinity modulation alone (FIG. 5), and demonstrated this strategy experimentally by expressing tetravalent IL-2 fusions with greater T_{reg} selectivity than current state-of-the-art monovalent or bivalent affinity muteins. Finally, it was uncovered that IL2Rα itself is the optimal target for designing T_{reg}-selective binding, and that cis-targeting can be designed into multivalent IL-2 fusions through asymmetric tetravalent IL-2 fusions, again improving on signaling selectivity (FIG. 6). In total, these results show that not only do multivalency and cis targeting of IL2Rα improve T_{reg} selectivity, but that these paired strategies represent the only known method for overcoming the selectivity-potency tradeoff faced by T_{reg}-selective muteins.

These results have clear implications for the design of T_{reg}-directed IL-2 therapies, an area of enormous interest for the treatment and management of autoimmune diseases. It was shown computationally and experimentally that multivalency and bitargeting can enhance IL-2 T_{reg} selectivity for potential use in clinical settings, where IL-2 based therapies have traditionally struggled. Engineering valency requires precise compensatory adjustments in the ligand affinity; given that experiments were limited to pre-existing muteins, it is expected that the selectivity gains might be improved even further by identifying muteins with optimal affinities. Various T_{reg} selective affinity mutants continue to be published and many previously developed affinity mutants were not included in our analysis—as shown herein, paired affinity and valency engineering confers selectivity beyond what is achievable in monovalent formats of any affinity; the approach here will synergize with the continued development of affinity variants. The multivalent and bitargeted designs will be tested in vivo to show that these selectivity gains translate to the in vivo setting. In the majority of previous studies, the selectivity with which pSTAT5 activity is induced in T_{reg}s has consistently translated to selective expansion of T_{reg}s in in vivo, and thus supporting the mutein's heightened selectivity translating to such settings. T_{reg} selectivity is central to the mechanism of action for these therapies, and so it is expected that these benefits to selectivity will improve therapeutic properties in several ways: more potent activation of signaling in T_{reg}s without off-target effects may improve the potency of these therapies and the breadth of applications; reduced toxicity may allow for more routine use with minimal patient monitoring. The superior selectivity offered by engineered multivalent ligands will likely further increase their in vivo pharmacokinetic lifetimes, in turn requiring less frequent dosing, as most drug clearance occurs via receptor-mediated endocytosis in off-target populations.

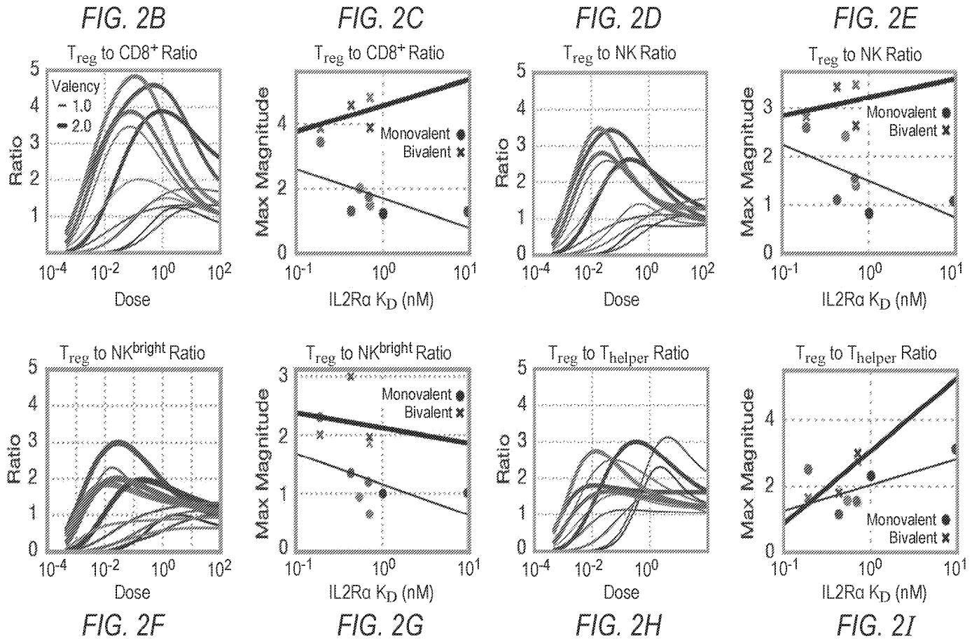

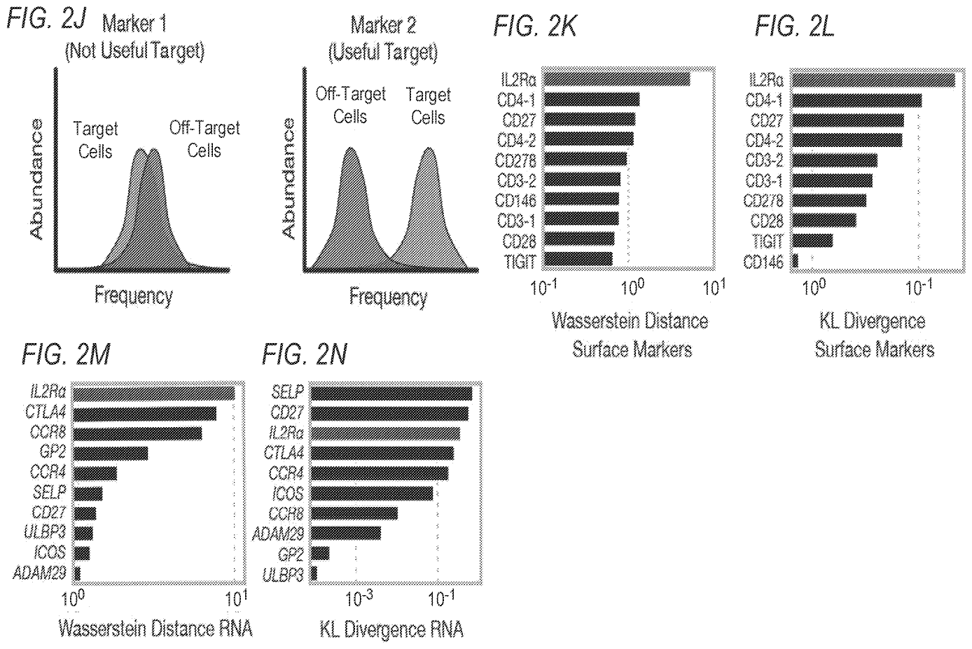

Heterospecificity, in this case exploited through bitargeting, opens a whole range of new possibilities through its ability to decouple the targeting and signaling properties of cytokine therapy and/or combine synergistic signals. This capability has been demonstrated through bispecific antibodies previously, and through the design of cis-targeted cytokine-antibody fusions. However, we showed that, unlike other immune cells, T_{reg}s do not express any surface marker more selective than IL2Rα (FIG. 2, I to L). Consequently, there are no alternative targeting options to our approach of using the IL-2 receptors themselves (FIG. 6). Beyond these results, heterospecificity creates opportunities for synergistic receptor agonism. For example, PD-1 cis-targeting with IL-2 increases the stemness of CD8⁺ T cells and consequently their tumor killing capacity. While IL-2 has been employed as a therapy because of its T_{reg} selectivity, there is no reason to believe that the cytokine's signaling effects are optimal for enhancing T_{reg} suppressive activities. In fact, with cell therapies, where selectivity is not a concern and non-natural cytokine receptors can be introduced, other cytokine signaling such as IL-9 is qualitatively more effective than IL-2 at promoting cytotoxic T cell function. Thus, one possibility enabled by bitargeting is potentially plug-and-play combinations of one or more cytokines that are more capable than IL-2 of driving desirable T_{reg} properties, made T_{reg}-selective through their fusion to multivalent IL2Rα-targeting complexes. More systems-level research into the signaling regulation of T_{reg} proliferation and suppressive activities, and comparisons to other cytokines beyond IL-2, is needed to develop these possibilities. Such studies, which will be used to not only identify the optimal signal using functional suppressive assays, but also to further improve the selectivity with which that signal is delivered, will justify the translation of such fusion proteins into in vivo disease model studies.

### Cytokines

Cytokines include chemokines, interferons, interleukins, lymphokines, bone morphogenetic protein and tumor necrosis factors. Non-limiting examples include interleukins such as the common γ-chain receptor cytokines (discussed in more detail below), transforming growth factor β (TGF-β) and transforming growth factor α (TGF-α).

### Common γ-Chain Receptor Cytokines

Examples of common γ-chain receptor cytokines include, but are not limited to, IL-2, IL-4, IL-7, IL-9, IL-15 and IL-21.

The ensuing description of multispecific multivalent biomolecule will use common γ-chain receptor cytokines as examples of the various signaling-competent and signaling-deficient components, as well as various types of constructs (fusion polypeptides, fusion polypeptides with cross-linkable sequences, cross-linked fusion polypeptides with cross-linkable sequences, etc.). However, the disclosure is not intended to be at all limited to such examples and one of skill in the art will readily fashion other multispecific multivalent biomolecule guided by the teachings herein.

### Signaling-Competent Cytokines

Signaling-competent cytokines include native cytokines as well as muteins and other variants thereof that retain signaling activity similar to that of the native cytokine. Signaling competent cytokines may or may not have the N-terminal signal sequence (e.g., amino acids 1-20 of IL-2).

Native cytokines. The compositions disclosed herein comprise at least one signaling-competent cytokine, such as a native common γ-chain receptor cytokine. Examples of common γ-chain receptor cytokines include, but are not limited to, IL-2, IL-4, IL-7, IL-9, IL-15 and IL-21. One of ordinary skill in the art would reasonably recognize that the principles and methodologies disclosed herein are applicable not only to these interleukins, but also to other members of the common γ-chain receptor cytokine family, both currently known or discovered in the future. In other embodiments, cytokines as generally described herein may be a component of the compositions disclosed herein. Non-limiting examples include TGF-β and TGF-α. In other embodiments, bone morphogenetic proteins are embraced herein.

Native cytokine signaling-competent variants. A signaling-competent variant of a native cytokine as used herein refers to a cytokine that has a modification that does not alter its signaling properties. Non-limiting examples of such native cytokines include IL-2 with a R38Q, H16N or both R38Q/H16N mutation (SEQ ID NO:28) (see Shen et al., Front. Immuol. 8 May 2020; 11:832); IL-4 with a R121Q or both R121K/Y124F mutation (SEQ ID NO:21) (Junttila et al., Nature Chem. Biol. 8:990-998 (2012); and IL-21 with a R76E mutation (Shen et al., op. cit).

### Signaling-Deficient Cytokines

The compositions herein comprising a signaling-deficient common γ-chain receptor cytokine comprise at least one common γ-chain receptor cytokine with deficient signaling as compared to its native cytokine. For example, a signaling-deficient IL-2 may have mutations V91K, D20A, M104V, or any combination thereof. In one embodiment, the signaling-deficient IL-2 has V91K, D20A and M104V. Other examples include IL-4 R121D/Y124D (Mueller et al., 2002, Biochim Biophys Acta 1592(3):237-250), which lacks interaction with the γ-chain receptor while retaining binding affinity for IL-4Rα; IL-21 Q116D/H120D or Q116D/L123D (Xu et al., 2022, J Biol Chem 285(15):12223-12231), which lack interaction with the γ-chain receptor but retain binding affinity for IL-21Rα; and IL-15 Q101D/Q108D (Kim et al., 198, J Immunol 160(12): 5742-5748), which lacks interaction with the γ-chain receptor but retain binding affinity for IL-15Rα. In some embodiments, lack of or substantially reduced signaling may be referred to as dead.

The at least one signaling-deficient cytokine may be the cytokine as the signaling-competent cytokine in the multispecific multivalent biomolecule, or it may be a different cytokine. By way of non-limiting example, such compositions with the same cytokine include a composition comprising at least a signaling-competent IL-2 and at least a signaling-deficient IL-2, or a signaling-competent IL-7 and at least a signaling-deficient IL-7. By way of non-limiting example, such compositions with different types of cytokine include a composition comprising at least a signaling-competent IL-7 and at least a signaling-deficient IL-2. As noted herein, the signaling-competent cytokine may be any native cytokine or a variant that is signaling-competent; the signaling-deficient cytokine may be a mutein of any native cytokine. Other non-limiting examples include native or a signaling-competent TGF-β and a signaling-deficient IL-2, and native or a signaling-competent TGF-α and a signaling-deficient IL-2.

In one embodiment, the disclosed multispecific multivalent biomolecules comprising a signaling-deficient common γ-chain receptor cytokine, or a variant thereof, has lowered affinity for the cognate γ-chain private receptor as compared to the same common γ-chain private receptor cytokine in native form. In one embodiment, the affinity is lowered at least 2-fold. In some embodiments the affinity for the private receptor is lowered by more than or equal to about 2-fold, more than or equal to about 5-fold, more than or equal to about 10-fold or more than or equal to about 50-fold, compared to affinity the native cytokine or a signaling-competent variant thereof.

In one embodiment, the disclosed multispecific multivalent biomolecules comprising signaling-deficient common γ-chain receptor cytokines, or a variant thereof, has lowered signaling of the cognate γ-chain family receptor as compared to the same common γ-chain receptor cytokine in native form. In one embodiment, the signaling activity is lowered at least or equal to about 10-fold, at least or equal to about 50-fold, at least or equal to about 100-fold, at least or equal to about 1000-fold, or signaling is substantially eliminated. In one embodiment, the disclosed multispecific multivalent biomolecules comprising signaling-deficient common γ-chain receptor cytokines, or a variant thereof, has lowered signaling of the private receptor as compared to the same common γ-chain receptor cytokine in native form. In one embodiment, the signaling activity is lowered at by least 2-fold, by at least 5-fold, by at least 10-fold, at least 50-fold, at least 100-fold, at least 1000-fold, or substantially eliminated. Signaling deficiency may be assessed by methods such as those described herein below.

Such signaling-deficient cytokines may have reduced receptor affinity as compared to the native cytokine. In one embodiment, the disclosed multispecific multivalent biomolecules comprising signaling-deficient common γ-chain receptor cytokines, or a variant thereof, has lowered affinity of the cognate γ-chain family receptor as compared to the same common Y-chain receptor cytokine in native form. In one embodiment, the affinity is lowered at least or equal to about 10 fold, at least or equal to about 50 fold, at least or equal to about 100 fold, at least or equal to about 1000 fold, or affinity is substantially eliminated. In one embodiment, the disclosed multispecific multivalent biomolecules comprising signaling-deficient common γ-chain receptor cytokines, or a variant thereof, has lowered affinity for the private receptor as compared to the same common γ-chain receptor cytokine in native form. In one embodiment, the affinity is lowered at least 10 fold, at least 50 fold, at least 100 fold, at least 1000 fold, or substantially eliminated. Signaling deficiency may be assessed by methods such as those described herein below.

In any embodiment herein, the common γ-chain (γ_{c}) receptor cytokines, such as interleukin (IL)-2, 4, 7, 9, 15, and 21, or a variant thereof, may comprise one or more modifications, such as but not limited to an amino acid modification such as an amino acid substitution, insertion, and/or deletion; truncation; modification of a (free) N- or C-terminus; and/or a post-translational modification such as but not limited to glycosylation, acylation, phosphorylation, deamidation, pegylation or sulphation. As noted herein, such modifications may alter or not alter the receptor interaction property; such alterations that do not alter the receptor interaction property are considered for the purposes herein a signaling competent cytokine. Also as noted herein, such modifications that alter the receptor interaction and/or affinity and/or signaling property are considered for the purposes herein a signaling deficient cytokine. A non-limiting example of such signal competent muteins are IL-2 muteins with R38Q and/or H16N mutations; numerous other muteins of the common γ-chain receptor cytokines comprising the multivalent cytokines disclosed herein are known in the art and are embraced herein. Another example is IL-2 superkine (SEQ ID NO:01) that may be used in the IL-2-comprising multivalent cytokines disclosed herein. Silva et al., 2019, De novo design of potent and selective mimics of IL-2 and IL-15, Nature 565:186-191, describe other modified forms of IL-2 as well as IL-15, such as neoleukin-2/15 (Neo-2/15), that may be used in the multivalent cytokines disclosed herein. Such modifications in one embodiment enhance the biological activity, receptor binding activity, receptor affinity, receptor avidity, half-life, resistance to degradation, resistance to metabolism, resistance to proteolysis, and/or other features that modify and/or improve one or more features of the multivalent cytokines disclosed herein for clinical use, dosing, effective and/or convenient dosing regimen, administration, storage, stability, ease of manufacturing, or other factors, in any combination. Any such modification may also be provided on fragments of the common γ-chain receptor cytokines disclosed herein, which retain their activity for the purposes described herein and hence referred to immunologically active or altered fragments.

Non-limiting examples of IL-2 muteins with signaling deficient properties include V91K, D20A, M104V or any combination thereof, including V91K, D20A and M104V. Non-limiting examples of IL-4 muteins with signaling deficient properties include R121D/Y124D. Non-limiting examples of IL-15 muteins with signaling deficient properties include Q101D/Q108D. Non-limiting examples of IL-21 muteins with signaling deficient properties include Q116D/H123D.

### Multispecific Multivalent Biomolecule Constructs: General Principles

Various constructs of the multispecific multivalent biomolecules disclosed herein are possible and embodied herein, such as wherein (1) the signaling-competent and signaling-deficient cytokines are on the same polypeptide chain; (2) the signaling-competent and signaling-deficient cytokines are on different polypeptide chains and cross-linked; and (3) the signaling-competent and signaling-deficient cytokines are on the same polypeptide chain and cross-linked to another cytokine. Any of the foregoing constructs may have one or more additional cytokines. Such constructs are merely exemplary of ways to construct the multispecific multivalent biomolecules disclosed here and others are embraced herein.

In one embodiment, the multivalent biomolecule disclosed herein comprises at least 2, at least 3, at least 4, at least 5 or at least 6 common γ-chain receptor cytokines variants thereof, wherein at least one cytokine or variant thereof is signaling competent and at least one cytokine or variant thereof is signaling deficient, as described herein. In one embodiment, the multivalent biomolecule disclosed herein comprises 2, 3, 4, 5 or 6 common γ-chain receptor cytokines or variants thereof, at least one cytokine or variant thereof is signaling competent and at least one cytokine or variant thereof is signaling deficient, as described herein. Single-chain polypeptides having means for cross-linking with another single-chain polypeptide (the same or different) allow for the opportunity to provide a composition disclosed herein wherein the one or more signaling-competent cytokine is on one of the cross-linkable single-chain polypeptides and a signaling-deficient cytokine is on the other; or wherein each of the cross-linkable polypeptides has at least one signaling-competent and at least one signaling-deficient cytokine. Such variations are embraced among the multispecific multivalent biomolecules disclosed herein.

In other embodiments, the multispecific multivalent biomolecules disclosed herein may comprise at least any signaling-competent cytokine, such as TGF-β or TGF-α, and at least any signaling-deficient cytokine such as a signaling-deficient IL-2 mutein as described herein. As noted herein, such multispecific multivalent biomolecule may be a single-chain polypeptide comprising at least both the signaling-competent and signaling-deficient cytokine, or at least each such component may be provided in the multispecific multivalent biomolecule by cross-linking separate single-chain polypeptide chains.

In some embodiments, the multispecific multivalent biomolecules disclosed herein further comprise a cross-linkable polypeptide or other moiety, such that two such same or different polypeptides can be cross-linked to form a multispecific multivalent biomolecule. One non-limiting example of a cross-linkable polypeptide that can be expressed in a single-chain polypeptide with a cytokine described herein is the Fc hinge region CH2/CH3, without a C-terminal K, from human IgG Fc, such as a Fc from IgG1 (SEQ ID NO:03), IgG2, IgG3 or IgG4. One of ordinary skill in the art would readily use generally known techniques to construct the Fc fusion proteins disclosed herein. Such Fc hinge region provides means for cross-linking two single-chain polypeptides comprising Fc hinge regions, via disulfide links. Such Fc hinge region also provides for an improved in vivo half-life. Such cross-linked multispecific multivalent biomolecules are described herein. Such Fc hinge region CH2/CH3 without a C terminal K may be referred to herein simply as Fc when describing the components of a single chain polypeptide or any constructs described herein.

The multispecific multivalent biomolecules disclosed herein are readily manufacturable using methods known in the art. For single-chain polypeptide expression, methods for cellular and acellular expression systems are known in the art, and methods for scale-up, preparation and purification of recombinant proteins for clinical use are well established. Methods for dimerization or oligomerization of the polypeptides described herein, using bifunctional cross-linking agents, or disulfide crosslinking of Fc portions of polypeptides as described herein, are also known in the art. By way of non-limiting example, Fc regions of human IgG1 dimerize by disulfide formation at cysteines 109 and 112 (numbering based on the full heavy chain polypeptide, gene IGHG1). In the Fc sequence of SEQ ID NO:03, cysteines at positions 6 and 9 are involved in dimerization.

Other IgG isotypes form interchain disulfide cross-links at positions well known in the art.

### Multispecific Multivalent Biomolecule Constructs: Single Chain Polypeptides

In one embodiment, at least one signaling-competent common γ-chain receptor cytokine, or variant thereof, and at least one signaling-deficient cytokine, or a variant thereof, are expressed as a single-chain polypeptide. Such single-chain polypeptides can be generated following standard molecular biology techniques. For example, multiple same or different units of the common γ-chain receptor cytokines, at least one signaling-competent and at least one signaling-deficient, can be multimerized optionally with linkers generally known in the art, such as (G₄S)₄. Such single-chain polypeptides comprising at least one signaling-competent cytokine or variant thereof and one signaling-deficient cytokine or variant thereof are thus covalently linked by residing on the same polypeptide chain. In one embodiment, the single polypeptide chain may comprise one or more additional signaling-competent cytokines or variant thereof and/or one or more signaling-deficient cytokines or variant thereof. In other embodiments, the single polypeptide chain may comprise an Fc polypeptide, such as the IgG1 Fc hinge region CH2/CH3, without C-terminal K. Such Fc hinge region and variations thereof confer various properties such as but not limited to allowing disulfide crosslinking between Fc regions on different polypeptides, or for improved in vivo half-life, or other purposes as described herein. Such use of the Fc region for cross-linking is described further below. In one embodiment, a non-cross-linkable (“monovalent Fc fragment”) Fc hinge region has the sequence DKTHTCPPCPAPELLGGPSVFLFPPKPKDTLMISRTPEVTC VVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVNLTCLVKGF YPSDIAVEWESNGQPENNYKTTPPVLDSDGSFFLNSTLTVDKSRWQQGNVFSCSVM HEALHNHYTQKSLSLSP (SEQ ID NO: 02). In another embodiment, a cross-linkable (“bivalent Fc fragment”) Fc region has the sequence DKTHTCPPCPAPELLGGPSVFLFPPKPKDTLMISR TPEVTCVVVDVSHEDPEVKFNWYVDGVEVHNAKTKPREEQYNSTYRVVSVLTVLH QDWLNGKEYKCKVSNKALPAPIEKTISKAKGQPREPQVYTLPPSRDELTKNQVSLTC LVKGFYPSDIAVEWESNGQPENNYKTTPPVLDSDGSFFLYSKLTVDKSRWQQGNVFS CSVMHEALHNHYTQKSLSLSP (SEQ ID NO: 03).

In some embodiments, the signaling-deficient cytokine may not include the N-terminal signal sequence (e.g., amino acids 1-20), may comprise one or more muteins, or both. In one embodiment, a signaling-deficient IL-2 excludes amino acids 1-20 of native IL2 with muteins V91K, D20A and M104V: APTSSSTKKTQLQLEHLLLALQMILNGINNYKNPKLTRML TFKFYMPKKATELKHLQCLEEELKPLEEVLNLAQSKNFHLRPRDLISNINKIVLELKGS ETTFVCEYADETATIVEFLNRWITFCQSIISTLT (SEQ ID NO:04).

To produce such single-chain polypeptides comprising cytokines and optional linkers and/or Fc regions, such polypeptides may be expressed using the Expi293 expression system according to manufacturer instructions (e.g., Thermo Scientific). In some embodiments, proteins were expressed as human IgG1 Fc fused at the N or C terminus to the human cytokine or variant sequence through a (G₄S)₄ linker. C-terminal fusions may omit the C-terminal lysine residue of human IgG1. Proteins may be purified using MabSelect resin (GE Healthcare). Proteins may be biotinylated using BirA enzyme (BPS Biosciences) according to manufacturer instructions, and extensively buffer-exchanged into phosphate buffered saline (PBS) using Amicon 10 kDa spin concentrators (EMD Millipore).

The following table sets forth the sequences used in the polypeptides disclosed herein, and non-limiting examples of single-chain polypeptides comprising a signaling-competent cytokine or variant thereof and one signaling-deficient cytokine or variant thereof. Some examples include a dimerizable Fc region; others do not.

| SEQ ID NO | |
|---|---|
| Description | Sequence |
| SEQ ID NO: 01 | DLISNINVIVLELKGSETTFMCEYADETATIVEFLNRWITFCQSIISTLT (SEQ |
| IL-2 superkine | ID NO: 01) |
| SEQ ID NO: 02 | DKTHTCPPCPAPELLGGPSVFLFPPKPKDTLMISRTPEVTC |
| Non-crosslinkable | VVVDVSHEDPEVKFNWYVDGVEVHNAKTKPREEQYNSTYRVVSVL |
| Fc hinge region | TVLHQDWLNGKEYKCKVSNKALPAPIEKTISKAKGQPREPQVYTLPP |
| | SRDELTKNQVNLTCLVKGFYPSDIAVEWESNGQPENNYKTTPPVLDS |
| | DGSFFLNSTLTVDKSRWQQGNVFSCSVMHEALHNHYTQKSLSLSP |
| | (SEQ ID NO: 02) |
| SEQ ID NO: 03 | DKTHTCPPCPAPELLGGPSVFLFPPKPKDTLMISR |
| Cross-linkable Fc | TPEVTCVVVDVSHEDPEVKFNWYVDGVEVHNAKTKPREEQYNSTY |
| fragment | RVVSVLTVLHQDWLNGKEYKCKVSNKALPAPIEKTISKAKGQPREP |
| | QVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQK |
| | SLSLSP (SEQ ID NO: 03) |
| SEQ ID NO: 04 | APTSSSTKKTQLQLEHLLLALQMILNGINNYKNPKLTRML |
| Signaling deficient | TFKFYMPKKATELKHLQCLEEELKPLEEVLNLAQSKNFHLRPRDLIS |
| IL-2 (V91K, D20A, | NINKIVLELKGSETTFVCEYADETATIVEFLNRWITFCQSIISTLT (SEQ ID |
| M104V) excluding | NO: 04) |
| signal sequence, | |
| amino acids 1-20 | |
| SEQ ID NO: 05 | MYRMQLLSCIALSLALVTNSAPTSSSTKKTQLQLEHLLLDLQMILNGINNYK |
| WT IL-2 (with | NPKLTRMLTFKFYMPKKATELKHLQCLEEELKPLEEVLNLAQSKNFHLRPR |
| signal sequence) | DLISNINVIVLELKGSETTFMCEYADETATIVEFLNRWITFCQSIISTLT (SEQ |
| | ID NO: 05) |
| SEQ ID NO: 06 | GGGGSGGGGSGGGGSGGGGS (SEQ ID NO: 06) |
| (G₄S)₄ linker | |
| SEQ ID NO: 07 | MYRMQLLSCIALSLALVTNSAPTSSSTKKTQLQLEHLLLDLQMILNGINNYK |
| Tetravalent | NPKLTRMLTFKFYMPKKATELKHLQCLEEELKPLEEVLNLAQSKNFHLRPR |
| bitargeted | DLISNINVIVLELKGSETTFMCEYADETATIVEFLNRWITFCQSIISTLT |
| IL-2 and signaling | GGGGSGGGGSGGGGSGGGGS |
| deficient IL-2 | DKTHTCPPCPAPELLGGPSVFLFPPKPKDTLMISRTPEVTCVVVDVSHEDPE |
| Native IL-2 | VKFNWYVDGVEVHNAKTKPREEQYNSTYRVVSVLTVLHQDWLNGKEYK |
| (G₄S)₄ | CKVSNKALPAPIEKTISKAKGQPREPQVYTLPPSRDELTKNQVSLTCLVKGF |
| Dimerizing Fc | YPSDIAVEWESNGQPENNYKTTPPVLDSDGSFFLYSKLTVDKSRWQQGNVF |
| (G₄S)₄ | SCSVMHEALHNHYTQKSLSLSP |
| IL-2(21-153; V91K, | GGGGSGGGGSGGGGSGGGGS |
| D20A, M104V) | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 07) |
| | Comprising: |
| | MYRMQLLSCIALSLALVTNSAPTSSSTKKTQLQLEHLLLDLQMILNGINNYK |
| | NPKLTRMLTFKFYMPKKATELKHLQCLEEELKPLEEVLNLAQSKNFHLRPR |
| | DLISNINVIVLELKGSETTFMCEYADETATIVEFLNRWITFCQSIISTLT (WT |
| | IL-2; SEQ ID NO: 05) |
| | GGGGSGGGGSGGGGSGGGGS ((G₄S)₄ linker, SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 3) |
| | GGGGSGGGGSGGGGSGGGGS ((G₄S)₄ linker, SEQ ID NO: 6) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 4) |
| SEQ ID NO: 08 | MYRMQLLSCIALSLALVTNSAPTSSSTKKTQLQLEHLLLDLQMILNGINNYK |
| Bivalent bitargeted | NPKLTRMLTFKFYMPKKATELKHLQCLEEELKPLEEVLNLAQSKNFHLRPR |
| WT IL-2 and | DLISNINVIVLELKGSETTFMCEYADETATIVEFLNRWITFCQSIISTLT |
| signaling deficient | GGGGSGGGGSGGGGSGGGGS |
| IL-2 | DKTHTCPPCPAPELLGGPSVFLFPPKPKDTLMISRTPEVTCVVVDVSHEDPE |
| Native IL-2 | VKFNWYVDGVEVHNAKTKPREEQYNSTYRVVSVLTVLHQDWLNGKEYK |
| (G₄S)₄ | CKVSNKALPAPIEKTISKAKGQPREPQVYTLPPSRDELTKNQVNLTCLVKGF |
| Monovalent Fc | YPSDIAVEWESNGQPENNYKTTPPVLDSDGSFFLNSTLTVDKSRWQQGNVF |
| (G₄S)₄ | SCSVMHEALHNHYTQKSLSLSP |
| IL-2(21-153; V91K, | GGGGSGGGGSGGGGSGGGGS |
| D20A, M104V) | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 08) |
| | Comprising: |
| | MYRMQLLSCIALSLALVTNSAPTSSSTKKTQLQLEHLLLDLQMILNGINNYK |
| | NPKLTRMLTFKFYMPKKATELKHLQCLEEELKPLEEVLNLAQSKNFHLRPR |
| | DLISNINVIVLELKGSETTFMCEYADETATIVEFLNRWITFCQSIISTLT (WT |
| | IL-2, SEQ ID NO: 5) |
| | GGGGSGGGGSGGGGSGGGGS ((G₄S)₄ linker, SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 2) |
| | GGGGSGGGGSGGGGSGGGGS ((G₄S)₄ linker, SEQ ID NO: 6) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 4) |
| SEQ ID NO: 09 | MGLTSQLLPPLFFLLACAGNFVHGHKCDITLQEIIKTLNSLTEQKTLCTELTV |
| Bivalent bitargeted | TDIFAASKNTTEKETFCRAATVLRQFYSHHEKDTRCLGATAQQFHRHKQLI |
| IL-4 and signaling | RFLKRLDRNLWGLAGLNSCPVKEANQSTLENFLERLKTIMREKYSKCSS |
| deficient IL-2 | GGGGSGGGGSGGGGSGGGGS |
| Native IL-4 | DKTHTCPPCPAPELLGGPSVFLFPPKPKDTLMISRTPEVTCVVVDVSHEDPE |
| (G₄S)₄ | VKFNWYVDGVEVHNAKTKPREEQYNSTYRVVSVLTVLHQDWLNGKEYK |
| Monovalent Fc | CKVSNKALPAPIEKTISKAKGQPREPQVYTLPPSRDELTKNQVNLTCLVKGF |
| (G₄S)₄ | YPSDIAVEWESNGQPENNYKTTPPVLDSDGSFFLNSTLTVDKSRWQQGNVF |
| IL-2(21-153; V91K, | SCSVMHEALHNHYTQKSLSLSP |
| D20A, M104V) | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 09) |
| | Comprising: |
| | MGLTSQLLPPLFFLLACAGNFVHGHKCDITLQEIIKTLNSLTEQKTLCTELTV |
| | TDIFAASKNTTEKETFCRAATVLRQFYSHHEKDTRCLGATAQQFHRHKQLI |
| | RFLKRLDRNLWGLAGLNSCPVKEANQSTLENFLERLKTIMREKYSKCSS |
| | (WT IL-4, SEQ ID NO: 10) |
| | GGGGSGGGGSGGGGSGGGGS ((G₄S)₄ linker, SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 10 | MGLTSQLLPPLFFLLACAGNFVHGHKCDITLQEIIKTLNSLTEQKTLCTELTV |
| WT IL-4 (with | TDIFAASKNTTEKETFCRAATVLRQFYSHHEKDTRCLGATAQQFHRHKQLI |
| Signal Sequence) | RFLKRLDRNLWGLAGLNSCPVKEANQSTLENFLERLKTIMREKYSKCSS |
| | (WT IL-4, SEQ ID NO: 10) |
| SEQ ID NO: 11 | MFHVSFRYIFGLPPLILVLLPVASSDCDIEGKDGKQYESVLMVSIDQLLDSM |
| Bivalent bitargeted | KEIGSNCLNNEFNFFKRHICDANKEGMFLFRAARKLRQFLKMNSTGDFDLH |
| WT IL-7 and | LLKVSEGTTILLNCTGQVKGRKPAALGEAQPTKSLEENKSLKEQKKLNDLC |
| signaling deficient | FLKRLLQEIKTCWNKILMGTKEH |
| IL-2 | GGGGSGGGGSGGGGSGGGGS |
| Native IL-7 | DKTHTCPPCPAPELLGGPSVFLFPPKPKDTLMISRTPEVTCVVVDVSHEDPE |
| (G₄S)₄ | VKFNWYVDGVEVHNAKTKPREEQYNSTYRVVSVLTVLHQDWLNGKEYK |
| monovalent Fc | CKVSNKALPAPIEKTISKAKGQPREPQVYTLPPSRDELTKNQVNLTCLVKGF |
| (G₄S)₄ | YPSDIAVEWESNGQPENNYKTTPPVLDSDGSFFLNSTLTVDKSRWQQGNVF |
| IL-2(21-153; V91K, | SCSVMHEALHNHYTQKSLSLSP |
| D20A, M104V) | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 11) |
| | Comprising: |
| | MFHVSFRYIFGLPPLILVLLPVASSDCDIEGKDGKQYESVLMVSIDQLLDSM |
| | KEIGSNCLNNEFNFFKRHICDANKEGMFLFRAARKLRQFLKMNSTGDFDLH |
| | LLKVSEGTTILLNCTGQVKGRKPAALGEAQPTKSLEENKSLKEQKKLNDLC |
| | FLKRLLQEIKTCWNKILMGTKEH (WT IL-7, SEQ ID NO: 12) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 12 | MFHVSFRYIFGLPPLILVLLPVASSDCDIEGKDGKQYESVLMVSIDQLLDSM |
| WT IL-7 (with | KEIGSNCLNNEFNFFKRHICDANKEGMFLFRAARKLRQFLKMNSTGDFDLH |
| Signal Sequence | LLKVSEGTTILLNCTGQVKGRKPAALGEAQPTKSLEENKSLKEQKKLNDLC |
| | FLKRLLQEIKTCWNKILMGTKEH (WT IL-7, SEQ ID NO: 12) |
| SEQ ID NO: 13 | MLLAMVLTSALLLCSVAGQGCPTLAGILDINFLINKMQEDPASKCHCSANV |
| Bivalent bitargeted | TSCLCLGIPSDNCTRPCFSERLSQMTNTTMQTRYPLIFSRVKKSVEVLKNNK |
| WT IL-9 and | CPYFSCEQPCNQTTAGNALTFLKSLLEIFQKEKMRGMRGKI |
| signaling deficient | GGGGSGGGGSGGGGSGGGGS |
| IL-2 | DKTHTCPPCPAPELLGGPSVFLFPPKPKDTLMISRTPEVTCVVVDVSHEDPE |
| Native IL-9 | VKFNWYVDGVEVHNAKTKPREEQYNSTYRVVSVLTVLHQDWLNGKEYK |
| (G₄S)₄ | CKVSNKALPAPIEKTISKAKGQPREPQVYTLPPSRDELTKNQVNLTCLVKGF |
| Monovalent Fc | YPSDIAVEWESNGQPENNYKTTPPVLDSDGSFFLNSTLTVDKSRWQQGNVF |
| (G₄S)₄ | SCSVMHEALHNHYTQKSLSLSP |
| IL-2(21-153; V91K, | GGGGSGGGGSGGGGSGGGGS |
| D20A, M104V) | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 13) |
| | Comprising: |
| | MLLAMVLTSALLLCSVAGQGCPTLAGILDINFLINKMQEDPASKCHCSANV |
| | TSCLCLGIPSDNCTRPCFSERLSQMTNTTMQTRYPLIFSRVKKSVEVLKNNK |
| | CPYFSCEQPCNQTTAGNALTFLKSLLEIFQKEKMRGMRGKI (WT IL-9, SEQ |
| | ID NO: 14) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYR VVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 2) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 14 | MLLAMVLTSALLLCSVAGQGCPTLAGILDINFLINKMQEDPASKCHCSANV |
| WT IL-9 (with | TSCLCLGIPSDNCTRPCFSERLSQMTNTTMQTRYPLIFSRVKKSVEVLKNNK |
| Signal Sequence) | CPYFSCEQPCNQTTAGNALTFLKSLLEIFQKEKMRGMRGKI (WT IL-9, SEQ |
| | ID NO: 14) |
| SEQ ID NO: 15 | MRISKPHLRSISIQCYLCLLLNSHFLTEAGIHVFILGCFSAGLPKTEANWVNVI |
| Bivalent bitargeted | SDLKKIEDLIQSMHIDATLYTESDVHPSCKVTAMKCFLLELQVISLESGDASI |
| WT IL-15 and | HDTVENLIILANNSLSSNGNVTESGCKECEELEEKNIKEFLQSFVHIVQMFIN |
| signaling deficient | TS |
| IL-2 | GGGGSGGGGSGGGGSGGGGS |
| Native IL-15 | DKTHTCPPCPAPELLGGPSVFLFPPKPKDTLMISRTPEVTCVVVDVSHEDPE |
| (G₄S)₄ | VKFNWYVDGVEVHNAKTKPREEQYNSTYRVVSVLTVLHQDWLNGKEYK |
| Monovalent Fc | CKVSNKALPAPIEKTISKAKGQPREPQVYTLPPSRDELTKNQVNLTCLVKGF |
| (G₄S)₄ | YPSDIAVEWESNGQPENNYKTTPPVLDSDGSFFLNSTLTVDKSRWQQGNVF |
| IL-2(21-153; V91K, | SCSVMHEALHNHYTQKSLSLSP |
| D20A, M104V) | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 15) |
| | Comprising: |
| | MRISKPHLRSISIQCYLCLLLNSHFLTEAGIHVFILGCFSAGLPKTEANWVNVI |
| | SDLKKIEDLIQSMHIDATLYTESDVHPSCKVTAMKCFLLELQVISLESGDASI |
| | HDTVENLIILANNSLSSNGNVTESGCKECEELEEKNIKEFLQSFVHIVQMFIN |
| | TS (WT IL-15e, SEQ ID NO: 16) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 16 | MRISKPHLRSISIQCYLCLLLNSHFLTEAGIHVFILGCFSAGLPKTEANWVNVI |
| WT IL-15 (with | SDLKKIEDLIQSMHIDATLYTESDVHPSCKVTAMKCFLLELQVISLESGDASI |
| Signal Sequence) | HDTVENLIILANNSLSSNGNVTESGCKECEELEEKNIKEFLQSFVHIVQMFIN |
| | TS (WT IL-15, SEQ ID NO: 16) |
| SEQ ID NO: 17 | MRSSPGNMERIVICLMVIFLGTLVHKSSSQGQDRHMIRMRQLIDIVDQLKNY |
| Bivalent bitargeted | VNDLVPEFLPAPEDVETNCEWSAFSCFQKAQLKSANTGNNERIINVSIKKLK |
| WT IL-21 and | RKPPSTNAGRRQKHRLTCPSCDSYEKKPPKEFLERFKSLLQKMIHQHLSSRT |
| signaling deficient | HGSEDS |
| IL-2 | GGGGSGGGGSGGGGSGGGGS |
| Native IL-21 | DKTHTCPPCPAPELLGGPSVFLFPPKPKDTLMISRTPEVTCVVVDVSHEDPE |
| (G₄S)₄ | VKFNWYVDGVEVHNAKTKPREEQYNSTYRVVSVLTVLHQDWLNGKEYK |
| Monovalent Fc | CKVSNKALPAPIEKTISKAKGQPREPQVYTLPPSRDELTKNQVNLTCLVKGF |
| (G₄S)₄ | YPSDIAVEWESNGQPENNYKTTPPVLDSDGSFFLNSTLTVDKSRWQQGNVF |
| IL-2(21-153; V91K, | SCSVMHEALHNHYTQKSLSLSP |
| D20A, M104V) | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 17) |
| | Comprising: |
| | MRSSPGNMERIVICLMVIFLGTLVHKSSSQGQDRHMIRMRQLIDIVDQLKNY |
| | VNDLVPEFLPAPEDVETNCEWSAFSCFQKAQLKSANTGNNERIINVSIKKLK |
| | RKPPSTNAGRRQKHRLTCPSCDSYEKKPPKEFLERFKSLLQKMIHQHLSSRT |
| | HGSEDS (WT IL-21, SEQ ID NO: 18) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 18 | MRSSPGNMERIVICLMVIFLGTLVHKSSSQGQDRHMIRMRQLIDIVDQLKNY |
| WT IL-21 (with | VNDLVPEFLPAPEDVETNCEWSAFSCFQKAQLKSANTGNNERIINVSIKKLK |
| Signal Sequence) | RKPPSTNAGRRQKHRLTCPSCDSYEKKPPKEFLERFKSLLQKMIHQHLSSRT |
| | HGSEDS (WT IL-21, SEQ ID NO: 18) |
| SEQ ID NO: 19 | MYRMQLLSCIALSLALVTNSAPTSSSTKKTQLQLEHLLLDLQMILNGINNYK |
| Bivalent bitargeted | NPKLTRMLTFKFYMPKKATELKHLQCLEEELKPLEEVLNLAQSKNFHLRPR |
| WT IL-2 and | DLISNINVIVLELKGSETTFMCEYADETATIVEFLNRWITFCQSIISTLT |
| signaling deficient | GGGGSGGGGSGGGGSGGGGS |
| IL-2 | APTSSSTKKTQLQLEHLLLALQMILNGINNYKNPKLTRMLTFKFYMPKKAT |
| Native IL-2 | ELKHLQCLEEELKPLEEVLNLAQSKNFHLRPRDLISNINKIVLELKGSETTFV |
| (G₄S)₄ | CEYADETATIVEFLNRWITFCQSIISTLT (SEQ ID NO: 19) |
| IL-2(21-153; V91K, | Comprising: |
| D20A, M104V) | MYRMQLLSCIALSLALVTNSAPTSSSTKKTQLQLEHLLLDLQMILNGINNYK |
| | NPKLTRMLTFKFYMPKKATELKHLQCLEEELKPLEEVLNLAQSKNFHLRPR |
| | DLISNINVIVLELKGSETTFMCEYADETATIVEFLNRWITFCQSIISTLT (WT |
| | IL-2, SEQ ID NO: 05) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 20 | MGLTSQLLPPLFFLLACAGNFVHGHKCDITLQEIIKTLNSLTEQKTLCTELTV |
| Tetravalent | TDIFAASKNTTEKETFCRAATVLRQFYSHHEKDTRCLGATAQQFHRHKQLI |
| bitargeted | RFLKRLDRNLWGLAGLNSCPVKEANQSTLENFLERLKTIMREKYSKCSS |
| WT IL-4 and | GGGGSGGGGSGGGGSGGGGS |
| signaling deficient | DKTHTCPPCPAPELLGGPSVFLFPPKPKDTLMISRTPEVTCVVVDVSHEDPE |
| IL-2 | VKFNWYVDGVEVHNAKTKPREEQYNSTYRVVSVLTVLHQDWLNGKEYK |
| Native IL-4 | CKVSNKALPAPIEKTISKAKGQPREPQVYTLPPSRDELTKNQVSLTCLVKGF |
| (G₄S)₄ | YPSDIAVEWESNGQPENNYKTTPPVLDSDGSFFLYSKLTVDKSRWQQGNVF |
| Dimerizing Fc | SCSVMHEALHNHYTQKSLSLSP |
| (G₄S)₄ | GGGGSGGGGSGGGGSGGGGS |
| IL-4(25-153; | HKCDITLQEIIKTLNSLTEQKTLCTELTVTDIFAASKNTTEKETFCRAATVLR |
| R121D/Y124D) | QFYSHHEKDTRCLGATAQQFHRHKQLIRFLKRLDRNLWGLAGLNSCPVKE |
| | ANQSTLENFLERLKTIMDEKDSKCSS (SEQ ID NO: 20) |
| | Comprising: |
| | MGLTSQLLPPLFFLLACAGNFVHGHKCDITLQEIIKTLNSLTEQKTLCTELTV |
| | TDIFAASKNTTEKETFCRAATVLRQFYSHHEKDTRCLGATAQQFHRHKQLI |
| | RFLKRLDRNLWGLAGLNSCPVKEANQSTLENFLERLKTIMREKYSKCSS |
| | (WT IL-4, SEQ ID NO: 10) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 03) |
| | GGGGSGGGGSGGGGSGGGGS ((G₄S)₄ linker, SEQ ID NO: 06) |
| | HKCDITLQEIIKTLNSLTEQKTLCTELTVTDIFAASKNTTEKETFCRAATVLR |
| | QFYSHHEKDTRCLGATAQQFHRHKQLIRFLKRLDRNLWGLAGLNSCPVKE |
| | ANQSTLENFLERLKTIMDEKDSKCSS (Signaling deficient IL-4, SEQ ID |
| | NO: 21) |
| SEQ ID NO: 21 | HKCDITLQEIIKTLNSLTEQKTLCTELTVTDIFAASKNTTEKETFCRAATVLR |
| Signaling deficient | QFYSHHEKDTRCLGATAQQFHRHKQLIRFLKRLDRNLWGLAGLNSCPVKE |
| IL-4 (R121D and | ANQSTLENFLERLKTIMDEKDSKCSS (Signaling deficient IL-4, SEQ ID |
| Y124D | NO: 21) |
| SEQ ID NO: 22 | MPPSGLRLLLLLLPLLWLLVLTPGRPAAGLSTCKTIDMELVKRKRIEAIRGQI |
| Tetravalent | LSKLRLASPPSQGEVPPGPLPEAVLALYNSTRDRVAGESAEPEPEPEADYYA |
| bitargeted | KEVTRVLMVETHNEIYDKFKQSTHSIYMFFNTSELREAVPEPVLLSRAELRL |
| IL2Ra-targeted WT | LRLKLKVEQHVELYQKYSNNSWRYLSNRLLAPSDSPEWLSFDVTGVVRQW |
| TGFβ | LSRGGEIEGFRLSAHCSCDSRDNTLQVDINGFTTGRRGDLATIHGMNRPFLL |
| Native TGFβ | LMATPLERAQHLQSSRHRRALDTNYCFSSTEKNCCVRQLYIDFRKDLGWK |
| (G₄S)₄ | WIHEPKGYHANFCLGPCPYIWSLDTQYSKVLALYNQHNPGASAAPCCVPQ |
| Dimerizing Fc | ALEPLPIVYYVGRKPKVEQLSNMIVRSCKCS |
| (G₄S)₄ | GGGGSGGGGSGGGGSGGGGS |
| IL-2(21-153; V91K, | DKTHTCPPCPAPELLGGPSVFLFPPKPKDTLMISRTPEVTCVVVDVSHEDPE |
| D20A, M104V) | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP |
| | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 22) |
| | Comprising: |
| | MPPSGLRLLLLLLPLLWLLVLTPGRPAAGLSTCKTIDMELVKRKRIEAIRGQI |
| | LSKLRLASPPSQGEVPPGPLPEAVLALYNSTRDRVAGESAEPEPEPEADYYA |
| | KEVTRVLMVETHNEIYDKFKQSTHSIYMFFNTSELREAVPEPVLLSRAELRL |
| | LRLKLKVEQHVELYQKYSNNSWRYLSNRLLAPSDSPEWLSFDVTGVVRQW |
| | LSRGGEIEGFRLSAHCSCDSRDNTLQVDINGFTTGRRGDLATIHGMNRPFLL |
| | LMATPLERAQHLQSSRHRRALDTNYCFSSTEKNCCVRQLYIDFRKDLGWK |
| | WIHEPKGYHANFCLGPCPYIWSLDTQYSKVLALYNQHNPGASAAPCCVPQ |
| | ALEPLPIVYYVGRKPKVEQLSNMIVRSCKCS (TGFß, SEQ ID NO: 23) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 02) |
| | GGGGSGGGGSGGGGSGGGGS ((G₄S)₄ linker) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 23 | MPPSGLRLLLLLLPLLWLLVLTPGRPAAGLSTCKTIDMELVKRKRIEAIRGQI |
| TGFβ | LSKLRLASPPSQGEVPPGPLPEAVLALYNSTRDRVAGESAEPEPEPEADYYA |
| | KEVTRVLMVETHNEIYDKFKQSTHSIYMFFNTSELREAVPEPVLLSRAELRL |
| | LRLKLKVEQHVELYQKYSNNSWRYLSNRLLAPSDSPEWLSFDVTGVVRQW |
| | LSRGGEIEGFRLSAHCSCDSRDNTLQVDINGFTTGRRGDLATIHGMNRPFLL |
| | LMATPLERAQHLQSSRHRRALDTNYCFSSTEKNCCVRQLYIDFRKDLGWK |
| | WIHEPKGYHANFCLGPCPYIWSLDTQYSKVLALYNQHNPGASAAPCCVPQ |
| | ALEPLPIVYYVGRKPKVEQLSNMIVRSCKCS (TGFβ, SEQ ID NO: 23) |
| SEQ ID NO: 24 | MVPSAGQLALFALGIVLAACQALENSTSPLSADPPVAAAVVSHENDCPDSH |
| Tetravalent | TQFCFHGTCRFLVQEDKPACVCHSGYVGARCEHADLLAVVAASQKKQAIT |
| bitargeted | ALVVVSIVALAVLIITCVLIHCCQVRKHCEWCRALICRHEKPSALLKGRTAC |
| IL2Ra-targeted WT | CHSETVV |
| TGFα | GGGGSGGGGSGGGGSGGGGS |
| Native TGFα | DKTHTCPPCPAPELLGGPSVFLFPPKPKDTLMISRTPEVTCVVVDVSHEDPE |
| (G₄S)₄ | VKFNWYVDGVEVHNAKTKPREEQYNSTYRVVSVLTVLHQDWLNGKEYK |
| Dimerizing Fc | CKVSNKALPAPIEKTISKAKGQPREPQVYTLPPSRDELTKNQVSLTCLVKGF |
| (G₄S)₄ | YPSDIAVEWESNGQPENNYKTTPPVLDSDGSFFLYSKLTVDKSRWQQGNVF |
| IL-2(21-153; V91K, | SCSVMHEALHNHYTQKSLSLSP |
| D20A, M104V) | GGGGSGGGGSGGGGSGGGGS |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (SEQ ID NO: 24) |
| | Comprising: |
| | MVPSAGQLALFALGIVLAACQALENSTSPLSADPPVAAAVVSHFNDCPDSH |
| | TQFCFHGTCRFLVQEDKPACVCHSGYVGARCEHADLLAVVAASQKKQAIT |
| | ALVVVSIVALAVLIITCVLIHCCQVRKHCEWCRALICRHEKPSALLKGRTAC |
| | CHSETVV (TGFα. SEQ ID NO: 25) |
| | GGGGSGGGGSGGGGSGGGGS ((G₄S)₄ linker, SEQ ID NO: 06) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 03) |
| | GGGGSGGGGSGGGGSGGGGS ((G₄S)₄ linker, SEQ ID NO: 06) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 25 | MVPSAGQLALFALGIVLAACQALENSTSPLSADPPVAAAVVSHENDCPDSH |
| TGFα | TQFCFHGTCRFLVQEDKPACVCHSGYVGARCEHADLLAVVAASQKKQAIT |
| | ALVVVSIVALAVLIITCVLIHCCQVRKHCEWCRALICRHEKPSALLKGRTAC |
| | CHSETVV (TGFα. SEQ ID NO: 25) |
| SEQ ID NO: 26 | MGLTSQLLPPLFFLLACAGNFVHGHKCDITLQEIIKTLNSLTEQKTLCTELTV |
| Bivalent bitargeted | TDIFAASKNTTEKETFCRAATVLRQFYSHHEKDTRCLGATAQQFHRHKQLI |
| WT IL-4 and | RFLKRLDRNLWGLAGLNSCPVKEANQSTLENFLERLKTIMREKYSKCSS |
| signaling deficient | GGGGSGGGGSGGGGSGGGGS |
| IL-4 | HKCDITLQEIIKTLNSLTEQKTLCTELTVTDIFAASKNTTEKETFCRAATVLR |
| Native IL-4 | QFYSHHEKDTRCLGATAQQFHRHKQLIRFLKRLDRNLWGLAGLNSCPVKE |
| (G₄S)₄ | ANQSTLENFLERLKTIMDEKDSKCSS (SEQ ID NO: 26) |
| IL-4(25-153; | comprising |
| R121D/Y124D) | MGLTSQLLPPLFFLLACAGNFVHGHKCDITLQEIIKTLNSLTEQKTLCTELTV |
| | TDIFAASKNTTEKETFCRAATVLRQFYSHHEKDTRCLGATAQQFHRHKQLI |
| | RFLKRLDRNLWGLAGLNSCPVKEANQSTLENFLERLKTIMREKYSKCSS |
| | (WT IL-4 with Signaling Sequence, SEQ ID NO: 10) |
| | GGGGSGGGGSGGGGSGGGGS ((G₄S)₄ linker, SEQ ID NO: 06) |
| | HKCDITLQEIIKTLNSLTEQKTLCTELTVTDIFAASKNTTEKETFCRAATVLR |
| | QFYSHHEKDTRCLGATAQQFHRHKQLIRFLKRLDRNLWGLAGLNSCPVKE |
| | ANQSTLENFLERLKTIMDEKDSKCSS (Signaling deficient IL-4, SEQ ID |
| | NO: 21) |
| SEQ ID NO: 27 | APTSSSTKKTQLQLENLLLDLQMILNGINNYKNPKLTQMLTFKFYMPKKAT |
| IL-2 with R38Q and | ELKHLQCLEEELKPLEEVLNLAQSKNFHLRPRDLISNINVIVLELKGSETTFM |
| H16N, without | CEYADETATIVEFLNRWITFCQSIISTLT (SEQ ID NO: 27) |
| signal sequence | |
| SEQ ID NO: 28 | MYRMQLLSCIALSLALVTNSAPTSSSTKKTQLQLENLLLDLQMILNGINNYK |
| IL-2 (with signal | NPKLTQMLTFKFYMPKKATELKHLQCLEEELKPLEEVLNLAQSKNFHLRPR |
| sequence) with | DLISNINVIVLELKGSETTFMCEYADETATIVEFLNRWITFCQSIISTLT (SEQ |
| R38Q and H16N | ID NO: 28) |
| SEQ ID NO: 29 | MYRMQLLSCIALSLALVTNSAPTSSSTKKTQLQLENLLLDLQMILNGINNYK |
| Tetravalent | NPKLTQMLTFKFYMPKKATELKHLQCLEEELKPLEEVLNLAQSKNFHLRPR |
| IL-2 | DLISNINVIVLELKGSETTFMCEYADETATIVEFLNRWITFCQSIISTLTGGGG |
| (R38Q/H16N) and | SGGGGSGGGGSGGGGSDKTHTCPPCPAPELLGGPSVFLFPPKPKDTLMISRT |
| IL-2 | PEVTCVVVDVSHEDPEVKFNWYVDGVEVHNAKTKPREEQYNSTYRVVSV |
| (R38Q/H16N) | LTVLHQDWLNGKEYKCKVSNKALPAPIEKTISKAKGQPREPQVYTLPPSRD |
| IL-2 (R38Q, | ELTKNQVSLTCLVKGFYPSDIAVEWESNGQPENNYKTTPPVLDSDGSFFLYS |
| H16N) | KLTVDKSRWQQGNVFSCSVMHEALHNHYTQKSLSLSPGGGGGSGGGGSG |
| (G₄S)₄ | GGGSGGGGSAPTSSSTKKTQLQLENLLLDLQMILNGINNYKNPKLTQMLTF |
| Dimerizing Fc | KFYMPKKATELKHLQCLEEELKPLEEVLNLAQSKNFHLRPRDLISNINVIVL |
| (G₄S)₄ | ELKGSETTFMCEYADETATIVEFLNRWITFCQSIISTLT (SEQ ID NO: 29) |
| IL-2 (21-153; | Comprising: |
| R38Q, H16N) | MYRMQLLSCIALSLALVTNSAPTSSSTKKTQLQLENLLLDLQMILNGINNYK |
| | NPKLTQMLTFKFYMPKKATELKHLQCLEEELKPLEEVLNLAQSKNFHLRPR |
| | DLISNINVIVLELKGSETTFMCEYADETATIVEFLNRWITFCQSIISTLT (IL-2 |
| | R38Q, H16N, SEQ ID NO: 28) |
| | GGGGSGGGGSGGGGSGGGGS (SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 3) |
| | GGGGGSGGGGSGGGGSGGGGS (SEQ ID NO: 6) |
| | APTSSSTKKTQLQLENLLLDLQMILNGINNYKNPKLTQMLTFKFYMPKKAT |
| | ELKHLQCLEEELKPLEEVLNLAQSKNFHLRPRDLISNINVIVLELKGSETTFM |
| | CEYADETATIVEFLNRWITFCQSIISTLT (IL-2 [21-153], R38Q, H16N, SEQ ID |
| | NO: 27) |
| SEQ ID NO: 30 | ATGTATAGAATGCAACTGCTCTCCTGCATAGCATTAAGCCTCGCCTTAGTCACTAATAGCGCTCCGA |
| Encodes SEQ ID | CATCAAGTAGTACGAAGAAAACACAACTGCAACTTGAGAATCTTCTACTGGATCTCCAGATGATCCT |
| NO: 29 | TAACGGTATCAACAACTATAAGAATCCCAAGTTAACACAAATGTTGACCTTTAAATTCTACATGCCC |
| | AAGAAGGCCACAGAATTGAAGCATCTCCAATGTCTTGAGGAAGAGCTGAAACCCCTTGAGGAAGTGT |
| | TAAATCTGGCACAGTCTAAGAACTTTCATCTCCGCCCCAGAGATTTAATATCTAATATCAATGTGAT |
| | CGTTCTGGAACTGAAGGGTTCAGAAACCACTTTCATGTGCGAGTATGCTGATGAGACAGCCACAATT |
| | GTAGAATTCCTCAACAGGTGGATAACATTCTGCCAGTCCATCATTAGTACATTAACTGGTGGCGGTG |
| | GATCAGGGGGAGGCGGATCAGGTGGCGGAGGATCCGGTGGAGGAGGTTCCGATAAGACCCATACTT |
| | GTCCACCGTGTCCAGCTCCAGAATTGTTGGGTGGACCTAGCGTGTTTCTGTTCCCACCCAAGCCCA |
| | AAGATACACTTATGATCAGTCGGACTCCTGAGGTAACGTGCGTGGTAGTGGACGTCAGCCATGAGG |
| | ACCCAGAGGTCAAATTTAACTGGTATGTTGATGGAGTTGAAGTGCATAACGCCAAAACTAAACCAAG |
| | GGAAGAACAGTACAACTCTACCTATAGAGTGGTTTCCGTACTCACAGTACTGCACCAAGACTGGCTG |
| | AACGGAAAAGAATATAAATGTAAGGTGAGCAATAAAGCATTGCCCGCCCCGATCGAGAAAACAATTT |
| | CAAAAGCTAAAGGCCAACCGAGAGAACCCCAAGTTTATACCCTTCCACCCTCTCGGGATGAGTTGA |
| | CCAAGAACCAAGTGTCATTAACTTGTCTGGTGAAGGGATTTTATCCCTCTGACATTGCCGTGGAATG |
| | GGAATCCAACGGTCAGCCGGAAAACAATTATAAAACTACTCCACCCGTCTTAGACTCTGACGGGAG |
| | TTTCTTCCTGTACAGCAAGCTTACAGTAGACAAGTCTCGGTGGCAACAAGGAAACGTCTTCAGCTGT |
| | AGCGTCATGCATGAAGCATTACATAATCACTATACACAAAAGTCTCTGTCACTGTCCCCTGGCGGTG |
| | GAGGAGGCAGCGGCGGTGGTGGTTCTGGAGGAGGTGGAAGTGGTGGCGGCGGGAGTGCACCAA |
| | CTAGCAGTTCCACCAAAAAGACTCAGTTACAGCTCGAAAACCTGCTGCTTGACCTGCAGATGATTCT |
| | GAATGGAATAAATAATTACAAAAACCCTAAGCTCACACAGATGCTTACGTTCAAGTTTTATATGCCA |
| | AAGAAGGCTACGGAGCTCAAACACCTGCAATGCTTGGAAGAGGAACTCAAGCCATTGGAAGAGGTTC |
| | TGAACCTGGCTCAATCCAAAAATTTCCACCTTAGGCCTAGAGACCTCATCAGCAACATAAACGTTAT |
| | AGTACTAGAGCTCAAAGGGAGCGAGACGACCTTTATGTGCGAGTACGCCGATGAAACTGCTACCAT |
| | CGTGGAGTTTTTGAATAGATGGATCACCTTTTGTCAAAGCATTATATCCACACTTACCTGA (SEQ |
| | ID NO: 30) |
| SEQ ID NO: 31 | MYRMQLLSCIALSLALVTNSAPTSSSTKKTQLQLENLLLDLQMILNGINNYK |
| Bivalent | NPKLTQMLTFKFYMPKKATELKHLQCLEEELKPLEEVLNLAQSKNFHLRPR |
| IL-2 | DLISNINVIVLELKGSETTFMCEYADETATIVEFLNRWITFCQSIISTLTGGGG |
| (R38Q/H16N) and | SGGGGSGGGGSGGGGSDKTHTCPPCPAPELLGGPSVFLFPPKPKDTLMISRT |
| Dimerizing Fc | PEVTCVVVDVSHEDPEVKFNWYVDGVEVHNAKTKPREEQYNSTYRVVSV |
| IL-2 (R38Q, | LTVLHQDWLNGKEYKCKVSNKALPAPIEKTISKAKGQPREPQVYTLPPSRD |
| H16N), | ELTKNQVSLTCLVKGFYPSDIAVEWESNGQPENNYKTTPPVLDSDGSFFLYS |
| (G₄S)₄ | KLTVDKSRWQQGNVFSCSVMHEALHNHYTQKSLSLSP (SEQ ID NO: 31) |
| Dimerizing Fc | Comprising: |
| | MYRMQLLSCIALSLALVTNSAPTSSSTKKTQLQLENLLLDLQMILNGINNYK |
| | NPKLTQMLTFKFYMPKKATELKHLQCLEEELKPLEEVLNLAQSKNFHLRPR |
| | DLISNINVIVLELKGSETTFMCEYADETATIVEFLNRWITFCQSIISTLT (IL-2 |
| | R38Q, H16N, SEQ ID NO: 28) |
| | GGGGSGGGGSGGGGSGGGGS (SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 03) |
| SEQ ID NO: 32 | ATGTATCGCATGCAACTCTTGTCTTGTATTGCCCTCTCTCTCGCCCTCGTCACGAACTCTGCTCCAA |
| Encodes SEQ ID | CAAGTAGTTCAACCAAGAAAACCCAATTACAATTGGAAAACTTGCTGCTGGACTTACAGATGATACT |
| NO: 31 | TAACGGCATCAATAACTACAAGAACCCAAAGCTCACACAAATGCTTACATTTAAATTCTATATGCCG |
| | AAGAAAGCAACAGAGCTCAAGCATCTCCAATGCCTCGAAGAAGAGTTAAAGCCTCTCGAGGAAGTTC |
| | TCAATCTTGCCCAATCCAAGAACTTTCATCTTAGACCGCGAGATCTGATATCTAATATAAATGTGAT |
| | TGTCCTTGAACTCAAAGGGTCAGAGACTACTTTCATGTGTGAGTATGCCGATGAGACGGCTACCATC |
| | GTGGAGTTCCTCAATAGGTGGATCACGTTTTGCCAAAGCATAATTTCCACCTTGACCGGCGGCGGTG |
| | GATCCGGTGGAGGAGGGAGTGGTGGCGGCGGAAGCGGCGGTGGTGGCTCCGATAAAACACACACA |
| | TGTCCCCCTTGTCCTGCACCAGAACTCCTGGGCGGCCCTTCAGTGTTCCTGTTTCCCCCAAAACCC |
| | AAAGACACATTGATGATCTCCCGCACTCCTGAGGTGACGTGTGTTGTCGTGGATGTTTCCCATGAAG |
| | ACCCGGAAGTTAAATTTAACTGGTACGTGGATGGCGTAGAAGTCCATAATGCCAAGACAAAGCCAC |
| | GGGAAGAGCAGTACAACTCTACCTATCGAGTGGTCTCAGTGTTGACCGTTCTGCACCAAGATTGGC |
| | TGAATGGGAAGGAGTATAAGTGTAAGGTTTCAAACAAAGCTCTGCCGGCACCCATTGAAAAGACAAT |
| | CTCTAAGGCAAAAGGCCAACCAAGAGAGCCACAAGTCTATACGCTGCCCCCTAGCCGGGATGAGCT |
| | CACTAAGAACCAGGTATCTCTCACATGTCTCGTCAAAGGGTTCTATCCATCTGATATCGCAGTGGAA |
| | TGGGAATCAAATGGACAGCCAGAGAACAATTATAAAACCACTCCACCCGTCCTTGATTCCGACGGG |
| | TCCTTCTTCTTATATTCCAAGTTAACTGTGGATAAATCTCGTTGGCAGCAAGGCAATGTCTTTAGTT |
| | GCTCCGTAATGCATGAAGCGCTCCATAACCATTACACACAGAAGTCACTGTCACTCAGCCCTTAA |
| | (SEQ ID NO: 32) |
| SEQ ID NO: 33 | MYRMQLLSCIALSLALVTNSAPTSSSTKKTQLQLENLLLDLQMILNGINNYK |
| Monovalent | NPKLTQMLTFKFYMPKKATELKHLQCLEEELKPLEEVLNLAQSKNFHLRPR |
| IL-2 (R38Q/H16) | DLISNINVIVLELKGSETTFMCEYADETATIVEFLNRWITFCQSIISTLTGGGG |
| IL-2 (R38Q, | SGGGGSGGGGSGGGGSDKTHTCPPCPAPELLGGPSVFLFPPKPKDTLMISRT |
| H16N) | PEVTCVVVDVSHEDPEVKFNWYVDGVEVHNAKTKPREEQYNSTYRVVSV |
| (G₄S)₄ | LTVLHQDWLNGKEYKCKVSNKALPAPIEKTISKAKGQPREPQVYTLPPSRD |
| Monovalent Fc | ELTKNQVNLTCLVKGFYPSDIAVEWESNGQPENNYKTTPPVLDSDGSFFLN |
| | STLTVDKSRWQQGNVFSCSVMHEALHNHYTQKSLSLSP (SEQ ID NO: 33) |
| | Comprising: |
| | MYRMQLLSCIALSLALVTNSAPTSSSTKKTQLQLENLLLDLQMILNGINNYK |
| | NPKLTQMLTFKFYMPKKATELKHLQCLEEELKPLEEVLNLAQSKNFHLRPR |
| | DLISNINVIVLELKGSETTFMCEYADETATIVEFLNRWITFCQSIISTLT (IL-2, |
| | R38Q, H16N, SEQ ID NO 28) |
| | GGGGSGGGGSGGGGSGGGGS (SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| SEQ ID NO: 34 | ATGTACCGAATGCAATTGCTTAGCTGTATTGCCTTGAGTCTCGCACTCGTGACTAACTCAGCCCCCA |
| Encodes SEQ ID | CCTCATCCAGCACTAAGAAAACGCAGCTGCAACTCGAGAACTTACTGCTTGATCTCCAGATGATTCT |
| NO: 33 | CAACGGCATCAACAATTACAAGAATCCAAAGTTGACACAAATGCTGACCTTTAAATTTTATATGCCA |
| | AAGAAGGCCACCGAGCTGAAGCATTTGCAGTGCCTTGAAGAGGAACTTAAACCACTGGAAGAAGTTT |
| | TGAACCTGGCCCAAAGCAAGAACTTCCACTTGAGACCTCGGGATCTGATAAGCAACATTAACGTTAT |
| | TGTCCTGGAGCTCAAAGGATCAGAAACAACCTTCATGTGTGAATACGCAGATGAGACTGCAACTATT |
| | GTAGAGTTTCTGAACCGCTGGATTACCTTCTGCCAGAGCATTATTTCCACACTCACAGGCGGAGGA |
| | GGATCAGGCGGCGGCGGTAGCGGCGGAGGAGGAAGCGGAGGAGGCGGCAGTGATAAGACACATA |
| | CATGTCCACCTTGTCCAGCCCCAGAGCTTTTGGGCGGCCCGTCCGTGTTTCTGTTTCCACCTAAGC |
| | CTAAAGATACATTAATGATCAGCCGGACTCCTGAAGTTACTTGTGTTGTGGTTGATGTCAGTCATGA |
| | GGATCCAGAGGTTAAGTTCAACTGGTACGTGGATGGTGTGGAAGTTCACAATGCGAAAACGAAACC |
| | ACGCGAAGAACAGTATAATAGTACATACCGTGTAGTGAGCGTATTGACTGTGCTCCATCAAGACTGG |
| | CTTAATGGCAAAGAATATAAGTGTAAGGTGAGTAACAAAGCGCTCCCCGCACCTATTGAGAAGACGA |
| | TTAGCAAAGCCAAGGGTCAACCACGCGAACCCCAAGTCTATACACTGCCCCCTTCCCGTGATGAGT |
| | TAACCAAGAATCAAGTCAATCTTACCTGTCTTGTAAAAGGTTTCTACCCGTCTGATATCGCCGTCGA |
| | ATGGGAGTCTAATGGCCAACCGGAAAACAACTACAAGACCACACCGCCCGTCCTCGACTCAGATGGG |
| | AGTTTCTTCCTGAATAGCACTTTGACAGTGGATAAAAGTCGATGGCAACAGGGGAATGTGTTCTCCT |
| | GTAGCGTAATGCATGAGGCTCTGCATAACCATTACACACAGAAATCACTCAGTCTCAGCCCTTAA |
| | (SEQ ID NO: 34) |
| SEQ ID NO: 35 | MYRMQLLSCIALSLALVTNSAPTSSSTKKTQLQLENLLLDLQMILNGINNYK |
| Tetravalent | NPKLTQMLTFKFYMPKKATELKHLQCLEEELKPLEEVLNLAQSKNFHLRPR |
| bitargeted | DLISNINVIVLELKGSETTFMCEYADETATIVEFLNRWITFCQSIISTLTGGGG |
| IL-2 (R38Q, | SGGGGSGGGGSGGGGSDKTHTCPPCPAPELLGGPSVFLFPPKPKDTLMISRT |
| H16N) and | PEVTCVVVDVSHEDPEVKFNWYVDGVEVHNAKTKPREEQYNSTYRVVSV |
| signaling deficient | LTVLHQDWLNGKEYKCKVSNKALPAPIEKTISKAKGQPREPQVYTLPPSRD |
| IL-2 | ELTKNQVSLTCLVKGFYPSDIAVEWESNGQPENNYKTTPPVLDSDGSFFLYS |
| IL-2 (R38Q, | KLTVDKSRWQQGNVFSCSVMHEALHNHYTQKSLSLSPGGGGSGGGGSGG |
| H16N) | GGSGGGGSAPTSSSTKKTQLQLEHLLLALQMILNGINNYKNPKLTRMLTFK |
| (G₄S)₄ | FYMPKKATELKHLQCLEEELKPLEEVLNLAQSKNFHLRPRDLISNINKIVLE |
| Dimerizing Fc | LKGSETTFVCEYADETATIVEFLNRWITFCQSIISTLT (SEQ ID NO: 35) |
| (G₄S)₄ | Comprising: |
| IL-2(21-153; | MYRMQLLSCIALSLALVTNSAPTSSSTKKTQLQLENLLLDLQMILNGINNYK |
| V91K, D20A, | NPKLTQMLTFKFYMPKKATELKHLQCLEEELKPLEEVLNLAQSKNFHLRPR |
| M104V) | DLISNINVIVLELKGSETTFMCEYADETATIVEFLNRWITFCQSIISTLT (II-2, |
| | R38Q, H16N, SEQ ID NO: 28) |
| | GGGGSGGGGSGGGGSGGGGS (SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVF |
| | SCSVMHEALHNHYTQKSLSLSP (Dimerizing Fc fragment, SEQ ID NO: 03) |
| | GGGGSGGGGSGGGGSGGGGS (SEQ ID NO: 6) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 36 | ATGTATAGAATGCAACTGCTCTCCTGCATAGCATTAAGCCTCGCCTTAGTCACTAATAGCGCTCCGA |
| Encodes SEQ ID | CATCAAGTAGTACGAAGAAAACACAACTGCAACTTGAGAATCTTCTACTGGATCTCCAGATGATCCT |
| NO: 35 | TAACGGTATCAACAACTATAAGAATCCCAAGTTAACACAAATGTTGACCTTTAAATTCTACATGCCCA |
| | AGAAGGCCACAGAATTGAAGCATCTCCAATGTCTTGAGGAAGAGCTGAAACCCCTTGAGGAAGTGT |
| | TAAATCTGGCACAGTCTAAGAACTTTCATCTCCGCCCCAGAGATTTAATATCTAATATCAATGTGATC |
| | GTTCTGGAACTGAAGGGTTCAGAAACCACTTTCATGTGCGAGTATGCTGATGAGACAGCCACAATTG |
| | TAGAATTCCTCAACAGGTGGATAACATTCTGCCAGTCCATCATTAGTACATTAACTGGTGGCGGTGG |
| | ATCAGGGGGAGGCGGATCAGGTGGCGGAGGATCCGGTGGAGGAGGTTCCGATAAGACCCATACTT |
| | GTCCACCGTGTCCAGCTCCAGAATTGTTGGGTGGACCTAGCGTGTTTCTGTTCCCACCCAAGCCCA |
| | AAGATACACTTATGATCAGTCGGACTCCTGAGGTAACGTGCGTGGTAGTGGACGTCAGCCATGAGG |
| | ACCCAGAGGTCAAATTTAACTGGTATGTTGATGGAGTTGAAGTGCATAACGCCAAAACTAAACCAAG |
| | GGAAGAACAGTACAACTCTACCTATAGAGTGGTTTCCGTACTCACAGTACTGCACCAAGACTGGCTG |
| | AACGGAAAAGAATATAAATGTAAGGTGAGCAATAAAGCATTGCCCGCCCCGATCGAGAAAACAATTT |
| | CAAAAGCTAAAGGCCAACCGAGAGAACCCCAAGTTTATACCCTTCCACCCTCTCGGGATGAGTTGA |
| | CCAAGAACCAAGTGTCATTAACTTGTCTGGTGAAGGGATTTTATCCCTCTGACATTGCCGTGGAATG |
| | GGAATCCAACGGTCAGCCGGAAAACAATTATAAAACTACTCCACCCGTCTTAGACTCTGACGGGAG |
| | TTTCTTCCTGTACAGCAAGCTTACAGTAGACAAGTCTCGGTGGCAACAAGGAAACGTCTTCAGCTGT |
| | AGCGTCATGCATGAAGCATTACATAATCACTATACACAAAAGTCTCTGTCACTGTCCCCTGGTGGAG |
| | GAGGCAGCGGCGGTGGTGGTTCTGGAGGAGGTGGAAGTGGTGGCGGCGGGAGTGCACCAACTAG |
| | CAGTTCCACCAAAAAGACTCAGTTACAGCTCGAACATCTGCTGCTTGCACTGCAGATGATTCTGAAT |
| | GGAATAAATAATTACAAAAACCCTAAGCTCACACGTATGCTTACGTTCAAGTTTTATATGCCAAAGAA |
| | GGCTACGGAGCTCAAACACCTGCAATGCTTGGAAGAGGAACTCAAGCCATTGGAAGAGGTTCTGAA |
| | CCTGGCTCAATCCAAAAATTTCCACCTTAGGCCTAGAGACCTCATCAGCAACATAAACAAGATAGTA |
| | CTAGAGCTCAAAGGGAGCGAGACGACCTTTGTTTGCGAGTACGCCGATGAAACTGCTACCATCGTG |
| | GAGTTTTTGAATAGATGGATCACCTTTTGTCAAAGCATTATATCCACACTTACCTGA (SEQ ID |
| | NO: 36) |
| SEQ ID NO: 37 | MYRMQLLSCIALSLALVTNSAPTSSSTKKTQLQLENLLLDLQMILNGINNYK |
| Bivalent bitargeted | NPKLTQMLTFKFYMPKKATELKHLQCLEEELKPLEEVLNLAQSKNFHLRPR |
| IL-2 R38Q, H16N) | DLISNINVIVLELKGSETTFMCEYADETATIVEFLNRWITFCQSIISTLTGGGG |
| and signaling | SGGGGSGGGGSGGGGSDKTHTCPPCPAPELLGGPSVFLFPPKPKDTLMISRT |
| deficient IL-2 | PEVTCVVVDVSHEDPEVKFNWYVDGVEVHNAKTKPREEQYNSTYRVVSV |
| IL-2 (R38Q, | LTVLHQDWLNGKEYKCKVSNKALPAPIEKTISKAKGQPREPQVYTLPPSRD |
| H16N) | ELTKNQVNLTCLVKGFYPSDIAVEWESNGQPENNYKTTPPVLDSDGSFFLN |
| (G₄S)₄ | STLTVDKSRWQQGNVFSCSVMHEALHNHYTQKSLSLSPGGGGSGGGGSGG |
| Monovalent Fc | GGSGGGGSAPTSSSTKKTQLQLEHLLLALQMILNGINNYKNPKLTRMLTFK |
| (G₄S)₄ | FYMPKKATELKHLQCLEEELKPLEEVLNLAQSKNFHLRPRDLISNINKIVLE |
| IL-2(21-153; | LKGSETTFVCEYADETATIVEFLNRWITFCQSIISTLT (SEQ ID NO: 37) |
| V91K, D20A, | Comprising: |
| M104V) | MYRMQLLSCIALSLALVTNSAPTSSSTKKTQLQLENLLLDLQMILNGINNYK |
| | NPKLTQMLTFKFYMPKKATELKHLQCLEEELKPLEEVLNLAQSKNFHLRPR |
| | DLISNINVIVLELKGSETTFMCEYADETATIVEFLNRWITFCQSIISTLT (IL-2, |
| | R38Q, H16N, SEQ ID NO: 28) |
| | GGGGSGGGGSGGGGSGGGGS (SEQ ID NO: 6) |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSRDELTKNQVNLTCLVKGF |
| | YPSDIAVEWESNGQPENNYKTTPPVLDSDGSFFLNSTLTVDKSRWQQGNVF |
| | SCSVMHEALHNHYTQKSLSLSP (Monovalent Fc fragment, SEQ ID NO: 02) |
| | GGGGSGGGGSGGGGSGGGGS (SEQ ID NO: 6) |
| | APTSSSTKKTQLQLEHLLLALQMILNGINNYKNPKLTRMLTFKFYMPKKAT |
| | ELKHLQCLEEELKPLEEVLNLAQSKNFHLRPRDLISNINKIVLELKGSETTFV |
| | CEYADETATIVEFLNRWITFCQSIISTLT (Signaling deficient IL-2, SEQ ID |
| | NO: 04) |
| SEQ ID NO: 38 | ATGTATAGAATGCAACTGCTCTCCTGCATAGCATTAAGCCTCGCCTTAGTCACTAATAGCGCTCCGA |
| Encodes SEQ ID | CATCAAGTAGTACGAAGAAAACACAACTGCAACTTGAGAATCTTCTACTGGATCTCCAGATGATCCT |
| NO: 37 | TAACGGTATCAACAACTATAAGAATCCCAAGTTAACACAAATGTTGACCTTTAAATTCTACATGCCC |
| | AAGAAGGCCACAGAATTGAAGCATCTCCAATGTCTTGAGGAAGAGCTGAAACCCCTTGAGGAAGTGT |
| | TAAATCTGGCACAGTCTAAGAACTTTCATCTCCGCCCCAGAGATTTAATATCTAATATCAATGTGAT |
| | CGTTCTGGAACTGAAGGGTTCAGAAACCACTTTCATGTGCGAGTATGCTGATGAGACAGCCACAATT |
| | GTAGAATTCCTCAACAGGTGGATAACATTCTGCCAGTCCATCATTAGTACATTAACTGGTGGCGGTG |
| | GATCAGGGGGAGGCGGATCAGGTGGCGGAGGATCCGGTGGAGGAGGTTCCGATAAGACCCATACTT |
| | GTCCACCGTGTCCAGCTCCAGAATTGTTGGGTGGACCTAGCGTGTTTCTGTTCCCACCCAAGCCCA |
| | AAGATACACTTATGATCAGTCGGACTCCTGAGGTAACGTGCGTGGTAGTGGACGTCAGCCATGAGG |
| | ACCCAGAGGTCAAATTTAACTGGTATGTTGATGGAGTTGAAGTGCATAACGCCAAAACTAAACCAAG |
| | GGAAGAACAGTACAACTCTACCTATAGAGTGGTTTCCGTACTCACAGTACTGCACCAAGACTGGCTG |
| | AACGGAAAAGAATATAAATGTAAGGTGAGCAATAAAGCATTGCCCGCCCCGATCGAGAAAACAATTT |
| | CAAAAGCTAAAGGCCAACCGAGAGAACCCCAAGTTTATACCCTTCCACCCTCTCGGGATGAGTTGA |
| | CCAAGAACCAAGTGAATTTAACTTGTCTGGTGAAGGGATTTTATCCCTCTGACATTGCCGTGGAATG |
| | GGAATCCAACGGTCAGCCGGAAAACAATTATAAAACTACTCCACCCGTCTTAGACTCTGACGGGAG |
| | TTTCTTCCTGAATAGCACCCTTACAGTAGACAAGTCTCGGTGGCAACAAGGAAACGTCTTCAGCTGT |
| | AGCGTCATGCATGAAGCATTACATAATCACTATACACAAAAGTCTCTGTCACTGTCCCCTGGTGGAG |
| | GAGGCAGCGGCGGTGGTGGTTCTGGAGGAGGTGGAAGTGGTGGCGGCGGGAGTGCACCAACTAG |
| | CAGTTCCACCAAAAAGACTCAGTTACAGCTCGAACATCTGCTGCTTGCACTGCAGATGATTCTGAAT |
| | GGAATAAATAATTACAAAAACCCTAAGCTCACACGTATGCTTACGTTCAAGTTTTATATGCCAAAGA |
| | AGGCTACGGAGCTCAAACACCTGCAATGCTTGGAAGAGGAACTCAAGCCATTGGAAGAGGTTCTGAA |
| | CCTGGCTCAATCCAAAAATTTCCACCTTAGGCCTAGAGACCTCATCAGCAACATAAACAAGATAGTA |
| | CTAGAGCTCAAAGGGAGCGAGACGACCTTTGTTTGCGAGTACGCCGATGAAACTGCTACCATCGTG |
| | GAGTTTTTGAATAGATGGATCACCTTTTGTCAAAGCATTATATCCACACTTACCTGA (SEQ ID |
| | NO: 38) |

### Cross-Linked Multispecific Multivalent Biomolecules

In some embodiments, a signaling-competent cytokine or variant thereof and one signaling-deficient cytokine or variant thereof are cross-linked to provide a multispecific multivalent biomolecule embodied herein. Various means of cross-linking are provided, and the disclosure is not so limited to any particular one. In one embodiment, the cross-linkable single-chain polypeptides cross-link upon expression.

As noted above, in one embodiment, two same or different single chain polypeptides comprising a dimerizable Fc region as described above may be expressed and cross-linked to form a dimer of single polypeptide chains. Such dimer can, in one embodiment, cross-link a signaling-competent cytokine or variant thereof to a signaling-deficient cytokine forming the multispecific multivalent biomolecule disclosed herein. In another embodiment, a cross-linkable single-chain polypeptide already comprising a signaling-competent cytokine or variant thereof and one signaling-deficient cytokine or variant thereof, can be cross-linked to another one or more cytokine to provide additional cytokines in the multispecific multivalent biomolecule disclosed here. Examples of each of these types of constructs are described below and are not intended to be limiting.

Such biomolecules may be prepared by cross-linking, for example, by co-expression, of a single-chain polypeptide comprising a single signaling-competent cytokine or variant and a dimerizable Fc region, with a single-chain polypeptide comprising a single signaling-deficient cytokine or variant thereof and a dimerizable Fc region.

As noted herein, in one embodiment, the multispecific multivalent biomolecule disclosed herein or one or more components thereof may be expressed as Fc fusion proteins with a human IgG Fc, such as a Fc from IgG1, IgG2, IgG3 or IgG4. In one embodiment, the native or altered common γ-chain receptor cytokine or immunologically active or altered fragment thereof is fused to the N terminus of human IgG1 Fc. In another embodiment, the native or altered common γ-chain receptor cytokine or immunologically active or altered fragment thereof is fused to the C terminus of human IgG1 Fc. One of ordinary skill in the art would readily use generally known techniques to construct the Fc fusion proteins disclosed herein. In one embodiment, the native or altered common γ-chain receptor cytokine or immunologically active or altered fragment thereof is fused to the N or C terminus of human IgG1 Fc through a linker. Linkers useful for making the Fc fusion proteins include, but are not limited to, (G₄S)₄ and other generally known linkers.

### Multispecific Multivalent Biomolecule Constructs: Cross-Linked Single Chain Polypeptides

As described herein, a multispecific multivalent biomolecule comprises at least one signaling-competent cytokine or variant thereof and at least one signaling-deficient cytokine or variant thereof which are covalently linked, and which the cytokines may be the same or different. As noted herein, in some embodiments, a multispecific multivalent biomolecule may be formed from the disulfide cross-linking of two single-chain polypeptides via Fc portions in each polypeptide. As further noted, while a single-chain polypeptide may not comprise both a native and altered cytokine, the cross-linked product comprises at least one of each, which may be the same or different cytokine.

Thus, in some embodiments, a multispecific multivalent biomolecule may comprise or consist of any two from among SEQ ID NOs: 07, 20, 22, 24, 29, 31 or 35. For example, two SEQ ID NO:07 form disulfide cross-links during expression, forming a multispecific multivalent biomolecule with two signaling-competent IL-2s and two signaling-deficient IL-2s. In another example, two SEQ ID NO:29 form disulfide cross-links during expression, forming a multispecific multivalent biomolecule with four signaling-competent IL-2s. In another example, two SEQ ID NO:31 form disulfide cross-links during expression, forming a multivalent biomolecule with two signaling-competent IL-2s. In another example, two SEQ ID NO: 35 form disulfide cross-links during expression, forming a multispecific multivalent biomolecule with two signaling-competent IL-2s and two signaling-deficient IL-2s. In another example, two SEQ ID NO:20 form disulfide cross-links during expression, forming a multispecific multivalent biomolecule with two signaling-competent IL-4s and two signaling-deficient IL-2s. In another example, two SEQ ID NO:22 form disulfide cross-links during expression, forming a multispecific multivalent biomolecule with two signaling-competent TGF-β and two signaling-deficient IL-2s. In another example, two SEQ ID NO:24 form disulfide cross-links during expression, forming a multispecific multivalent biomolecule with two signaling-competent TGF-α and two signaling-deficient IL-2s. In a similar fashion, any single-chain polypeptide such as those disclosed herein may be prepared using a dimerizable Fc region (such as in place of the monovalent Fc in SEQ ID NOs: 11, 13, 15, 17, 33, 37) to form a cross-linked multispecific multivalent biomolecule with two signaling-competent common γ-chain receptor cytokines and two signaling-deficient IL-2s. In some embodiments, cross-linking of different single-chain polypeptides (heterodimeric) is provided, such as a SEQ ID NO: 07 and a SEQ ID NO:20 form disulfide cross-links during expression, forming a multispecific multivalent biomolecule with one signaling-competent IL-2, one signaling-competent IL-4s and two signaling-deficient IL-2s. Other combinations of different single-chain polypeptides are fully embraced herein. These examples are merely illustrative of the variations in design and composition of multispecific multivalent biomolecule disclosed herein.

As will be described herein, the disclosure encompasses nucleic acids encoding the multispecific multivalent biomolecules disclosed herein, and components thereof, such that the multispecific multivalent biomolecules may be produced by expression by cells of the desired components (e.g., cross-linkable Fc region-containing single chain polypeptides that dimerize into multispecific multivalent biomolecules as disclosed herein, or other molecules disclosed herein). In other embodiments as described herein, production of the multispecific multivalent biomolecules may be achieved in vivo by administering to a patient or subject cells engineered to express the multispecific multivalent biomolecules or components thereof, or achieved in vivo by administering to the patient or subject a nucleic acid encoding multispecific multivalent biomolecules or components thereof, for example mRNA in a lipid nanoparticle, which on taking up by cells in the body, produce and export the multispecific multivalent biomolecules or components thereof, which in some embodiments said components may dimerize in vivo to form the desired multispecific multivalent biomolecules for treating a condition or disease such as described herein.

### Methods of Treatment

In one embodiment, a multispecific multivalent biomolecule disclosed herein produces an altered immunological response as compared to a biomolecule comprising the same γ-chain receptor cytokine in monomeric or multivalent form. In one embodiment, the altered immunological response results from altered signaling by the biomolecule. Examples of other altered immunological and other responses include, but are not limited to, altered pharmacokinetics, altered intracellular degradation, or altered in vivo half-life, or any combination thereof.

In one embodiment, the present disclosure provides a method for modulating the immune system of a subject, comprising administering to a subject in need thereof a multispecific multivalent biomolecule disclosed herein. In one embodiment, the multivalent biomolecule comprises a signaling-competent cytokine and an signaling-deficient cytokine, or variants thereof, from among the same or a combination of two or more common γ-chain receptor cytokine such as IL-2, IL-4, IL-7, IL-9, IL-15 or IL-21, and the method is used to modulate immune responses in the subject. In another embodiment, the multispecific multivalent biomolecule comprises a signaling-competent cytokine and an signaling-deficient cytokine, or variants thereof, from among the same or a combination of two or more common γ-chain receptor cytokine such as IL-2, IL-4, IL-7, IL-9, IL-15 or IL-21, and the method is used to activate immune responses in the subject. In another embodiment, the multispecific multivalent biomolecule comprises a signaling-competent cytokine and an signaling-deficient cytokine, or variants thereof, from among the same or a combination of two or more common γ-chain receptor cytokine such as IL-2, IL-4, IL-7, IL-9, IL-15 or IL-21, and the method is used to suppress immune responses in the subject. In one embodiment, the method can be used to treat cancer in the subject. In another embodiment, the method can be used to treat an autoimmune disease (e.g., systemic lupus erythematosus) or prevent transplant rejection in the subject. In some embodiments, the degree of mismatch between the donor and recipient of a transplant may provide the rationale for treatment with a multispecific multivalent biomolecule disclosed herein. Such uses are non-limiting examples of the therapeutic utilities of the multispecific multivalent biomolecules disclosed herein.

Examples of cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

Examples of autoimmune disease include, but are not limited to, achalasia, amyloidosis, ankylosing spondylitis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenia purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopeniarpura, type 1 diabetes, ulcerative colitis, uveitis, vasculitis, and vitiligo.

Organ transplant includes but is not limited to a solid organ transplant, a tissue transplant or a cellular transplant. Non-limiting examples include heart, lung, pancreas, intestine, nerve, tendon, skin, liver, kidney, bone, cornea, bone marrow and stem cells. In some embodiments, the method is used when rejection of the transplant is anticipated or detected. In some embodiments, the compatibility of the donor organ may be assessed and treatment with a multispecific multivalent biomolecule disclosed herein may be initiated.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treating”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition, or those in which the disease or condition is to be treated or prevented.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples can be assigned a relative activity value of 100%.

### Nucleic Acids

Thus, in some embodiments, a nucleic acid is provided encoding a single chain multispecific multivalent biomolecule disclosed herein. In some embodiments, a nucleic acid is provided encoding any one of SEQ ID NOs: 07, 08, 09, 11, 13, 15, 17, 19, 20, 22, 24, 26, 29, 31, 33, 35 or 37. In some embodiments, a vector is provided comprising a nucleic acid encoding a multispecific multivalent biomolecule disclosed herein. In some embodiments, a vector is provided comprising a nucleic acid encoding any one of SEQ ID NOs: SEQ ID NOs: 07, 08, 09, 11, 13, 15, 17, 19, 20, 22, 24, 26, 29, 31, 33, 35 or 37. Such nucleic acids and/or vectors are useful for preparing the multispecific multivalent biomolecules disclosed herein, such as the single-chain polypeptides comprising multiple cytokines sequences, or the Fc constructs comprising multiple cytokine sequences described herein, that may then be dimerized. It should be noted that in some embodiments, a nucleic acid described to be “encoding” a multispecific multivalent biomolecule (or syntactic variants thereof) is wherein the nucleic acid is encoding a component of the multispecific multivalent biomolecule, which upon expression, the components dimerize to form the multispecific multivalent biomolecule. It is understood within the disclosure that reference to a multispecific multivalent biomolecule encoded by a nucleic acid, or a nucleic acid encoding a multispecific multivalent biomolecule, includes wherein the nucleic acid encodes the components, which dimerize to form the multispecific multivalent biomolecule.

In other embodiments, such nucleic acids and vectors or plasmids comprising them, are useful for administration to a patient or subject such that, in one embodiments, such nucleic acids delivered to cells results in expression of the encoded multispecific multivalent biomolecule disclosed herein. In some embodiments, the nucleic acid (e.g., as a vector) is administered to the patient or subject (e.g., by parenteral administration). In some embodiments, cells are obtained from the patient, or from a donor or cell line, and in vitro or ex vivo exposed to a nucleic acid as disclosed herein, wherein such cells are subsequently administered to the subject or patient. In such embodiments, such cells produce at least one multispecific multivalent biomolecule disclosed herein or a component thereof. In some embodiments, the components of a multispecific multivalent biomolecule are produced by the cell, which dimerize (e.g., by dimerizable Fc regions) to produce an active (e.g., tetravalent) multispecific multivalent biomolecules. In some embodiments two or more nucleic acids encoding different multispecific multivalent cytokine components are administered to cells in order to produce tetravalent biomolecules comprising different components, such as SEQ ID NO: 7 and SEQ ID NO:20. In some embodiments, nucleic acids encoding different multispecific multivalent cytokine with dimerizing Fc introduced into a cells will produce a mixture of homodimeric and heterodimeric tetravalent biomolecules.

In some embodiments, such cells, whether exposed to a nucleic acid in vitro or ex vivo and administered, or exposed in vivo, provides for the production, in some embodiment, long term, production of the desired multispecific multivalent biomolecule for treatment of a condition or disease of the patient or subject.

Methods for administration of nucleic acids to cells or to patients or subjects are known in the art, and the present disclosure embraces any such method that achieves the desired purposes disclosed herein. In one embodiment, methods for delivering a mRNA to cells are described in de Picciotto et al., Selective activation and expansion of regulatory T cells using lipid encapsulated mRNA encoding a long-acting IL-2 mutein, Nature Communications 2022; 13:3866.

### Pharmaceutical Compositions

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease (e.g., cancer, auto-immune disease) or prolong the quality of life or survival of the subject being treated. Determination of a therapeutically effective amount is well within the capability of those skilled in the art. For example, for treatment of cancer to include effector T cells, a dosing regimen based on prior studies with IL-2 indicate a dose of 600,000 IU (0.037 mg) IL-2 per kg, administered IV three times a day for 14 doses, followed by a 9-day rest period and another 14 doses. The relative efficacy of the multivalent cytokines disclosed herein compared to, e.g., IL-2, will be factored into the dose and dosing regimen calculations for this or other indications. For treatment of autoimmune disease, a lower dose of IL-2 therapy is known in the art to be effective; the dose of a multispecific multivalent biomolecule disclosed here will be further adjusted based on the potency of the multispecific multivalent biomolecule compared to readily obtainable comparative data on monovalent cytokines. In one non-limiting example, a dose of multivalent IL-2 with potency equivalent to 1 million IU (0.062 mg) IL-2 given IV per day for 5 days, then once every 2 weeks for 6 months, is provided, the equivalent based on the efficacy of the multispecific multivalent biomolecules disclosed herein. As described herein, the multispecific multivalent biomolecules described herein will provide increased potency at equivalent or lower doses than monovalent cytokines currently on the market or in development.

Pharmaceutical compositions may comprise excipients, vehicles, diluents, carriers, and/or any other components to aid in the formulation, storage, aliquoting, vialing, sterilizing, packaging, distribution and/or administration of the multispecific multivalent biomolecule to a subject. Such pharmaceutical compositions may be administered by any route of administration appropriate for the intended use, typically but not necessarily intravenously or subcutaneously, or at a particular site in the body. Other routes include oral, intraarterial, intramuscular, parenteral, transmucosal, transdermal, or topical administration. The composition may be formulated as an immediate, controlled, extended or delayed release composition In some embodiments, a controlled release composition is injected or implanted subcutaneously or elsewhere in the body that slowly releases the biomolecule.

In some embodiments, the pharmaceutical composition is a controlled release delivery composition or device. In some embodiments the controlled release delivery composition or device provides for low levels of a multispecific multivalent cytokine to be delivered to sites in the subject or patient. In some embodiments, the controlled release delivery composition is biodegradable. Non-limiting examples of controlled release compositions include biodegradable scaffolds or hydrogels such as alginate, dextran, heparin, polyethylene glycol (PEG), poly(2-hydroxyethyl methacrylate), poly(vinyl alcohol), PEG-polyester copolymers, poly(N-isopropylacrylamide, and those described in Abune et al., Affinity Hydrogels for Protein Delivery, Trends Pharmacol Sci. 2021 April; 42(4): 300-312; Li et al., Designing hydrogels for controlled drug delivery, Nat Rev Mater. 2016 December; 1(12); Hennick et al., Controlled release of proteins from dextran hydrogels, Journal of Controlled Release, 1996; 39(1):47-55; Buwalda et al., Hydrogels for Therapeutic Delivery: Current Developments and Future Directions, Biomacromolecules 2017; 18:316-330; and Nagy et al., Weekly injection of IL-2 using an injectable hydrogel reduces autoimmune diabetes incidence in NOD mice, Diabetologia 2021 January; 64 (1): 152-158. The foregoing references are incorporated herein in their entireties.

As noted herein, another controlled release delivery method is to deliver a nucleic acid encoding a multispecific multivalent cytokine to cells within the subject or patient, wherein such cells express and release the multispecific multivalent cytokine at desirable levels for effective treatment. Such levels may be guided by the teaching herein and that for cytokines therapies known in the art.

In one embodiment, for any preparation used in the methods disclosed herein, the therapeutically effective amount or dose can be estimated initially from in vitro assays. For example, a dose can be formulated in animal models and such information can be used to determine useful doses more accurately in humans. In another embodiment, toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1]. In one embodiment, the useful dose (or need) for mitigating or treating transplant rejection may be evaluated in vitro using a mixed lymphocyte reaction assay.

The dose and dosing regimen are selected to provide an efficacious treatment for the subject or patient in need, and is tailored to the particular disease and/or other conditions of the subject. The dose level, dosing frequency (e.g., once, twice or three times a day, or less frequently such as twice a week, once a week, every 2, 3 or 4 weeks, for example) will be determined by the pharmacokinetics, severity of disease, potential side effects, tolerability, and resolution of the disease and/or symptoms of the subject. The duration of dosing, possible dosing holidays, and other aspects of the dosing regimen will be determined by the healthcare professional based on the foregoing and other relevant medical information.

In some embodiments, a nucleic acid such as a DNA or mRNA or a vector or plasmid encoding a multispecific multivalent cytokine or component thereof is administered to a subject or patient, or to cells from the subject or patient or from a donor or cell line. In some embodiments, the nucleic acid is administered in a nanoparticle. In some embodiments, the nucleic is administered in a lipid nanoparticle. Pharmaceutical compositions for delivering nucleic acids to cells or to a patient or subject are known in the art; non-limiting examples include that described by Sabnis, S. et al., A novel amino lipid series for mRNA delivery: Improved endosomal escape and sustained pharmacology and safety in non-human primates. Mol. Ther. 2018; 26(6):1509-1519; Kim et al., 2019, Adv Mater (49)e1903637; Ickenstein et al., 2019, Expert Opin Drug Deliv 16(11): 1205-1226); Zhao et al., Lipid Nanoparticles for Gene Delivery, Adv Genet 2014; 88:13-36; Kulkarni et al., Design of lipid nanoparticles for in vitro and in vivo delivery of plasmid DNA, Nanomedicine: Nanotechnology, Biology, and Medicine 2017; 13:1377-1387; Eygeris et al., Chemistry of Lipid Nanoparticles for RNA Delivery, Acc Chem Res 2022; 55(1):2-12; Hou et al., Lipid Nanoparticles for mRNA Delivery, Nature Review Materials 2021; 6:1078-1094; and Algarni et al., In vivo delivery of plasmid DNA by lipid nanoparticles: the influence of ionizable cationic lipids on organ-selective gene expression, Biomaterials Science 2022; 10:2940-2952, all of which are incorporated herein by reference. As noted herein, dimerizable components of a multispecific multivalent cytokine may be produced by a cell and the components dimerize (e.g., by dimerizable Fc regions) to form a tetravalent multispecific multivalent cytokine.

In another one embodiment, the activity of a multispecific multivalent biomolecule disclosed herein on elevating T_{reg}s in a patient undergoing treatment may be assessed by determining the T_{reg} abundance in a blood sample from the patient, which may be determined over time, e.g., during and after the treatment period. In some embodiments, titration of the dose level in a patient is carried out by measuring T_{reg} levels periodically and adjusting the dose or dose regimen. In some embodiments, determining the optimal effective dose or dose regimen of a multispecific multivalent biomolecule in a clinical study, may be carried out by conducting a dose response study to identify the highest dose of multispecific multivalent biomolecule that expands the T_{reg} population without expanding other T cell populations such as helper T cells and/or NK cells. Such monitoring of activity may be provided during clinical development of a multispecific multivalent biomolecule, or recommended monitoring for patients receiving treatment.

As used herein, the terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Throughout this application, various embodiments of the present disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the disclosure and variations thereof are described. This description is not intended to be limiting and changes in the components, sequence of steps, and other variations would be understood to be within the scope of the present disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

### EXAMPLES

### Materials and Methods

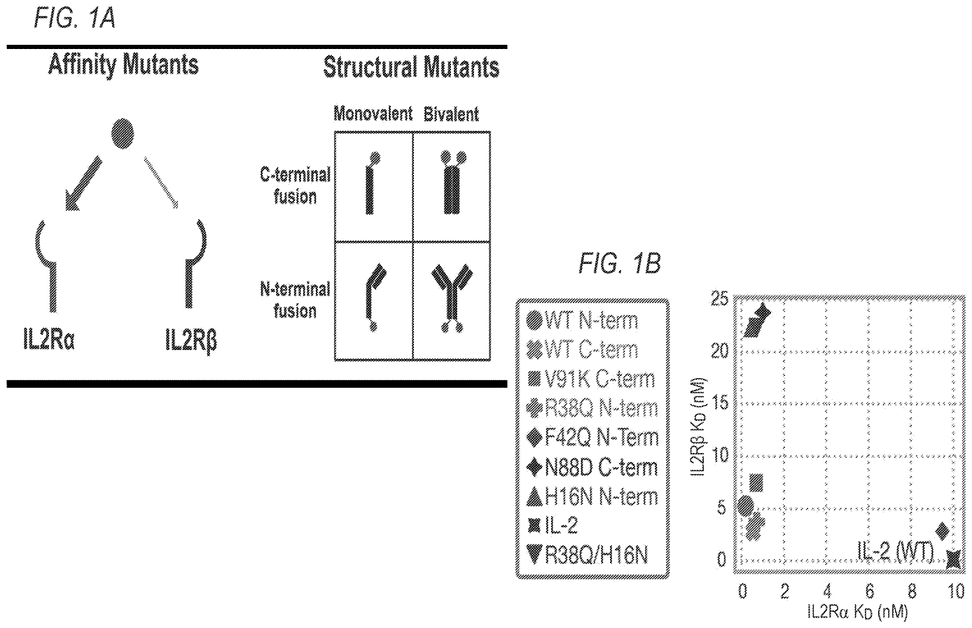

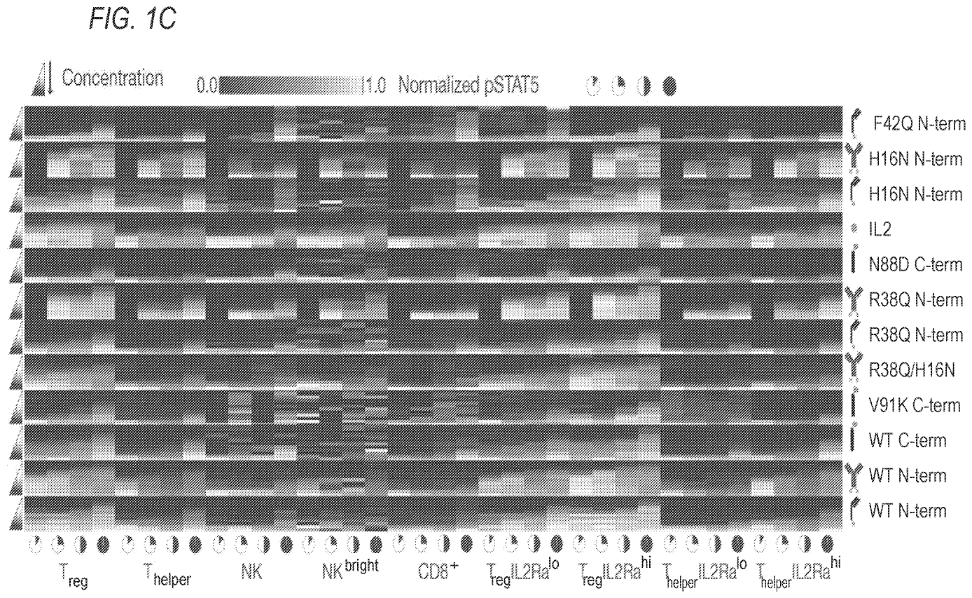

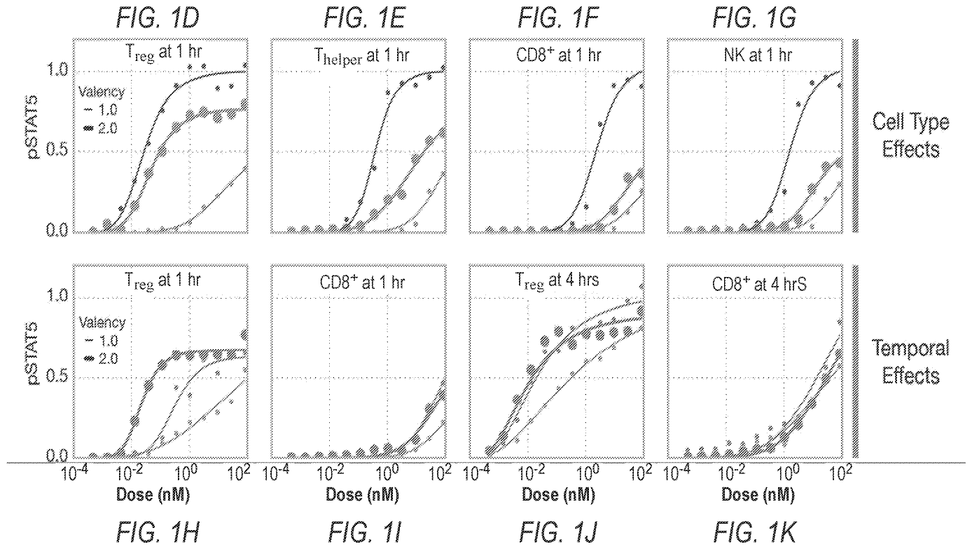

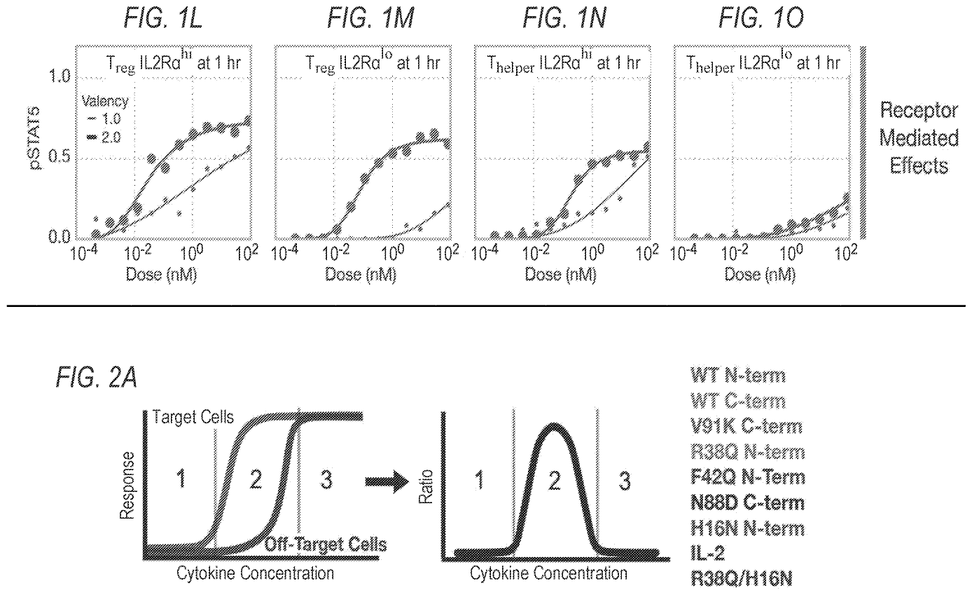

Receptor abundance quantitation, octet binding assays, expression of recombinant bivalent and monovalent IL-2 muteins (FIGS. 1-4), and measurement of those muteins' signaling in PBMCs were performed as described in Farhat, A. M. et al. Modeling cell-specific dynamics and regulation of the common gamma chain cytokines. Cell Rep 35, 109044 (2021).

### Receptor Abundance Quantitation

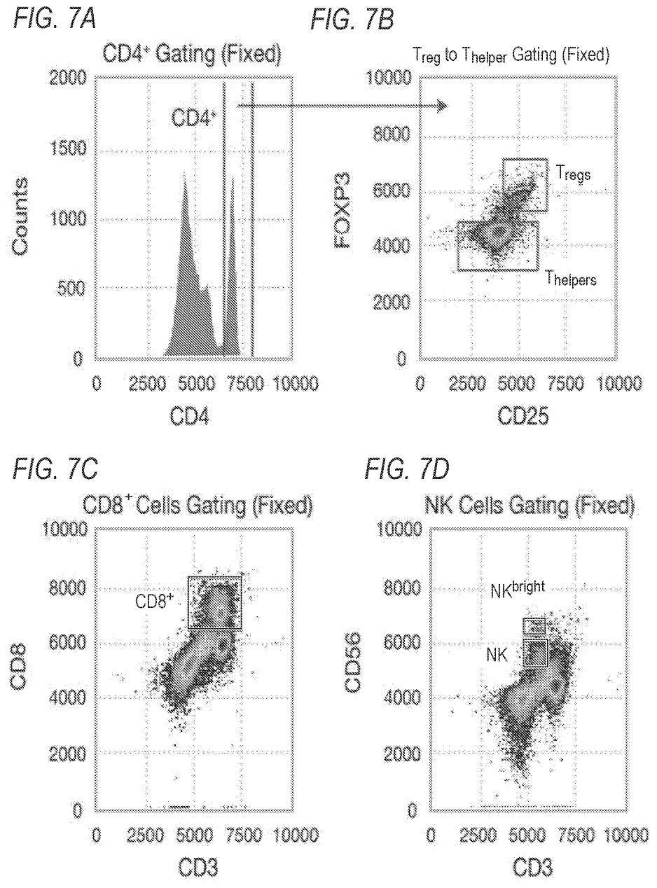

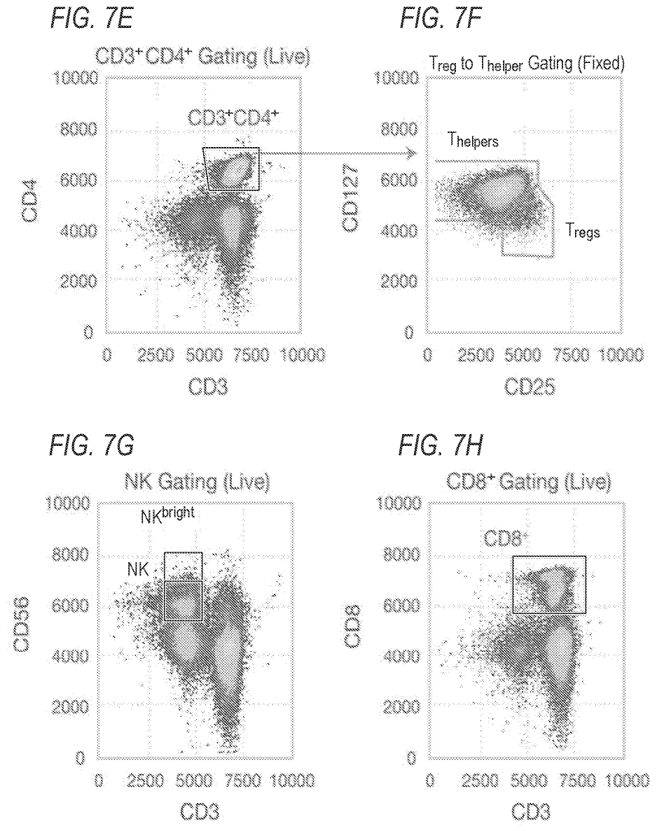

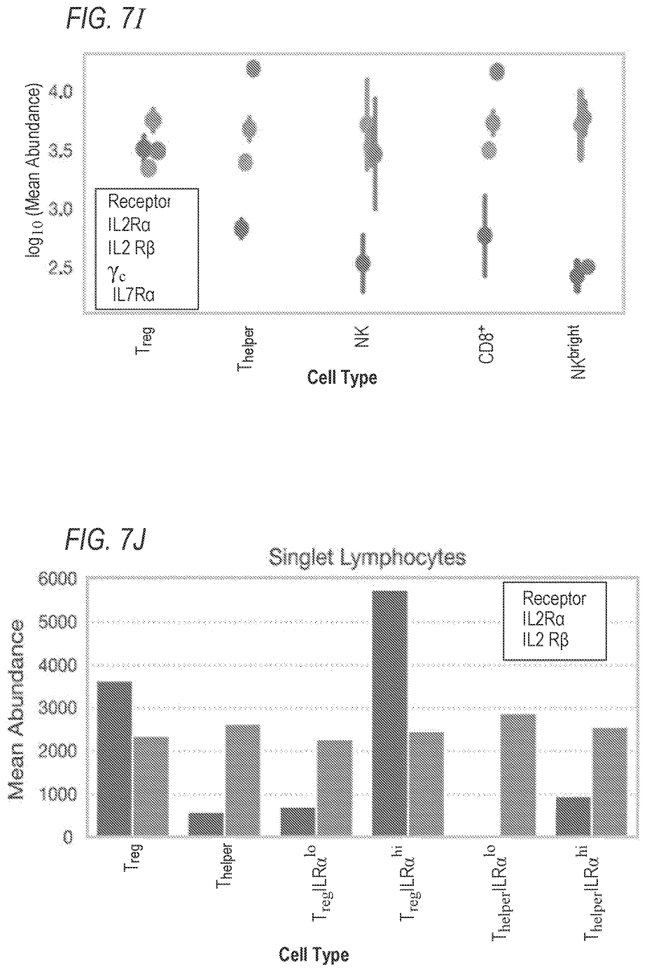

Receptor quantitation data was gathered as described previously in Farhat et al. (op. cit); the preprocessing of fluorescence measurements, population gating, and receptor abundance calculations were performed using these data. To quantify the number of antibodies bound to cells and to standard beads, the fluorescence intensity of isotype controls was subtracted from the signal from matched receptor stains and then calibrated using the two lowest quantitation standards. Cell gating was conducted as shown in FIG. 7I, A to H. The geometric means of replicates were calculated to summarize the results.

pSTAT5-Based Measurement of IL-2 and IL-15 Signaling in PBMCs

Cryopreserved PBMCs (ATCC, PCS-800-011, Lot #81115172) were thawed to room temperature and slowly diluted with 9 mL pre-warmed RPMI-1640 (Corning, 10040 CV) supplemented with 10% FBS (VWR, 97068-091, lot #029K20) and Penicillin/Streptomycin (Gibco, 15140122). Media was removed, and cells were brought to 3×10⁶ cells/mL, distributed at 300,000 cells per well in a 96-well V-bottom plate, and allowed to recover 2 hrs at 37° C. in an incubator at 5% CO₂. IL-2 (R&D Systems, 202-IL-010) or IL-15 (R&D Systems, 247-ILB-025) were diluted in RPMI-1640 in the absence of FBS. These dilutions were then added to the concentrations indicated. To quantify STAT5 phosphorylation, the media was taken away, and cells were fixed using 100 μL of 10% formalin (Fisher Scientific, SF100-4) for 15 mins at room temperature. Formalin was removed from the cells, and the PBMCs were placed on ice. They were then suspended in 50 μL of cold methanol (−30° C.). PBMCs were then kept at −30° C. overnight. PBSA was used to wash the cells twice. The cells were then split into two identical plates and stained with fluorescent antibodies for 1 hr at room temperature in darkness using 50 μL of antibody panels 4 and 5 per well. Cells were suspended in 100 μL PBSA per well, and beads to 50 μL, and analyzed on an IntelliCyt iQue Screener PLUS with VBR configuration (Sartorius) using a sip time of 35 secs and beads 30 secs. Compensation of measured fluorescent values was calculated as detailed above. Gating of cell populations was performed as shown in FIG. 7, and the median pSTAT5 level was calculated for each population in each well.

### Recombinant Proteins

The Expi293 expression system was used to express IL-2/Fc fusion proteins. Expression was conducted as prescribed by the manufacturer instructions (Thermo Scientific). Proteins were formulated as the Fc of human IgG1 fused at its N- or C-terminus to human IL-2 using a (G₄S)₄ linker (SEQ ID NO:06). C-terminal lysine residues of human IgG1 were not included in C-terminal fusions. The AviTag sequence GLNDIFEAQKIEWHE was added to the Fc terminus which did not contain IL-2. Fc mutations which prevented dimerization were introduced into the Fc sequence for monovalent muteins (Ishino, T. et al. Engineering a monomeric Fc domain modality by N-glycosylation for the half-life extension of biotherapeutics. J Biol Chem 288, 16529-16537 (2013)). MabSelect resin (GE Healthcare) was used to purify protein. Biotinylation of proteins was conducted using BirA enzyme (BPS Biosciences) according to manufacturer instructions. Extensive buffer-exchanging into phosphate buffered saline (PBS) was conducted using Amicon 10 kDa spin concentrators (EMD Millipore). The sequence which was used to express the IL2Rβ/γ Fc heterodimer was the same as that of a reported, active heterodimeric molecule (patent application US20150218260A1); a (G₄S)₂ linker was added between the Fc portion and each receptor ectodomain. The Expi293 system was used to express the protein, which was subsequently purified on MabSelect resin as above. The IL2Rα ectodomain was generated to include a C-terminal 6×His tag and then purified on Nickel-NTA spin columns (Qiagen) according to manufacturer instructions.

pSTAT5-Based Measurement of Tetravalent IL-2 Signaling in PBMCs

Cryopreserved PBMCs (UCLA Virology Core, sex of donors unknown) were thawed to room temperature and slowly diluted with 9 mL pre-warmed RPMI-1640 (Corning, 10040 CV) supplemented with 10% FBS (VWR, 97068-091, lot #029K20) and Penicillin/Streptomycin (Gibco, 15140122). Media was removed, and cells were brought to 3×10⁶ cells/mL, distributed at 300,000 cells per well in a 96-well V-bottom plate, and allowed to recover 2 hrs at 37° C. in an incubator at 5% CO₂. IL-2 (Peprotech, 200-02-50 mg) and tetravalent IL-2 (expressed and purified as described below) were diluted in RPMI-1640 without FBS and added to the indicated concentrations. Cells were stained with antibodies from panel 1 described below. To measure pSTAT5, media was removed, and cells fixed in 100 μL of 4% paraformaldehyde (PFA, Election Microscopy Sciences, 15714) diluted in PBS for 15 mins at room temperature.

PFA was removed, cells were gently suspended in 100 μL of cold methanol (−30° C.). Cells were stored overnight at −30° C., and then washed twice with 0.1% bovine serum albumin (BSA, Sigma-Aldrich, B4287-25G) in PBS (PBSA), and stained 1 hr at room temperature in darkness using antibody panel X with 40 μL per well. Cells were then washed twice with 0.1% PBSA and resuspended in 150 μL PBSA per well. Cells were analyzed on a BD FACSCelesta flow cytometer. Populations were gated (as shown in FIG. 7, A to H), and the median pSTAT5 level was extracted for each population in each well. Wells with fewer than 1000 cells were excluded from analysis (resulted in removal of 6 wells over 5 experimental replicates.

### Tetravalent IL-2 Expression

Proteins were expressed as human IgG1 Fc-fused at the N- or C-terminus to mutant human IL-2 through a flexible (G₄S)₄ linker. C-terminal fusions omitted the C-terminal lysine residue of human IgG1. In monovalent R38Q/H16N variants, Fc mutations to prevent dimerization were introduced into the Fc sequence. In R38Q/H16N variants, each IL-2 fused via the 20 amino acid long linker to the Fc domain contained R38Q and H16N mutations to reduce the IL-2's affinity with which it binds IL2Rβ. In bitargeted variants, one IL-2 included R38Q/H16N mutations, and the other IL-2 fused to the Fc domain included V91K/D20A/M104V mutations to ablate binding to IL2Rβ. In bivalent bitargeted IL-2, Fc mutations were included to prevent Fc dimerization. Plasmid DNA prepared by maxi-prep (Qiagen, 12162) were transfected into adherent HEK293T cells using Lipofectamine 3000 (Thermo-Fisher, L3000008) in 15 cm dishes in DMEM (Corning, 15017 CV) supplemented with GlutaMax (Gibco, 35050061) and 10% FBS. Media was exchanged after 24 hrs with fresh DMEM supplemented with GlutaMax and 5% ultra-low IgG FBS (Thermo-Fisher, A3381901). Media was harvested after an additional 72 hrs. Media was incubated in the presence of Protein A/G Plus Agarose resin (Santa Cruz Biotechnology, sc-2003) overnight. The following day, the media-resin mixture was centrifuged, and the supernatant discarded. Resin was washed with PBS five times or until protein was no longer detected in supernatant by UV-Vis using a NanoDrop One Spectrophotometer (Thermo-Fisher, ND-ONE-W). IL-2 was eluted from resin using 0.1M glycine, pH 2.3, into 2M Tris-HCl, pH 8. IL-2 was then buffer exchanged into PBS for storage at −80° C. Concentration was determined by BCA assay and confirmed using an IgG1 ELISA.

### Octet Binding Assays

An Octet RED384 (ForteBio) was used to measure the binding affinity of each IL-2 mutein. Monomeric, biotinylated IL-2/Fc fusion proteins were loaded to Streptavidin biosensors (ForteBio) at roughly 10% of saturation point and allowed to equilibrate for 10 min in PBS+0.1% bovine serum albumin (BSA). Up to 40 min of association time in IL2Rβ/γ titrated in 2× steps from 400 nM to 6.25 nM, or IL2Rα from 25 nM to 20 μM, which was followed by dissociation in PBS+0.1% BSA. A zero-concentration sample was included in each measurement and served as a negative control/reference signal. The affinity quantification experiments were performed in quadruplicate across two days. Binding of IL-2 to IL2Rα on its own did not fit to a simple binding model; K_{D} was calculated using equilibrium binding within each assay for this case. IL2Rβ/γ binding data fit a 1:1 binding model; thus, in these cases on-rate (kₒₙ), off-rate (k_{off}) and K_{D} were determined by fitting to the entire binding curve. The average of each kinetic parameter across all concentrations with detectable binding (typically 12.5 nM and above) was used to calculate K_{D}.

### CD25 Measurement in T_{reg}s and ILC2s

Cryopreserved PBMCs (UCLA Virology Core, sex of donors unknown) from each donor were thawed to room temperature and slowly diluted with 9 mL pre-warmed RPMI-1640 (Corning, 10040 CV) supplemented with 10% FBS (VWR, 97068-091, lot #029K20) and Penicillin/Streptomycin (Gibco, 15140122). Media was then removed and PBMCs were washed with ice cold 1% bovine serum albumin (BSA, Sigma-Aldrich, B4287-25G) in PBS (PBSA). PBMCs were then stained for one hour at 4° C. in a cocktail of anti-lineage FITC (Invitrogen, 22-7778-72), anti-FcεR1 FITC (Biolegend 334608), anti-CD25 APC/Fire 810 (Biolegend 356150), anti-CD127 Brilliant Violet 421 (Biolegend 351310), and anti-CRTH2 Brilliant Violet 605 (Biolegend 350122), (panel 2 below) all at a dilution of 1:20 in PBSA except for the anti-lineage antibody, which was diluted 1:10. Cells were then washed once with cold PBSA and once with cold PBS, then fixed in 2% paraformaldehyde (PFA) at room temperature for 15 minutes. Paraformaldehyde (PFA, Election Microscopy Sciences, 15714) diluted in PBS for 15 mins at room temperature. PFA was then removed, and cells were washed once with PBS. Cells were then resuspended in ice cold methanol and incubated on ice for 30 minutes. Cells were then washed with PBS and resuspended in anti-Foxp3 Alexa Fluor 647 (Biolegend 320114) diluted 1:20 in PBSA for 1 hour. Cells were then washed twice with PBSA before being resuspended in PBSA for analysis on a BD FACSCelesta flow cytometer. Populations were gated as shown in supplementary figures, and the median IL2Rα abundance was extracted for each population.

### Statistical Analysis

The number of replicates performed for each experimental measurement, and the values of confidence intervals are described in corresponding figure captions. N is used to describe the number of times a particular experiment was performed. Flow cytometry experiments performed using initial panel of monovalent and bivalent cytokines (FIGS. 1-4) were performed on hPBMCs were conducted using separate experimental replicates on cells gathered from a single donor. Each replicate of the flow cytometry signaling experiments in FIGS. 5 and 6 were conducted using hPBMCs from different donors. To quantify population-level flow cytometry measurements for both signaling and receptor quantitation experiments, the mean fluorescent intensity (MFI) of a gated population was measured. Compensation to remove fluorescent spectral overlap was performed for each experimental measurement. Subtraction of either negative controls or cells treated with isotype antibodies was performed on signaling and receptor quantitation data respectively to remove background signal. Cells which were measured to display fluorescent intensities above 1,000,000 were excluded from analysis during signaling experiments. Pearson correlation coefficients (R2) values were used to describe model accuracy when predicting signaling response to IL-2 and IL-2 muteins. The Kₓ*parameter was fit with least-squares fitting using the Broyden-Fletcher-Goldfarb-Shanno minimization algorithm as implemented in SciPy.

### Antibodies

The following antibodies were used to quantify receptor abundances, as well as to perform initial pSTAT5 response quantification in PBMCs can be found in Farhat et al. (op. cit).

| Antibody | Source | Identifiers | Panel |
|---|---|---|---|
| Anti-FoxP3, | BioLegend | Cat #: 320216; Clone: 206D; RRID: AB_439801 | 1 |
| Pacific Blue | | | |
| Anti-CD3, | BioLegend | Cat #: 300328; Clone: HIT3a; RRID: AB_1575008 | 1 |
| PerCP-Cy5 | | | |
| Anti-CD4, PE | BioLegend | Cat #: 300508; Clone: RPA-T4; RRID: AB_314076 | 1 |
| Anti-CD8a, | BioLegend | Cat #: 344716; Clone: SK1; RRID: AB_10549301 | 1 |
| Alexa Fluor 488 | | | |
| Anti-CD56, | BioLegend | Cat #: 318334; Clone: GCD56; RRID: AB_2561912 | 1 |
| BV605 | | | |
| Anti-pSTAT5, | BD Biosciences | Cat #: BDB566978; Clone: 47; RRID: | 1 |
| R718 | | AB_2869984 | |
| Anti-CD25, | BD Biosciences | Cat #: 563701; Clone: M-A251; RRID: | 1 |
| BV786 | | AB_2744338 | |
| Anti-CD25, | BioLegend | Cat #: 356150; Clone: M-A251; RRID: | 2 |
| APC-Fire 810 | | AB_2876679 | |
| Anti-CD127, | BioLegend | Cat # 351310 Clone: A019D5; RRID: | 2 |
| BV421 | | AB_10960140 | |
| Lineage Cocktail, | Invitrogen | Cat # 22-7778-72; Clones: | 2 |
| FITC | | UCHT1; HCD14; 3G8; HIB19; 2H7; HCD56; | |
| | | RRID: AB_1311229 | |
| Anti-CRTH2, | BioLegend | Cat #: 350122; Clone: BM16; RRID: AB_2566760 | 2 |
| BV605 | | | |
| Anti-FoxP3, | BioLegend | Cat # 320114; Clone: 206D; RRID: AB_439754 | 2 |
| Alexa 647 | | | |
| Anti-FcεR1, | Biolegend | Cat # 334608; Clone: AER-37; RRID: AB_1227653 | 2 |
| FITC | | | |
| Anti-IL-2 | R&D Systems | Cat # MAB202-SP; Clone 5334; RRID: | N/A |
| | | AB_2264789 | |

### Newly Created Materials

All novel IL-2 muteins were synthesized as described above, and contained the mutations as described above. No restrictions on access to these materials are noted.

### Binding Model

The model was formulated as described in Tan et al. (Tan, Z. C. & Meyer, A. S. A general model of multivalent binding with ligands of heterotypic subunits and multiple surface receptors. Math Biosci 108714 (2021)). The monomer composition of a ligand complex was represented by a vector θ=(θ₁,θ₂, . . . , θ_{N}_{_{L}}), where each θᵢ was the fraction of monomer ligand type i out of all monomers on that complex. Let Ce be the proportion of the 0 complexes in all ligand complexes, and Θ be the set of all possible θ's. Σ_{θ∈Θ}C_{θ}=1.

The binding between a ligand complex and a cell expressing several types of receptors can be represented by a series of qᵢⱼ. The relationship between qᵢⱼ's and θᵢ is given by θᵢ=qᵢ₀+qᵢ₁+ . . . +q_{iN}_{_{R}}. Let the vector qᵢ=(gᵢ₀, qᵢ₁, . . . , q_{iN}_{_{R}}), and the corresponding 0 of a binding configuration q be θ(q). For all i in {1, 2, . . . , N_{L}}, we define ψᵢⱼ=R_{eq,j}K_{a,ij}Kₓ* where j={1, 2, . . . , N_{R}} and ψᵢ₀=1. The relative number of complexes bound to a cell with configuration q at equilibrium is:

$${v}_{q , eq} = \frac{{L}_{0} {C}_{θ ( q )}}{{K}_{x}^{*}} {\prod }_{{i = 1}_{j = 0}}^{{j = {N}_{R}}^{i = {N}_{L}}} {ψ}_{ij}^{{q}_{ij}} {\prod }_{i = 1}^{{N}_{L}} ( \begin{matrix}{θ}_{i} \\ {q}_{i}\end{matrix} ) .$$

### Then we can Calculate the Relative Amount of Bound Receptor n as

$${R}_{bound , n} = \frac{{L}_{0}}{{K}_{x}^{*}} {\sum }_{θ ∈ Θ} {C}_{θ} [ {\sum }_{i = 1}^{{N}_{L}} \frac{{ψ}_{in} {θ}_{i}}{{\sum }_{j = 0}^{{N}_{R}} {ψ}_{ij}} ] {\prod }_{i = 1}^{{N}_{L}} {( {\sum }_{j = 0}^{{N}_{R}} {ψ}_{ij} )}^{{θ}_{i}} .$$

By mass conservation, (R_{tot,n}=R_{eq,n}+R_{bound,n}), we can solve R_{eq,n} numerically for each type of receptor.

### Application of Multivalent Binding Model to IL-2 Signaling Pathway

Each IL-2 molecule was allowed to bind to one free IL2Rα and one IL2Rβ/γ_{c} receptor. Initial IL-2-receptor association proceeded with the known kinetics of monomeric ligand-receptor interaction (table S1). Subsequent ligand-receptor binding interactions then proceeded with an association constant proportional to available receptor abundance and affinity multiplied by the scaling constant, Kₓ*, as described above. To predict the pSTAT5 response to IL-2 stimulation, we assumed that pSTAT5 is proportional to the amount of IL-2-bound IL2Rβ/γ_{c}, because complexes which contain these species actively signal through the JAK/STAT pathway. Scaling factors converting from predicted active signaling species to pSTAT5 abundance were fit to experimental data on a per-experiment and cell type basis. A single Kₓ* value was fit for all experiments and cell types.

### CITE-Seq Marker Selectivity Analysis

To assist in identifying possible markers to increase IL-2 selectivity towards T_{reg}s, a publicly available Cellular Indexing of Transcriptomes and Epitopes by sequencing (CITE-seq) dataset containing data gathered from human PBMCs was analyzed. Only RNA transcripts encoding cell membrane extracellular-facing proteins were included. We first analyzed the data by determining the Wasserstein distance and Kullback-Leibler divergence of markers and RNA measured in T_{reg}s against the distribution of these markers displayed by all other cells. We also analyzed the data using a ridge classification model, where all markers and RNA sequences were used by the model to distinguish between T_{reg}s and all other cell types.

Markers of interest were then used in conjunction with the binding model to determine whether they could confer selectivity, using the CITE-seq data to inform the number of markers per cell. Conversion factors for calculating marker abundance from CITE-seq marker and mRNA reads were estimated using proportional conversions from the data to previously experimentally determined marker abundances. Single cell marker abundances were calculated for 1000 cells at a time, and the ratio of T_{reg} signaling to off-target signaling was calculated. To simulate bispecific binding, two distinct binding domains for each ligand were modeled, one for IL-2, with affinity for IL2Rα and IL2Rβ/γ_{c}, and the other for the marker of interest. The ligand affinities were varied while defining selectivity as the summed T_{reg} signaling divided by the signaling across all off-target cell populations. After finding IL2Rα to be the optimal epitope for increasing selectivity, we sought to explore the effects of increasing valency by doubling the number of binding domains per ligand.

### Tensor Factorization

Before decomposition, the signaling response data was background subtracted and variance scaled across each cell population. Non-negative canonical polyadic decomposition was performed using the Python package TensorLy, using the HALS algorithm with non-negative SVD initialization.

### Example 1. Systematic IL-2 Variant Profiling Reveals Multiple Determinants of Response

To explore how IL-2 mutations affect signaling across immune populations, we stimulated peripheral blood mononuclear cells (PBMCs), collected from a single donor, with 13 IL-2 muteins (FIGS. 1, A and B, and table S1). Our panel included several IL-2 muteins previously developed to confer enhanced T_{reg}-selective signaling. In addition to changes in receptor affinities, the muteins included some variation in structural features: Fc fusion at either the C- or N-terminus, which has been shown to alter receptor-interaction kinetics, and fusion to Fc in both monomeric or dimeric formats. We previously profiled six of the eleven IL-2 muteins; however, we both expanded this previous panel while also adding dimeric Fc fusions, thus greatly expanding the scope of IL-2 engineering approaches surveyed. Our panel includes several muteins previously published or clinically developed for their T_{reg} selectivity-monomeric N88D and dimeric R38Q/H16N were previously developed by Amgen and Otsuka Pharmaceuticals, respectively. Our panel was also designed to feature IL-2 variants whose affinities for IL2Rα and IL2Rβ span the range of currently available T_{reg} selective affinity mutants (FIG. 1B).

TABLE 1
IL-2 Variant Affinities for IL-2R Subunits

| Ligand | IL2Rα K_{D} (nM) | IL2Rβ/γ_{c} K_{D} (nM) |
|---|---|---|
| WT IL-2 | 10.0 | 0.133 |
| WT N-term | 0.19 | 5.30 |
| WT C-term | 0.54 | 3.04 |
| V91K C-term | 0.69 | 7.56 |
| R38Q N-term | 0.71 | 4.00 |
| F42Q N-Term | 9.48 | 2.81 |
| N88D C-term | 1.01 | 24.0 |
| H16N N-term | 0.43 | 22.4 |
| R38Q/H16N | 0.71 | 22.4 |

The panel of IL-2 variants was used to stimulate cells from a single donor at four time points using 12 treatment concentrations. The PBMCs were then stained for canonical cell type markers and phosphorylated STAT5, a commonly used read-out of IL-2 signaling response, allowing us to separate signaling response by cell type. Five different cell types—T_{reg}, helper T (Tₕₑₗₚₑᵣ), CD8⁺, NK, and NK CD56^{bright} (NK^{bright}) cells—were gated and quantified (FIG. 7, A to D). T_{reg} and Tₕₑₗₚₑᵣ cells were further dissected into low, average, and high IL2Rα abundance by isolating subpopulations using three logarithmically spaced bins (FIG. 7J). For a surface-level visualization of the effects of time, cell type, receptor abundance, ligand format, ligand affinity, and concentration, we organized our signaling data into a heatmap (FIG. 1C). The complexity of the data demanded closer examination.

We selectively highlighted several dose-response curves to demonstrate the importance of our comprehensive characterization (FIG. 1, D to O). First, as expected, we found that the affinity with which each IL-2 interacted with receptors divided responses (FIG. 1, D to G); for example, wild-type (WT) IL-2 most potently activated all cell types, as expected given it bound to IL2Rβ with the greatest affinity (FIGS. 1, B and D to G). Valency also had a prominent effect on signaling response; the bivalent Fc fusion form increased sensitivity and potency of response across all cell types.

Temporal dynamics also affected response characteristics (FIG. 1, H to K). For example, we found that C- or N-terminus Fc-fused IL-2 demonstrated distinct responses in T_{reg}s at 1 hour of treatment but shared responses after 4 hours of treatment (FIG. 1, H to K). Temporal effects are likely influenced by receptor-mediated endocytosis of IL-2 receptor subunits and transcriptional changes arising from IL-2 signaling.

Finally, we found that receptor abundance interacted with cell identity to alter response (FIG. 1, L to O). T_{reg} populations with high amounts of IL2Rα strongly responded to monovalent H16N N-term, and the bivalent form moderately enhanced this response. However, in the IL2Rα^{lo} T_{reg} cells, the effect of bivalency was even greater; only bivalent H16N induced a significant response. IL2Rα^{high} Tₕₑₗₚₑᵣ cells also showed a moderate increase in potency with bivalency, like the IL2Rα^{high} T_{reg} cells, but the IL2Rα^{lo} population showed no distinction between the monovalent and bivalent fusions. Thus, immune populations are further subdivided by receptor abundance into subpopulations with distinct cellular responses.

In total, the dynamics of response, cell type, concentration, ligand affinity, Fc fusion valency, and Fc fusion orientation all play roles in determining cellular response. These determinants interact in unique and often counter to intuition, requiring a more comprehensive accounting of their effects.

### Example 2. Ligand Valency and Affinity Interact to Form Unique Cell Type Selectivity Profiles

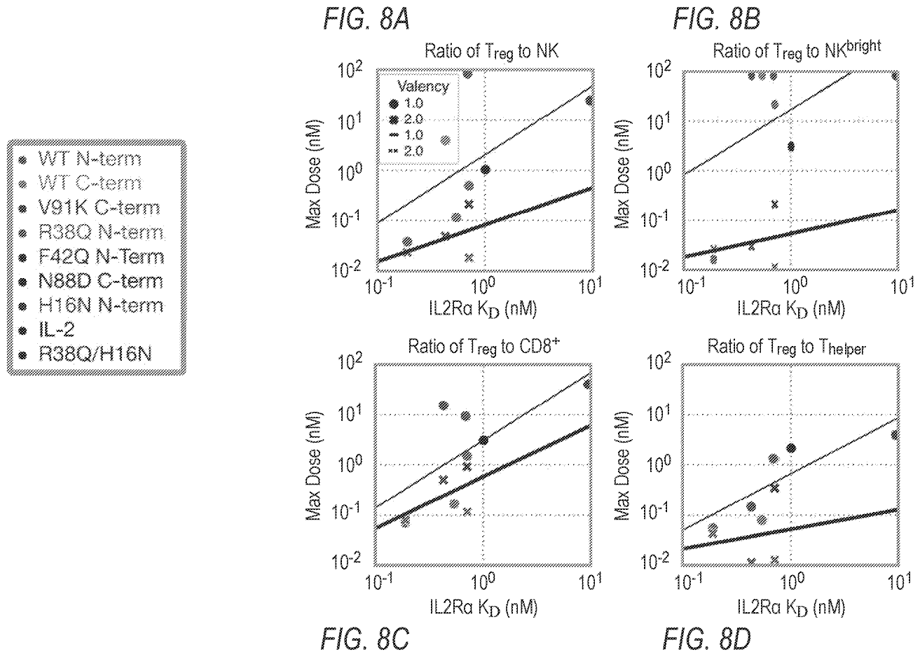

Given the coordinated importance of time, ligand valency, ligand affinity, cell type, and receptor expression, we next sought to focus on how ligand format affected T_{reg} selectivity. The selectivity of IL-2 for specific cell types corresponds closely to its therapeutic potency and potential toxicities. Therefore, we sought to better understand the relationship between T_{reg} selectivity and ligand properties (FIG. 2, A to I). First, we plotted the ratio of STAT5 phosphorylation (fit by a Hill curve) in T_{reg}s to that of off-target cells for each ligand across our concentration range and saw that the shape of each selectivity curve varied substantially for each ligand and off-target cell type that was considered (FIGS. 2, A, B, D, F, and H). T_{reg} selectivity quantified against CD8⁺, NK, or NK^{bright} cells most prominently separated bivalent from monovalent ligands, with bivalent muteins being most selective for T_{reg}s at lower concentrations. The selectivity demonstrated by bivalent muteins at lower doses can also be quantified by observing their lower T_{reg} activation EC₅₀ values and relatively unchanged off-target EC₅₀ values (Table 2). Because IL2Rα affinity varied widely between our IL-2 mutants and is a known T_{reg} selectivity regulator, we sought to understand how affinity differences contribute to T_{reg} selectivity. We plotted IL2Rα affinity against the peak T_{reg} selectivity observed across concentrations (FIGS. 2, C, E, G, and I). Due to the high abundance of IL2Rα displayed by T_{reg}s (FIG. 7I), we expected to see a positive correlation between IL2Rα affinity and peak T_{reg} selectivity. However, we saw that this relationship varied in a cell type- and valency-dependent manner. When considering either CD8⁺ or NK cells, decreasing IL2Rα affinity led to decreases in the peak T_{reg} selectivity of monovalent muteins but also, somewhat unexpectedly, little relationship with the bivalent selectivity peaks (FIGS. 2, C, E, and F). When considering Tₕₑₗₚₑᵣ populations, which have greater amounts of IL2Rα than CD8⁺ and NK cells, we observed that decreases in IL2Rα affinity led to increases in maximum selectivity for both monovalent and bivalent muteins (FIG. 2I). Expectedly, the mutein concentration at which the maximum T_{reg} selectivity occurred was higher for ligands with weaker IL2Rα affinity across all cell types (FIG. 8, A to D). In total, affinity and valency affected the selectivity profiles across ligand doses in distinct yet intertwined manners. To understand these relationships, a method mapping each of these factors simultaneously is needed.

TABLE 2
IL-2 variants' EC50 values for each immune cell subtype. The EC50
of each IL-2 mutein for immune cell subtype as determined using STAT5
phosphorylation. All EC50 values are reported in nM units and were
determined by fitting a Hill function to an experimental dose-response
curve. EC50 values greater than the dose range tested are reported
here as “N/A”. The valency of each ligand is reported next
to the ligand's name (Mono, monovalent; Biv, bivalent). Signaling
data was gathered using PBMCs harvested from a single donor, and
three technical replicates were performed. Hill curves were fit to
all experimental replicates simultaneously for each ligand and cell
population. Each experiment was repeated sing one donor, and four
technical replicates were performed, where cells from a single blood
draw were split, thawed, and tested on different days. (N = 4).

| Ligand | CD8 | NK | NK^{bright} | Tₕₑₗₚₑᵣ | T_{reg} |
|---|---|---|---|---|---|
| F42Q N-Term (Mono) | N/A | 97.0 | N/A | N/A | N/A |
| H16N N-term (Biv) | N/A | N/A | N/A | N/A | N/A |
| H16N N-term (Mono) | 1.3 | 0.88 | 0.94 | 1.1 | 1.0 |
| IL15 (Mono) | 3.6 | 1.9 | 0.77 | 1.2 | 0.94 |
| IL2 (Mono) | N/A | N/A | 19.6 | N/A | N/A |
| N88D C-term (Mono) | N/A | 68.1 | N/A | N/A | N/A |
| R38Q N-term (Biv) | N/A | N/A | 6.3 | 37.1 | 23.3 |
| R38Q N-term (Mono) | N/A | N/A | N/A | 79.0 | N/A |
| R38Q/H16N (Biv) | N/A | N/A | 14.8 | 51.9 | 41.4 |
| V91K C-term (Mono) | N/A | N/A | 3.3 | 0.74 | 0.30 |
| WT C-term (Mono) | N/A | 35.0 | 17.4 | 11.3 | 5.5 |
| WT N-term (Biv) | N/A | 97.0 | N/A | N/A | N/A |
| WT N-term (Mono) | N/A | N/A | N/A | N/A | N/A |

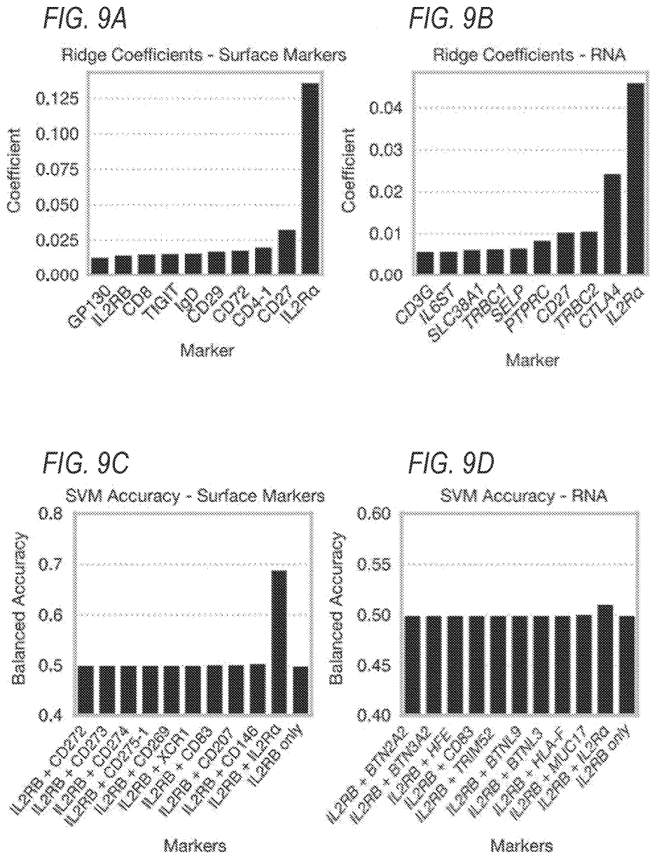

### Example 3. T_{reg}s have Limited Opportunities for Cis Targeting

Whereas IL2Rα is more abundant in T_{reg}s, the difference is subtle compared to that of some off-target cells, making selectively targeted activation more challenging. Consequently, we wondered whether a cis-targeting strategy—in which IL-2 is fused to a domain binding some other T_{reg}-specific surface marker—would provide even greater selectivity. To explore this possibility, we used a CITE-seq data set in which >211,000 human PBMCs were simultaneously analyzed for 228 surface markers coupled with single-cell RNA-seq (GSE164378). Our previous work shows that specificity is conferred by markers expressed at a high ratio between target and off-target cells (FIG. 2, J). As a measure of difference, we calculated the Wasserstein distance and Kullback-Leibler divergence of each surface marker abundance and expression between T_{reg} populations and all off-target PBMCs. These complimentary distance metrics were chosen to reflect two different measures of difference: the Wasserstein distance is maximized when transforming one distribution to another would require changing the cells to a large degree, whereas the Kullback-Leibler distance is maximized when the overlap between two distributions is minimized. We were surprised to find that IL2Rα was the most differentiating and unique marker on T_{reg}s by both proteomic and transcriptomic analysis (FIG. 2, K to N). These results were reinforced by using both a linear and non-linear classifier to identify which surface markers and transcripts were most informative for T_{reg} classification; this analysis again found that IL2Rα was optimal (FIG. 9, A to D). Consequently, binding alternative surface markers would not improve IL-2 T_{reg} selectivity.

### Example 4. Bivalent Fc-Cytokine Fusions have Distinct Cell Specificity but Shared Dynamics

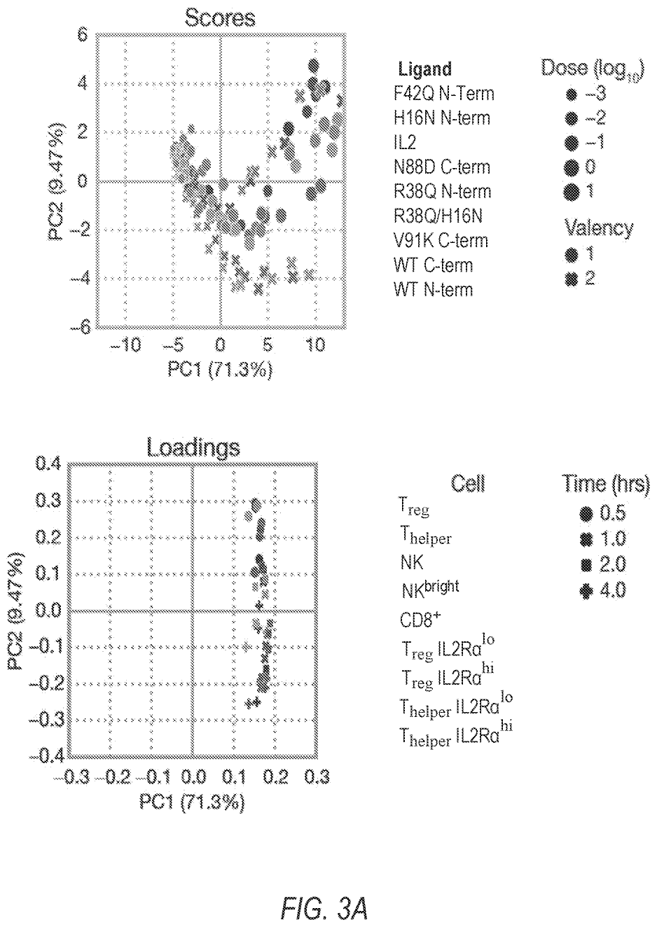

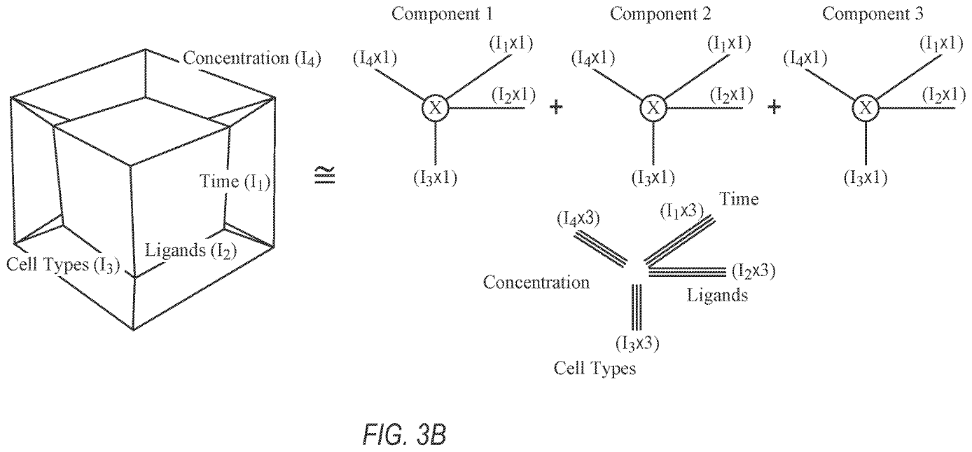

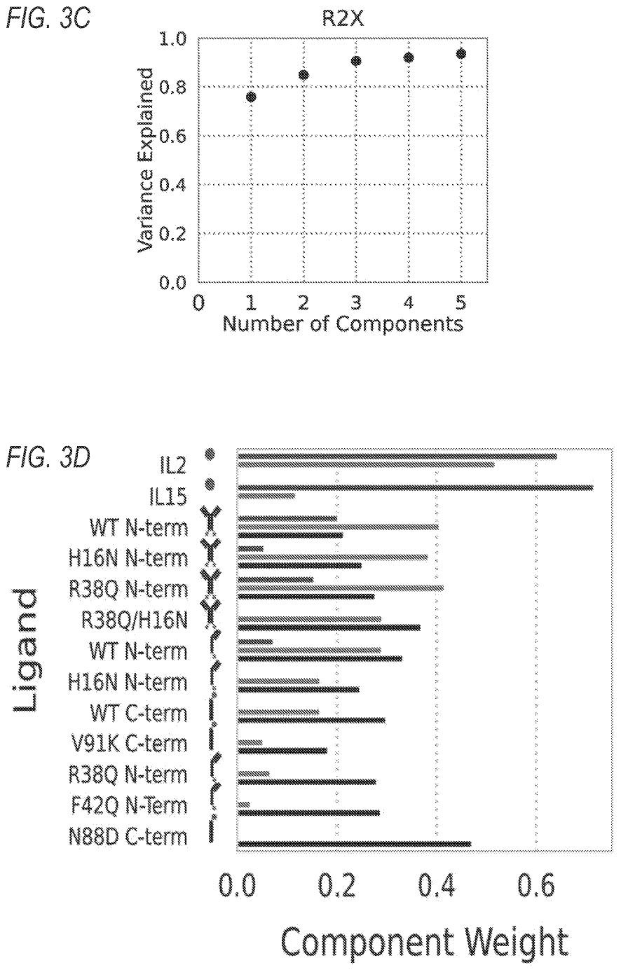

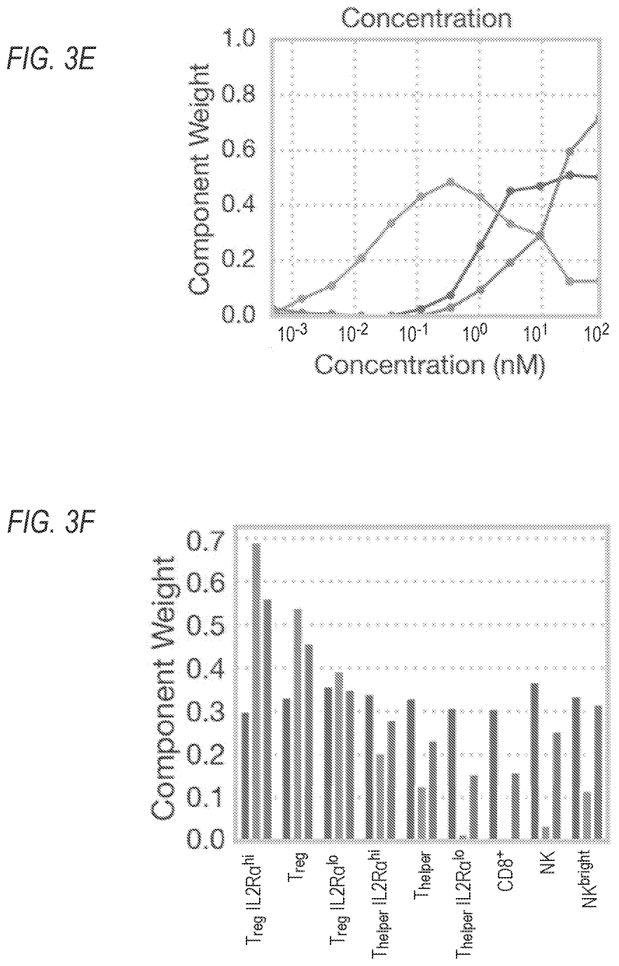

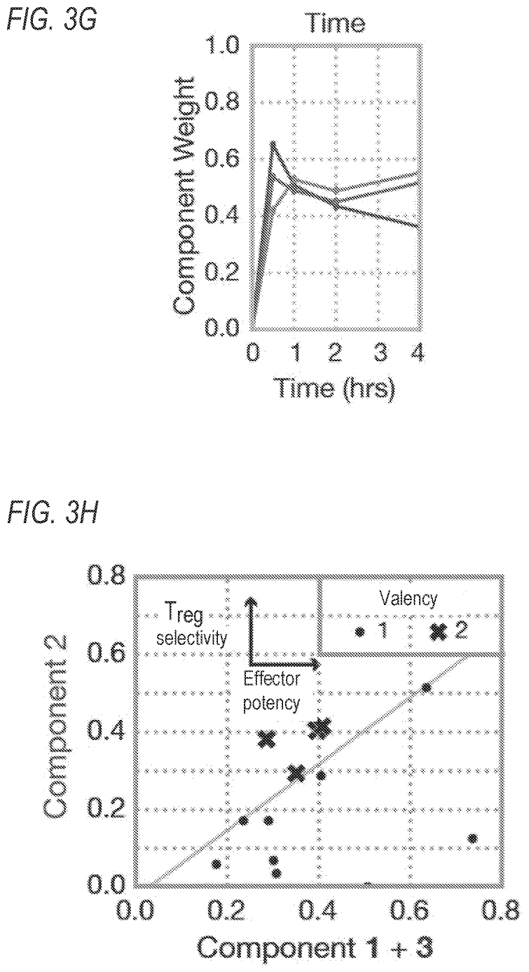

Understanding that selectivity for T_{reg}s must be derived through engineering binding to the IL-2 receptors, we sought to develop a more complete view of the various structural choices for IL-2 fusion design. Exploring variation in response across cell types and ligand treatments is challenging due to its multidimensional nature. Restricting ones' view to a single time point, cell type, or ligand concentration provides only a slice of the picture (FIGS. 1 and 2). Dimensionality reduction is a generally effective tool for exploring multidimensional data. However, flattening our signaling data to two dimensions and using principal components analysis failed to help isolate the effects of concentration, ligand properties, time, and cell type (FIG. 3A). Therefore, to better resolve our data, we organized our profiling experiments into a four-dimensional tensor organized according to the ligand used, concentration, treatment duration, and cell type in the profiling. We then factored this data using non-negative canonical polyadic (CP) decomposition, a technique that represents n-dimensional tensors as additively separable patterns, themselves approximated by the outer product of dimension-specific vectors. We used CP decomposition to derive factors summarizing the influence of each dimension (FIG. 3B). Three components explained roughly 90% of the variance within the dataset (FIG. 3C).

Factorization separated distinct response profiles into separate components, and the effect of each dimension (such as time or concentration) into separate factors. For instance, component 1 almost exclusively represented responses to wild-type cytokines (FIG. 3D), the only ligands which were not Fc-fused, showing a distinct response primarily at high concentrations (FIG. 3E), with broad specificity (FIG. 3F) and a signaling profile peaking at 30 minutes and then more rapidly decreasing (FIG. 3G). An alternative way to interpret the factorization results is to compare profiles within a single factor. For example, component 1 led to a less sustained profile of signaling response as compared to the other signaling patterns (FIG. 3G).

Notably, components 2 and 3 cleanly separated ligands conjugated in bivalent or monovalent forms, respectively (FIGS. 3D and H). In fact, ligand valency was represented more prominently than differences in receptor affinity between muteins. Component 2 had uniquely high T_{reg} specificity (FIG. 3F) most represented at intermediate concentrations (FIG. 3E). Component 2 was also highly correlated with IL2Rα abundance in subsets of T_{reg} and Tₕₑₗₚₑᵣ cells, suggesting that the bivalent molecules' specificity for T_{reg}s is mediated by their higher abundance of IL2Rα. Component 3 had a broader cell response (FIG. 3F) and increased monotonically with concentration (FIG. 3E). Despite these strong differences in specificities, both components had nearly identical time dynamics (FIG. 3G). While other ligand variation influenced the potency and selectivity of each ligand, only the bivalent Fc fusions, regardless of their receptor affinities, more highly weighted the T_{reg}-selective component 2 over components 1 and 3, which represented effector cell response (FIG. 3H). In total, these results indicated that mono- and multivalent cytokines shared identical dynamics and that, although Fc fusion and affinity modulation affect response, ligand valency was a critical and prominent determinant of specificity.

### Example 5. Variation in IL-2 Responses is Explained by a Simple Multivalent Binding Model

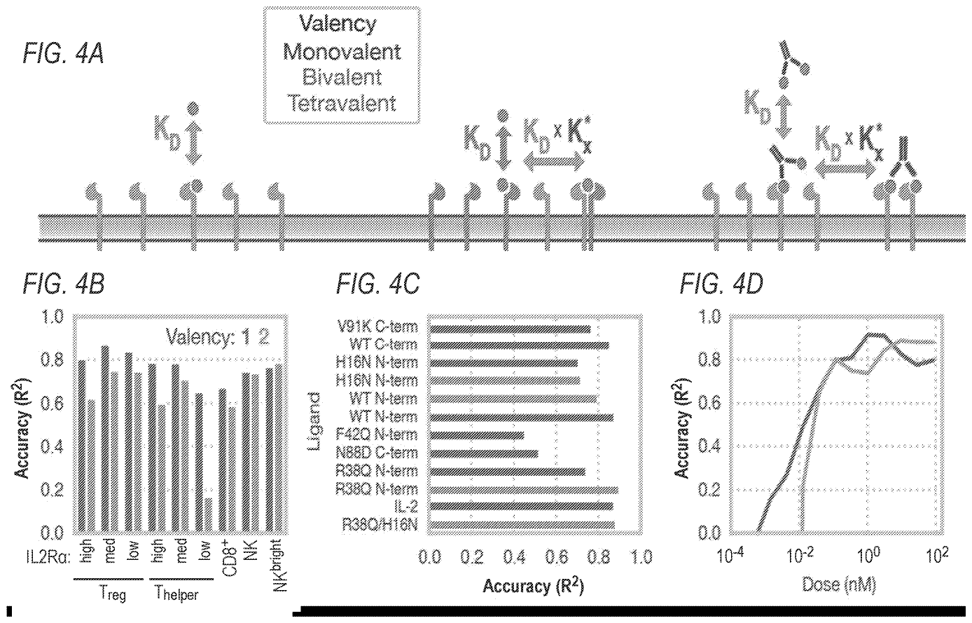

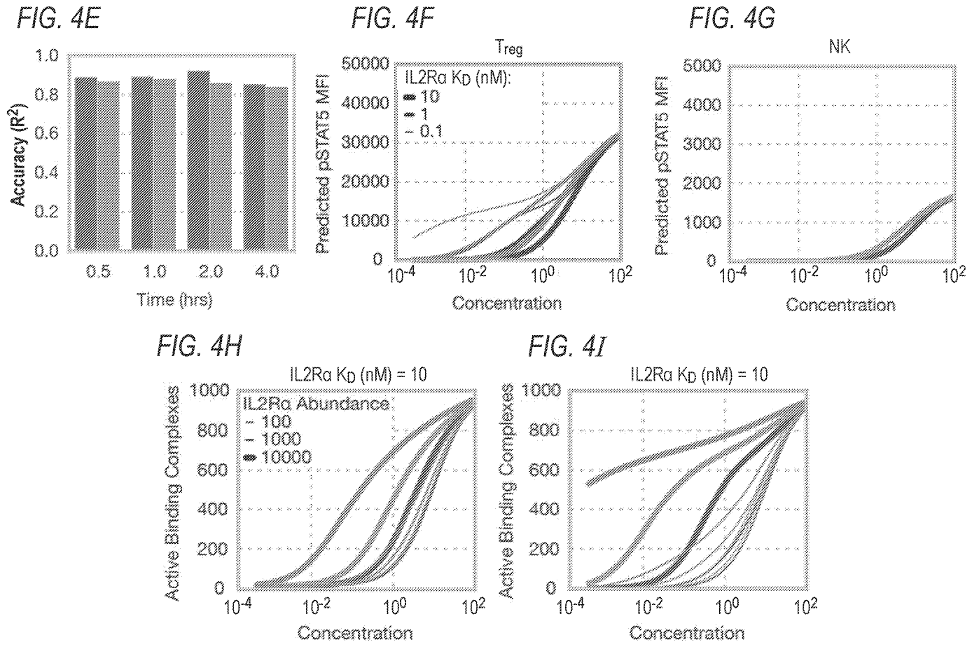

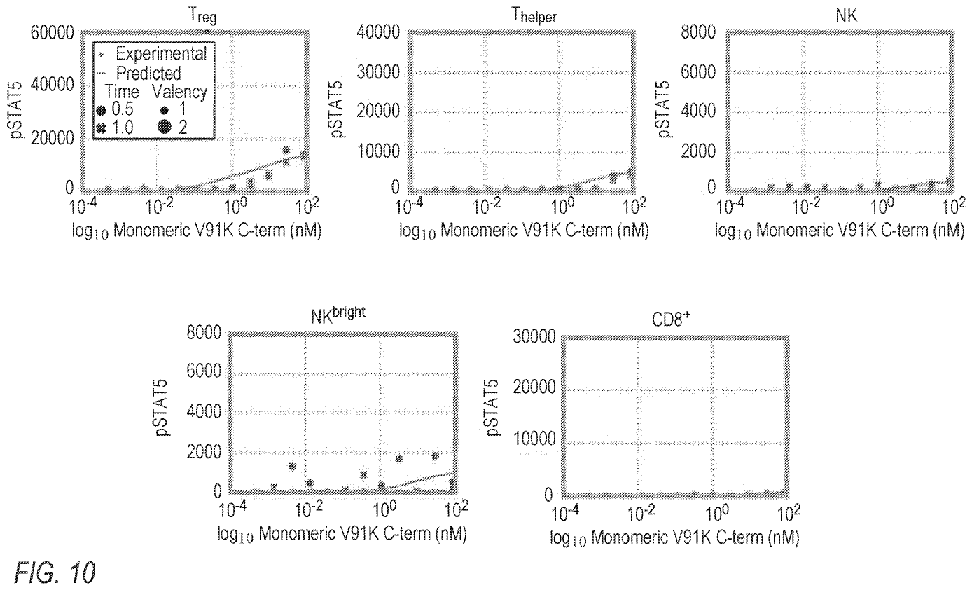

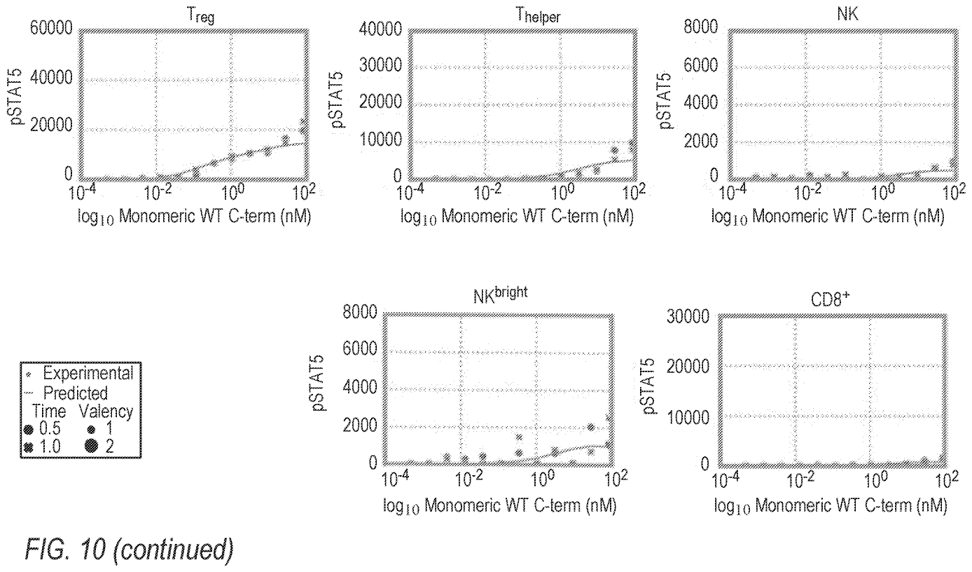

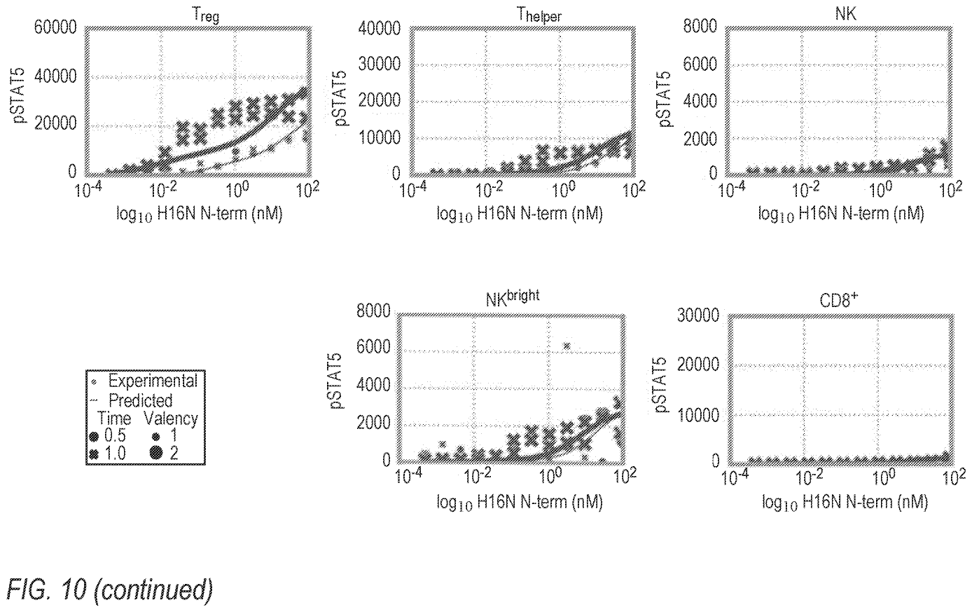

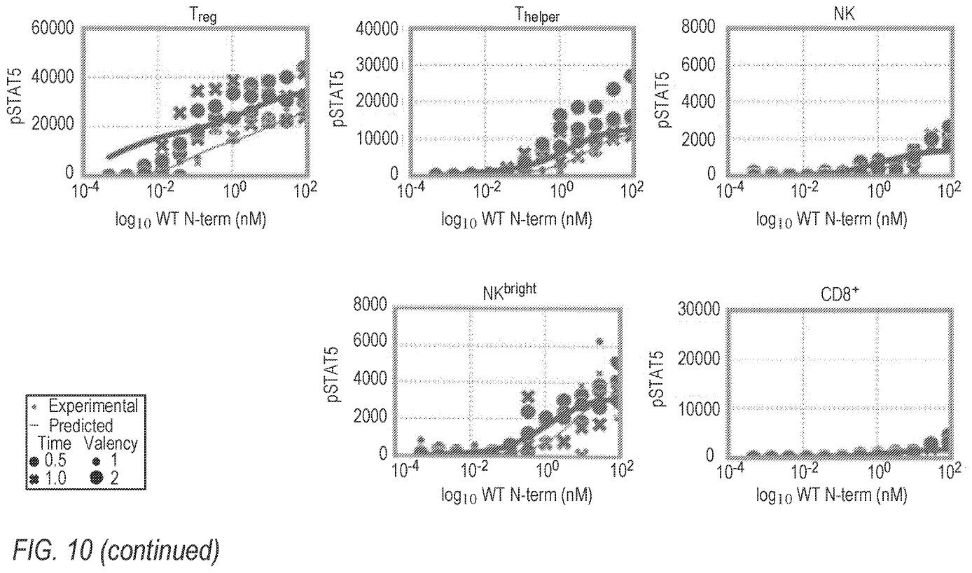

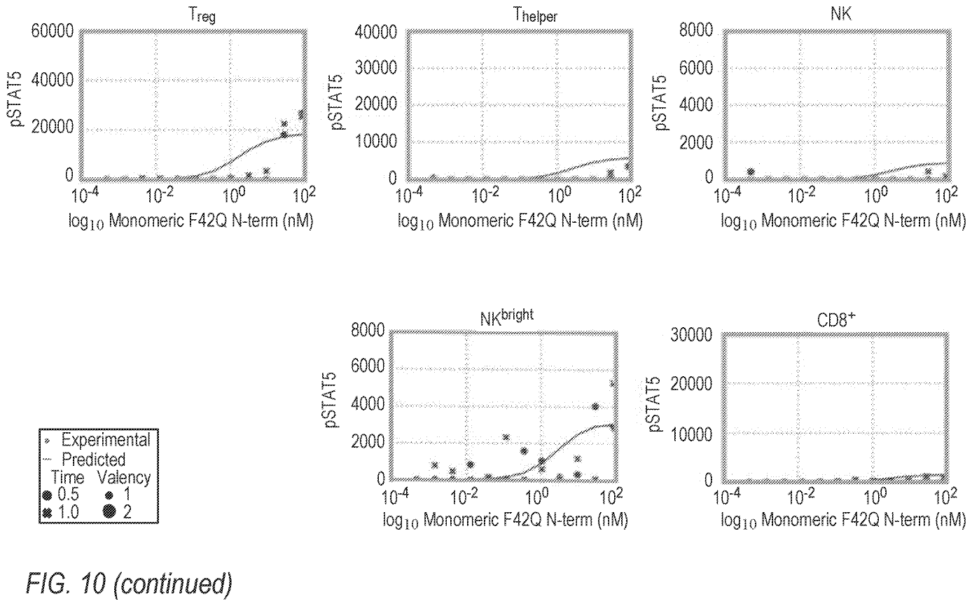

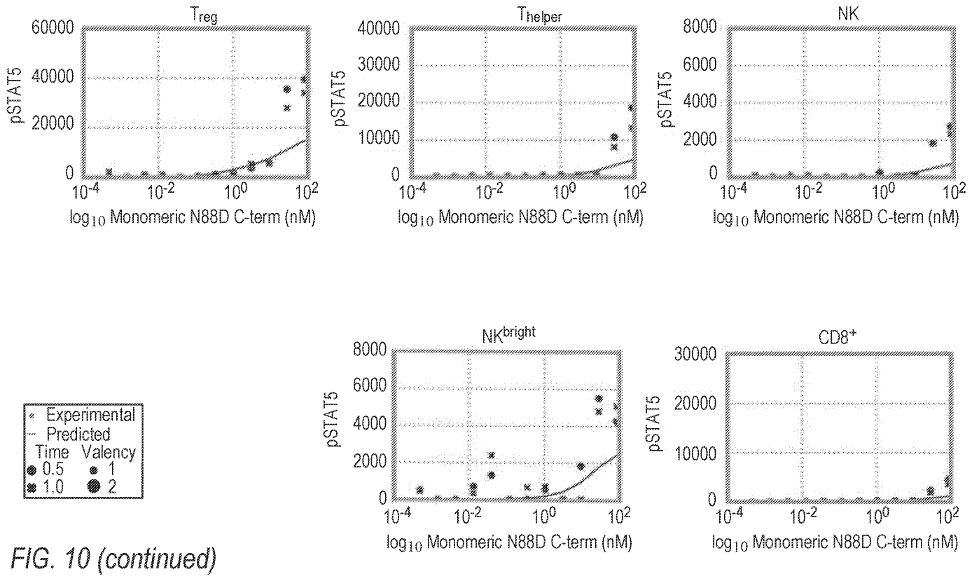

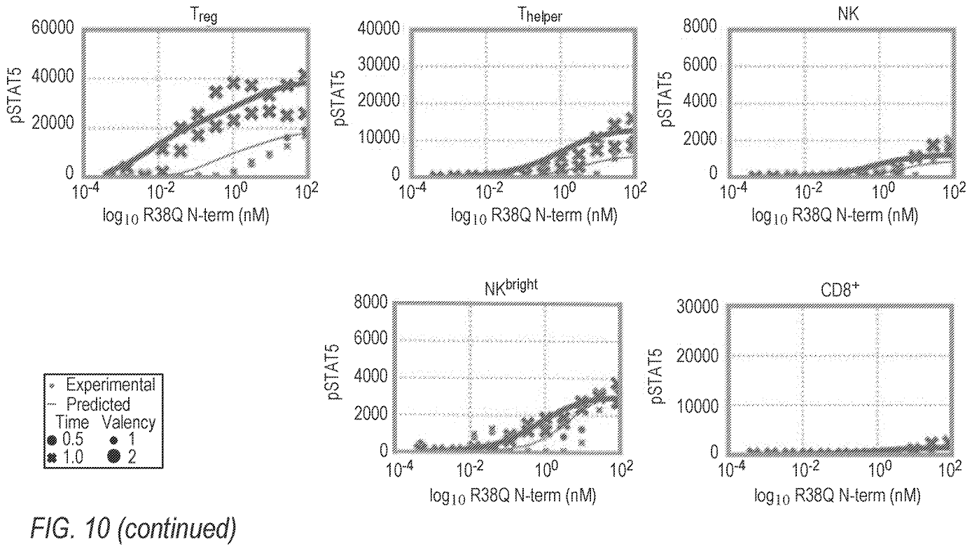

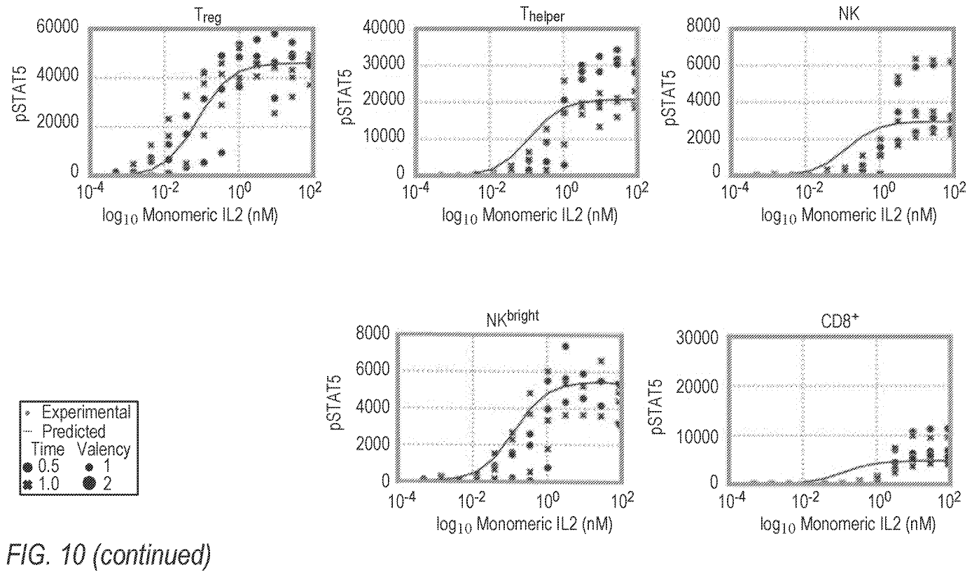

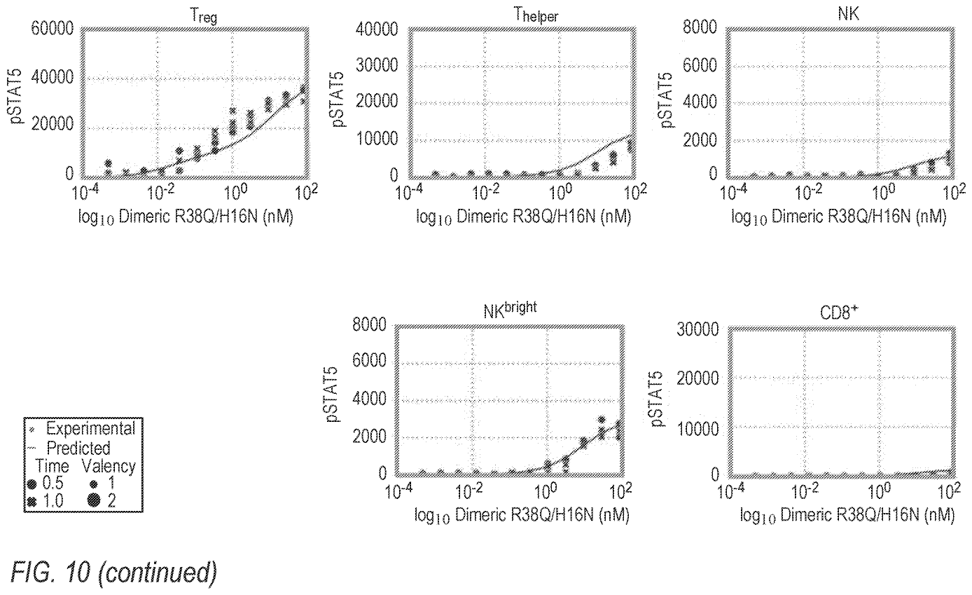

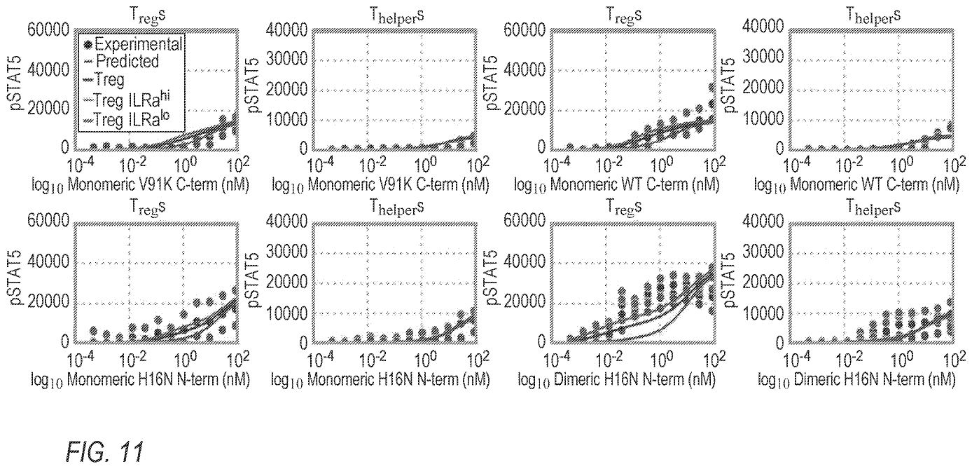

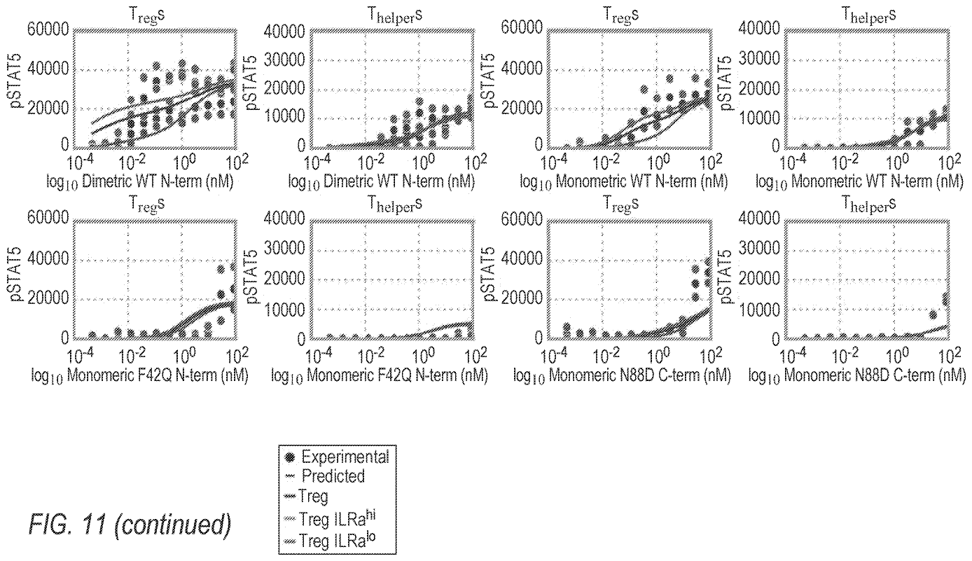

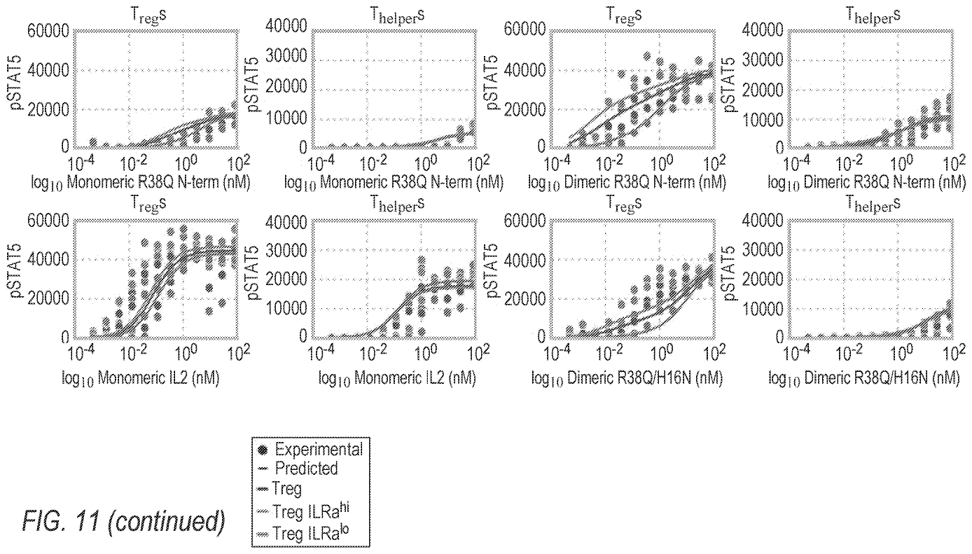

Having observed that T_{reg} selectivity is prominently enhanced by multivalency, we sought to determine whether cell surface binding on its own could explain these selectivity differences. To do so, we applied a two-step, equilibrium, multivalent binding model to predict IL-2 response, assuming that signaling response was proportional to the amount of active receptor-ligand complexes (FIG. 4, A). Within the model, ligand binding first occurs with kinetics equivalent to the single binding site, and then subsequent interactions occur proportionally to affinity, adjusted by K*, a cross-lining constant that corrects for differences between monovalent and multivalent interactions. We fit this model to our signaling experiments and evaluated its concordance with the data. The model is very simple, with the cross-linking parameter being the only non-scaling fit parameter; this parameter had an optimum at 1.2×10⁻¹¹ #/cell, consistent with that seen for other receptor families. Overall, we observed remarkable consistency between predicted and observed responses (R²=0.85; FIG. 10), and accuracy was maintained when examining data subsets, including individual cell types and ligands (FIGS. 4, B and C).

To ensure that our model was not simply capturing a trend towards higher signaling with increasing concentration, we examined our model's accuracy within each concentration (FIG. 4D). Our model did not predict response at the lowest concentrations as there was little to no response in the data itself but increased in accuracy at concentrations where responses were observed. Finally, we examined how the model's accuracy varied within each timepoint (FIG. 4E); each was predicted with consistent accuracy. Some decrease in model accuracy would be expected, given that longer treatments likely involve various compensatory mechanisms such as the degradation or increased transcription of IL-2 receptor subunits. In total, multivalent cell surface binding showed quantitative agreement with the pattern of cell-type-specific responses to IL-2 muteins, supporting that the specificity enhancement of bivalency is derived from receptor avidity effects and is explained by a simple model of cell surface binding.

Upon finding that our model was broadly predictive of cell-type specific signaling responses, we sought to use our model to understand and visualize how valency and affinity interact to determine T_{reg} selectivity. Here, our model showed that T_{reg} response is strongly governed by IL2Rα affinities and that these effects have an exceptionally strong relationship with valency, particularly at intermediate cytokine doses, while NK signaling barely varied across ligands of varying affinities (FIGS. 4, F and G). We then used the model to explore how receptor abundance affects multivalent ligand binding (FIGS. 4, H and I). Here, theoretical cell populations expressing 10⁴ IL2Rα and 10³ IL2Rβ molecules varied widely in their response to multivalent IL-2 muteins (FIG. 4H), while cells expressing very few IL2Rα receptors and the same abundance of IL2Rβ barely varied in their response (FIG. 4I). Therefore, we concluded that multivalent cytokines with high IL2Rα affinities uniquely and selectively target T_{reg}s through IL2Rα-mediated avidity effects.

### Example 6. Multivalency Provides a General Strategy for Enhanced Signaling Selectivity and Guides the Development of Superior IL-2 Muteins

Given that a simple binding model accurately predicted cell type-specific responses to IL-2 and that bivalent, Fc-fused IL-2 muteins have favorable specificity properties, we computationally explored to what extent multivalency might be a generally useful strategy. While monovalent ligand binding scales linearly with receptor abundance, multivalent ligands bind nonlinearly depending upon receptor abundance. Thus, multivalent ligands should be able to selectively target cells with uniquely high expression of certain γ_{c} family receptors.

Valency enhancements are only apparent with coordinated changes in receptor-ligand binding affinities. Therefore, we optimized the receptor affinities of simulated ligands while varying valency. We first designed IL-2 muteins of varying valency to obtain optimal T_{reg} specificity (FIG. 5A). As expected, ligand valency increased achievable selectivity past that possible using a monovalent cytokine format at any receptor affinities. Muteins of higher valency required reduced IL2Rα affinity to achieve optimal T_{reg} selectivity (FIG. 5B). We then explored whether IL-2 muteins lacking IL2Rα binding could selectively target NK cells, based on their uniquely high expression of IL2Rβ, with similar results; IL-2 muteins of higher valency were predicted to be increasingly selective for activation of NK cells, so long as IL2Rβ/γ_{c} affinity was coordinately decreased (FIGS. 5, C and D). Finally, we explored whether multivalent IL-7 could be used to target Tₕₑₗₚₑᵣs, as they express high amounts of IL7Rα (FIG. 7I). We again found that ligands of higher valency should achieve higher selectivity for these cells, but that the benefits of valency were less than the targeting of Trees or NK cells using IL-2 mutants because CD8⁺ T cells have similar IL7Rα amounts (FIG. 5E). These benefits were again contingent on decreasing IL7Rα affinity at higher valency (FIG. 5F).

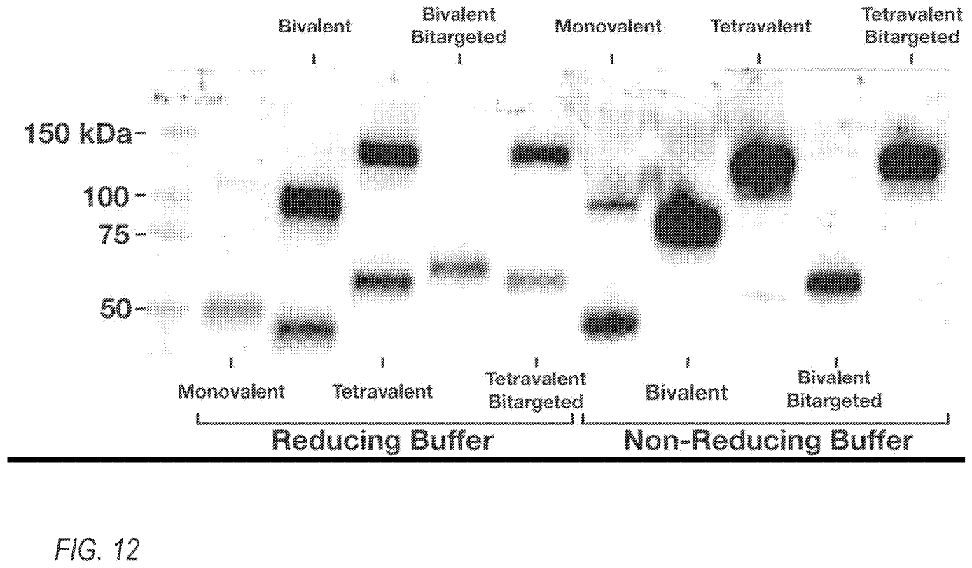

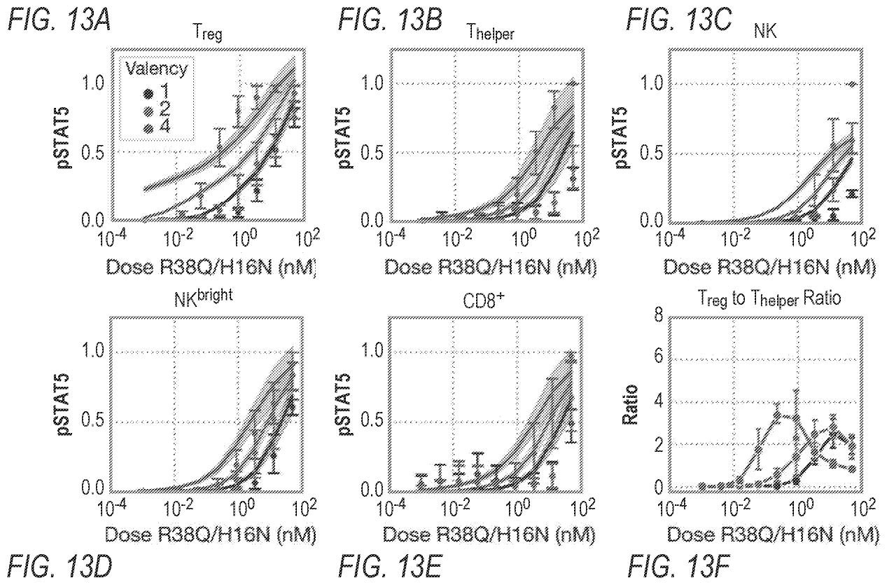

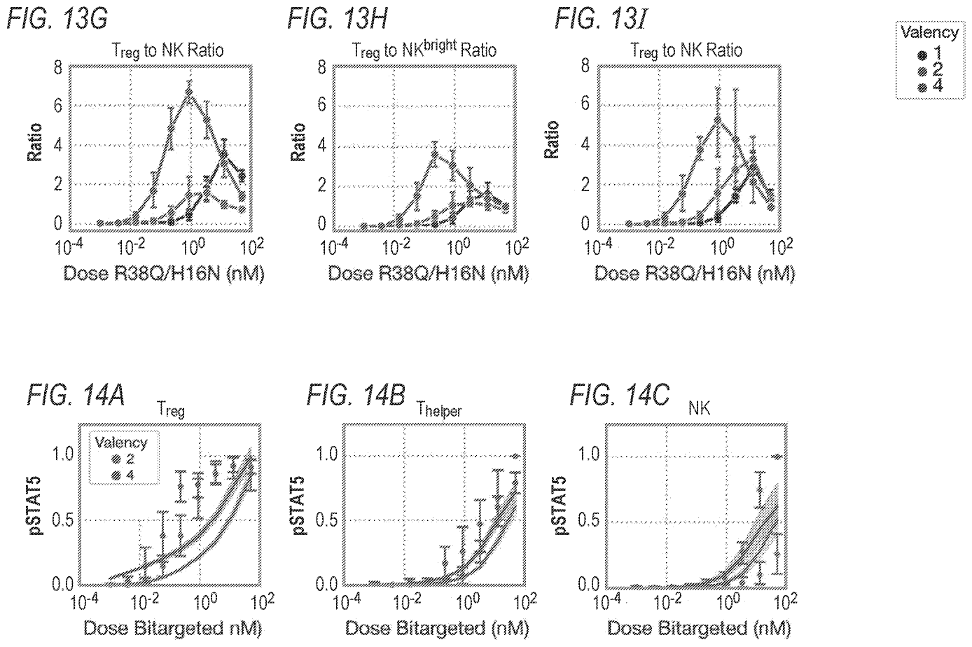

To experimentally show that muteins of higher valency could be engineered to increase T_{reg} selectivity, we expressed and purified Fc fusions of R38Q/H16N IL-2 in monovalent, bivalent, and tetravalent formats (FIG. 12). PBMCs from five donors were used to account for patient-to-patient variability. Tetravalent IL-2 was designed by Fc-fusing IL-2 muteins at both the C- and N-terminus and allowing the Fc to dimerize (FIG. 5G). The harvested cells were stimulated for 30 minutes and stained for cell type markers as well as pSTAT5. R38Q/H16N was selected as the mutant closest to optimal binding affinities in tetravalent form, though further optimization is possible (FIGS. 1B and 5B). As predicted, valency increased the responsiveness of both T_{reg}s and off-target immune cells at each concentration (FIG. 13, A to E). However, the T_{reg} response increase far exceeded that in off-target cells; consequently, tetravalent R38Q/H16N was able to achieve much greater T_{reg} selectivity than bivalent and monovalent formats—two IL-2 fusions with near-optimal selectivity in our initial panel (FIG. 5, H to K).

In total, these results show that valency beyond bivalency has unexplored potential for engineered cytokines with enhanced therapeutic potency and reduced toxicity. Critically, our tetravalent R38Q/H16N far outperformed its lower valency counterparts that already represented state-of-the-art selectivity. These results demonstrate that multivalent complexes can achieve selective cytokine signaling in T_{reg}s beyond what is achievable with only changes to receptor affinity. They also show the benefit of mechanistic modeling to guide ligand design, particularly when ligand affinity must be considered together with other parameters such as valency.

### Example 7. Bitargeted IL-2-Fc Fusions Demonstrate Even Greater T_{reg} Selectivity

Through the CITE-seq data analysis, we found that IL2Rα was the optimal surface target for T_{reg} selectivity (FIG. 2, I to M). This result was further strengthened when we integrated these data with our binding model and ligand optimization approach. We used the model to consider whether WT IL-2 fused to a selective binder for any surface markers could increase T_{reg} selectivity. WT IL-2 fusion to an IL2Rα binder was predicted to enhance T_{reg} selectivity over off-target immune cells (FIGS. 6, A and B). This was initially surprising because IL-2 itself binds IL2Rα, but indicated to us that multivalent complexes provide the potential opportunity to decouple T_{reg} selectivity (by binding IL2Rα) from cytokine potency.

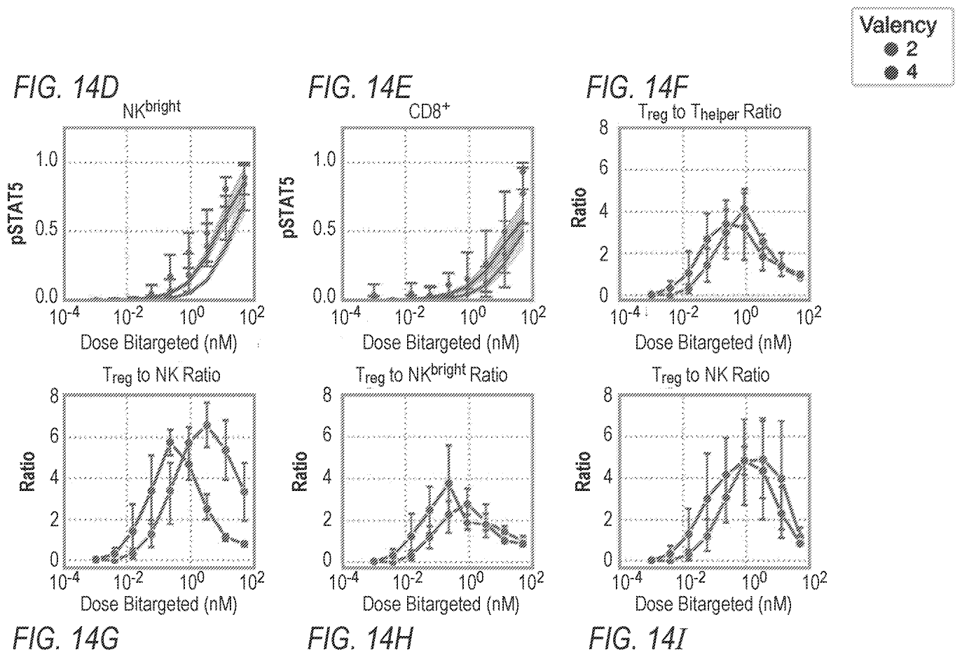

To decouple cell-selective binding from signaling response, we expressed an asymmetric Fc fusion including both a signaling-competent R38Q/H16N IL-2 and signaling-deficient V91K/D20A/M104V IL-2 with only IL2Rα binding (FIG. 6C and FIG. 12). V91K/D20A/M104V IL-2 is reported to effectively eliminate IL2Rβ binding while maintaining IL2Rα interaction. We henceforth refer to this asymmetric construct as “bitargeted” IL-2 to reflect the inclusion of two IL-2 muteins with separate signaling and targeting roles. To ensure homogeneity of signaling and non-signaling IL-2, bivalent bitargeted ligand was designed by introducing Fc mutations preventing Fc dimerization (FIG. 6C). Tetravalent bitargeted constructs were predicted to have greater T_{reg} specificity than their non-bitargeted counterparts for any IL2Rβ affinity, using either the IL2Rα affinity of WT or R38Q/H16N (FIG. 6D). We tested our bivalent and tetravalent bitargeted constructs by again stimulating PBMCs gathered from five donors and quantifying their pSTAT5 responses. Both bivalent and tetravalent bitargeted ligand increased or maintained high potency in T_{reg} cells (FIG. 14, A to E). This potency translated to greater T_{reg} selectivity for both bivalent and tetravalent bitargeted forms, both of which outperformed any previously characterized monovalent and bivalent IL-2 fusions, and modestly outperformed tetravalent R38Q/H16N (FIG. 6, E to H).

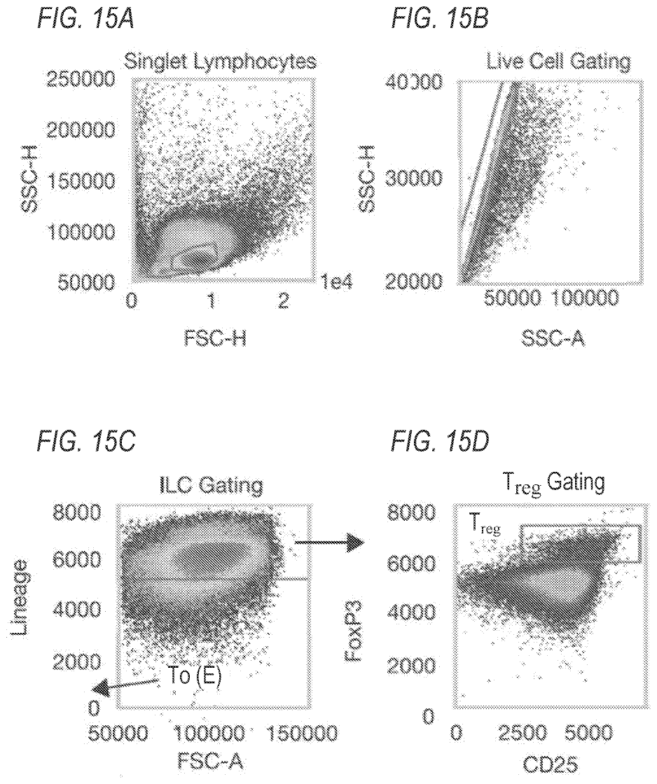

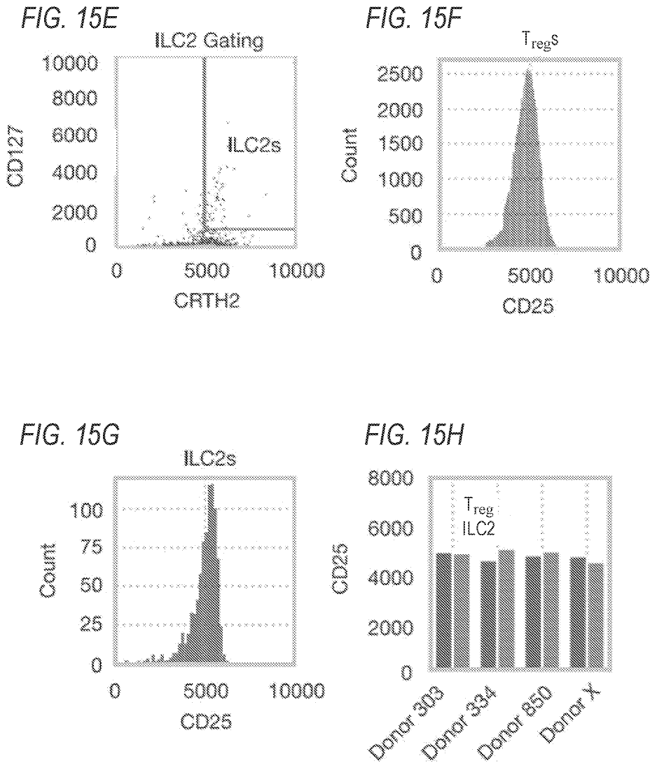

Though both our tetravalent R38Q/H16N molecules and bitargeted constructs were able to target T_{reg}s with superior selectivity with respect to Tₕₑₗₚₑᵣ, NK, NK^{bright}, and CD8⁺ cells, principally through the targeting of IL2Rα, we considered whether our molecule could also select for T_{reg}s with respect to type 2 innate lymphoid cells (ILC2s). ILC2s are able to secrete large quantities of inflammatory cytokines such as IL-5 and IL-13, are known to respond to IL-2 through expression of IL2Rα, and have been implicated as a potential source of eosinophilia in low-dose IL-2 therapy. We quantified the abundance of IL2Rα on ILC2s and T_{reg}s from four donors and found that both populations expressed nearly identical levels of the receptor (FIG. 15H). Thus, neither our nor any other existing approaches for targeting T_{reg}s through the IL-2 receptors themselves offer superior selectivity against ILC2s.

We again applied non-negative CP decomposition of the R38Q/H16N and bitargeted ligand signaling responses to summarize our ligand engineering efforts (FIG. 6, I to L). Three components captured >90% of the variation in the data. Here, T_{reg} responses were primarily represented by component 2, Teff cell responses by component 1, and NK cell responses by component 3 (FIG. 6J). Valency again determined ligand component 2 weight most potently, with both tetravalent constructs, bitargeted or R38Q/H16N, demonstrating highest component 2 weight. However, while the bivalent bitargeted construct demonstrated no NK cell activity (component 3), both tetravalent constructs had some NK activity (FIG. 6K). Thus, our experimental results demonstrate that tradeoffs still exist between T_{reg} potency and selectivity. As before, T_{reg} selectivity was maximized at low and intermediate dosages (FIG. 6L). Despite these tradeoffs, the selectivity demonstrated by both model-guided “cis”-targeting or higher valency fusion formats greatly improved upon the selectivity possible with existing approaches.

### Example 8. Preparing Multispecific Multivalent Cytokines

Various forms of multispecific multivalent cytokines may be prepared using fusions of a signaling-competent cytokine such as wild type IL-2 or IL-4 and a signaling-deficient cytokine such as an IL-2 mutein with V91K/D20A/M104V. In some embodiments, an IL-2 that is signal competent, such as R38Q and/or H16N, may be used, having decreased IL2Rβ affinity or increased selectivity for T_{reg}s. In some embodiments, a single-chain polypeptide is expressed having at least one signaling-competent cytokine such as wild type IL-2 or IL-4, and a signaling-deficient cytokine such as an IL-2 mutein with V91K/D20A/M104V. In some embodiments, a linker such as (G₄S)₄ may be provided between the cytokine sequences.

In some embodiments, a single-chain polypeptide such as mentioned above are expressed with a cross-linkable (dimerizable) or non-cross-linkable (monovalent) Fc domain, such as a Fc CH2/CH3 hinge region, with or without a C-terminal lysine. Such dimerizing Fc allows for the disulfide crosslinking of two Fc regions to form a dimer of the aforementioned single polypeptide chains, which if each has at least two cytokines (at signaling competent and signaling deficient), forms a tetravalent, multispecific biomolecule. In another embodiment, a tandem sequence of two cytokines at the N terminus and one at the C terminus (at least one signaling competent and at least one signaling deficient) forms a trivalent Fc fusion polypeptide, which if synthesized with a dimerizable Fc region, when dimerized forms a multispecific hexavalent cytokine. In another embodiment, a tandem sequence of two cytokines expressed as a fusion polypeptide at both the N- and C-termini of Fc forms a tetravalent single-chain polypeptide (with at least one signaling competent cytokine and at least one signaling deficient cytokine), which if dimerized forms an multispecific octavalent cytokine. Variations in the design and preparation of such multispecific multivalent cytokines are fully embraced herein.

### Example 9. Treatment Regimens

A patient presenting with the autoimmune disease systemic lupus erythematosus is started on a regimen of a tetrameric, multispecific multivalent IL-2 described herein comprising dimerized SEQ ID NO:07, administered once daily by intravenous infusion, using a dose level that is identified in a clinical trial to achieve T_{reg} expansion without expanding other, undesirable T cell populations. The patient's T_{reg} abundance increases over time, and resolution of the disease is observed.

A patient to receive an organ transplant from a donor is identified by HLA matching or diagnostic signs of rejection to be a candidate for T_{reg} enhancement therapy using a tetravalent multispecific multivalent cytokine. The patient is administered lipid nanoparticles comprising SEQ ID NO:35, which is taken up by cells in the patient and produces a tetravalent bitargeted multispecific multivalent cytokine by cross-linking of the Fc regions. The patient's T_{reg} abundance increases over time, and organ rejection is suppressed.

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

All references cited herein are incorporated herein by reference in their entireties.

What is claimed is:

1. A multispecific multivalent biomolecule comprising two or more covalently linked cytokines or variants thereof, wherein the biomolecule comprises at least a first cytokine or variant thereof, and at least a second cytokine or variant thereof, wherein:
a. the first cytokine or variant thereof is a signaling-competent cytokine or a signaling-competent variant thereof; and
b. the second cytokine or variant thereof is a same or different cytokine from the first cytokine, and is signaling-deficient compared to that of a native second cytokine.

2. The multispecific multivalent biomolecule of claim 1 wherein the signaling-deficient cytokine has increased receptor affinity, decreased receptor affinity, increased receptor signaling, decreased receptor signaling, or any combination thereof.

3. The multispecific multivalent biomolecule of claim 2 wherein decreased receptor signaling is substantially no receptor signaling.

4. The multispecific multivalent biomolecule of any one of claims 1-3, wherein the first cytokine or variant thereof and the second cytokine of variant thereof are the same cytokine.

5. The multispecific multivalent biomolecule of any one of claims 1-3, wherein the first cytokine or variant thereof and the second cytokine or variant thereof are different cytokines.

6. The multispecific multivalent biomolecule of any one of claims 1-5, comprising at least 2, at least 3, at least 4, at least 5, at least 6, at least 7, or at least 8 cytokines or variants thereof.

7. The multivalent biomolecule of claim 6, comprising 2, 3, 4, 5, 6, 7 or 8 cytokines or variants thereof.

8. The multivalent biomolecule of any one of claims 1-7, wherein the first cytokine or variant thereof and the second cytokine or variant thereof are covalently linked by being present on a fusion polypeptide.

9. The multivalent biomolecule of any one of claims 1-7, wherein the first cytokine or variant thereof and the second cytokine or variant thereof cytokines are covalently linked by cross-linking.

10. The multivalent biomolecule of any one of claims 1-7, wherein the cytokines are covalently linked by a first cytokine or variant thereof being present on a fusion polypeptide and cross-linked to another fusion polypeptide comprising the second cytokine or variant thereof.

11. The multivalent biomolecule of claim 10, wherein the cytokines are covalently linked by cross-linking a fusion polypeptide comprising a first cytokine or variant thereof and the second cytokine or variant thereof to at least another cytokine.

12. The multispecific multivalent biomolecule of any one of claims 1-11, wherein the cytokines or variants thereof are expressed as Fc fusion proteins of the cytokines or variants thereof with human IgG1 Fc.

13. The multispecific multivalent biomolecule of claim 12, wherein the fusion protein comprises the cytokines or variants thereof fused to the N- or C-terminus of human IgG1 Fc.

14. The multispecific multivalent biomolecule of claim 13, wherein the cytokines or variants thereof are fused to the N- or C-terminus of human IgG1 Fc through a (G₄S)₄ linker.

15. The multispecific multivalent biomolecule of any one of claims 1-14, wherein at least the first cytokine or the second cytokine is a lymphokine, an interferon, an interleukin, a chemokine or tumor necrosis factor.

16. The multispecific multivalent biomolecule claim 15, wherein at least the first cytokine or the second cytokine is a common γ-chain receptor cytokine.

17. The multispecific multivalent biomolecule claim 15, wherein both the first cytokine and the second cytokine are a common γ-chain receptor cytokine.

18. The multispecific multivalent biomolecule of claim 17, wherein the common γ-chain receptor cytokine is independently one or more of IL-2, IL-4, IL-7, IL-9, IL-15 or IL-21, or a variant thereof.

19. The multispecific multivalent biomolecule of any one of claims 1-18 comprising two or more covalently linked cytokines or variants thereof, wherein the biomolecule comprises at least a first cytokine or variant thereof, and at least a second cytokine or variant thereof, wherein:
a. the first cytokine or variant thereof is a signaling-competent common γ-chain receptor cytokine or a signaling-competent variant thereof; and
b. the second cytokine or variant thereof is a same or different common γ-chain receptor cytokine from the first cytokine, and is signaling-deficient compared to that of a native second cytokine.

20. The multispecific multivalent biomolecule of any one of claims 1-19 wherein at least one common γ-chain receptor cytokine, or variant thereof, comprises a signal sequence.

21. The multispecific multivalent biomolecule of any one of claims 1-19 wherein the signaling-deficient cytokine has at least one mutation.

22. The multispecific multivalent biomolecule of claim 21 wherein the at least one mutation is an inactivating mutation.

23. The multispecific multivalent biomolecule of any one of claims 19-22 comprising a signaling-competent IL-2 and a signaling-deficient IL-2.

24. The multispecific multivalent biomolecule of claim 23 wherein the signaling-competent IL-2 is native IL-2 or IL-2 having a R38Q and/or H16N mutation.

25. The multispecific multivalent biomolecule of claim 23 wherein the signaling-deficient IL-2 has a V91K, D20A or M104V mutation, or any combination thereof.

26. The multispecific multivalent biomolecule of claim 19 comprising two signaling-competent IL-2 and two signaling-deficient IL-2 muteins.

27. The multispecific multivalent biomolecule of any one of claims 19-22 selected from SEQ ID NOs: 07, 08, 09, 11, 13, 15, 17, 19, 20, 22, 24, 26, 29, 31, 33, 35 and 37.

28. The multispecific multivalent biomolecule of any one of claims 19-22 comprising a signaling-competent IL-4 and a signaling-deficient IL-4.

29. The multispecific multivalent biomolecule of claim 28 wherein the signaling-competent IL-4 is native IL-4 or IL-4 having R121D/Y124D mutation.

30. The multispecific multivalent biomolecule of claim 28 wherein the signaling-deficient IL-4 has a R121D/Y124D mutation, or any combination thereof.

31. The multispecific multivalent biomolecule of claim 19 comprising two signaling-competent IL-4 and two signaling-deficient IL-4 muteins.

32. The multispecific multivalent biomolecule of any one of claims 19-22 comprising a signaling-competent IL-7 and a signaling-deficient IL-2 mutein.

33. The multispecific multivalent biomolecule of claim 32 wherein the signaling-competent IL-7 is native IL-7.

34. The multispecific multivalent biomolecule of claim 32 wherein the signaling-deficient IL-2 has a V91K, D20A or M104V mutation, or any combination thereof.

35. The multispecific multivalent biomolecule of claim 32 comprising two signaling-competent IL-7 and two signaling-deficient IL-2 muteins.

36. The multispecific multivalent biomolecule of any one of claims 19-22 comprising a signaling-competent IL-9 and a signaling-deficient IL-2 mutein.

37. The multispecific multivalent biomolecule of claim 36 wherein the signaling-competent IL-9 is native IL-9.

38. The multispecific multivalent biomolecule of claim 36 wherein the signaling-deficient IL-2 has a V91K, D20A or M104V mutation, or any combination thereof.

39. The multispecific multivalent biomolecule of claim 36 comprising two signaling-competent IL-9 and two signaling-deficient IL-2 muteins.

40. The multispecific multivalent biomolecule of any one of claims 19-22 comprising signaling-competent IL-15 and a signaling-deficient IL-2 mutein.

41. The multispecific multivalent biomolecule of claim 40 wherein the signaling-competent IL-9 is native IL-15.

42. The multispecific multivalent biomolecule of claim 40 wherein the signaling-deficient IL-2 has a V91K, D20A or M104V mutation, or any combination thereof.

43. The multispecific multivalent biomolecule of claim 40 comprising two signaling-competent IL-15 and two signaling-deficient IL-2 muteins.

44. The multispecific multivalent biomolecule of any one of claims 19-22 comprising signaling-competent IL-21 and a signaling-deficient IL-2 mutein.

45. The multispecific multivalent biomolecule of claim 44 wherein the signaling-competent IL-9 is native IL-21.

46. The multispecific multivalent biomolecule of claim 44 wherein the signaling-deficient IL-2 has a V91K, D20A or M104V mutation, or any combination thereof.

47. The multispecific multivalent biomolecule of claim 44 comprising two signaling-competent IL-21 and two signaling-deficient IL-2 muteins.

48. The multispecific multivalent biomolecule of any one of claims 19-22 comprising signaling-competent IL-9 and a signaling-deficient IL-2 mutein.

49. The multispecific multivalent biomolecule of claim 48 wherein the signaling-competent IL-9 is native IL-9.

50. The multispecific multivalent biomolecule of claim 48 wherein the signaling-deficient IL-2 has a V91K, D20A or M104V mutation, or any combination thereof.

51. The multispecific multivalent biomolecule of claim 48 comprising two signaling-competent IL-9 and two signaling-deficient IL-2 muteins.

52. The multispecific multivalent biomolecule of any one of claims 1-52, wherein the biomolecule comprises a dimer of two cross-linked SEQ ID NO:7, two cross-linked SEQ ID NO:20, a cross-linked SEQ ID NO:07 and SEQ ID NO:20, two cross-linked SEQ ID NO:22, two cross-linked SEQ ID NO:24, two cross-linked SEQ ID NO:29, two cross-linked SEQ ID NO:31, two cross-linked SEQ ID NO:35, a cross-linked SEQ ID NO: 29 and SEQ ID NO:20, a cross-linked SEQ ID NO:7 and SEQ ID NO:35, a cross-linked SEQ ID NO:31 and SEQ ID NO:7, a cross-linked SEQ ID NO:31 and SEQ ID NO: 20, a cross-linked SEQ ID NO:31 and SEQ ID NO:22, a cross-linked SEQ ID NO: 31 and SEQ ID NO:24, or a cross-linked SEQ ID NO:31 and SEQ ID NO:29.

53. The multispecific multivalent biomolecule of any one of claim 1-51, wherein the biomolecule has enhanced selectivity for driving T_{reg}-mediated immune suppression compared to a native cytokine.

54. A method for modulating the immune system of a subject, comprising administering to a subject in need thereof the multispecific multivalent biomolecule or cells expressing the multispecific multivalent biomolecule of any one of claims 1-51, or administering a nucleic acid molecule encoding said multispecific multivalent biomolecule or a component thereof.

55. The method of claim 54, wherein the multispecific multivalent biomolecule is used for treating cancer.

56. The method of claim 54, wherein the modulating is suppressing immune responses in the subject.

57. The method of claim 54, wherein the multispecific multivalent biomolecule is used for treating an autoimmune disease or preventing, mitigating or reducing transplant rejection.

58. The method of claim 57, wherein the autoimmune disease is systemic lupus erythematosus.

59. A multispecific multivalent cytokine comprising any one of SEQ ID NOs: 07, 08, 09, 11, 13, 15, 17, 19, 20, 22, 24, 26, 29, 31, 33, 35 and 37.

60. A multispecific multivalent cytokine comprising a cross-linked dimer of any one of SEQ ID NOs: 07, 20, 22, 24, 29, 31, 35 or any homodimeric or heterodimeric combination thereof.

61. A pharmaceutical composition comprising a multivalent cytokine of any one of claims 1-60.

62. A pharmaceutical composition of claim 61 comprising SEQ ID NOs: 07, 08, 09, 11, 13, 15, 17, 19, 20, 22, 24, 26, 29, 31, 33, 35 or 37, or a cross-linked dimer of any one of SEQ ID NOs: 07, 20, 22, 24, 29, 31 or 35, or any combination thereof.

63. A nucleic acid molecule encoding a multispecific multivalent cytokine or component thereof of any one of claims 1-60.

64. A nucleic acid molecule encoding any one of SEQ ID NOs: 07, 08, 09, 11, 13, 15, 17, 19, 20, 22, 24, 26, 29, 31, 33, 35 and 37.

65. A vector comprising a nucleic acid molecule encoding a multispecific multivalent cytokine or component thereof of any one of claims 1-60.

66. A vector comprising a nucleic acid molecule encoding any one of SEQ ID NOs: 07, 08, 09, 11, 13, 15, 17, 19, 20, 22, 24, 26, 29, 31, 33, 35 and 37.

67. A nucleic acid molecule selected from any one of SEQ ID NOs: 30, 32, 34, 36 or 38.

68. A vector comprising a nucleic acid molecule selected from SEQ ID NOs: 30, 32, 34, 36 or 38.

69. A mRNA comprising a nucleic acid sequence selected from SEQ ID NOs: 30, 32, 34, 36 or 38.

70. A pharmaceutical composition comprising a nucleic acid molecule, vector or mRNA of any one of claims 63-69.

71. The pharmaceutical composition of claim 70 comprising a lipid nanoparticle.
